(19) United States
(12) Patent Application Publication
(10) Pub. No.: US 2024/0352501 A1
(43) Pub. Date: Oct. 24, 2024

(54) CELLS COMPRISING MOGROSIDE PATHWAY ENZYMES AND USES THEREOF

(71) Applicant: The State of Israel, Ministry of Agriculture & Rural Development, Agricultural Research Organization, Rishon-LeZion (IL)

(72) Inventors: Maxim ITKIN, Kibbutz HaOgen (IL); Rachel DAVIDOVICH-RIKANATI, Alonei Abba (IL); Shahar COHEN, Rishon-LeZion (IL); Vitaly PORTNOY, Nesher (IL); Adi DORON-FAIGENBOIM, Tel Aviv-Yafo (IL); Marina PETREIKOV, Rishon-LeZion (IL); Shmuel SHEN, Moshav Beit-Elazari (IL); Yaakov TADMOR, Timrat (IL); Yosef BURGER, Haifa (IL); Efraim LEWINSOHN, Timrat (IL); Nurit KATZIR, Kiryat-Tivon (IL); Arthur Aaron SCHAFFER, Hashmonaim (IL); Elad OREN, Beit Shearim (IL)

(73) Assignee: The State of Israel, Ministry of Agriculture & Rural Development, Agricultural Research Organization, Rishon-LeZion (IL)

(21) Appl. No.: 18/757,644

(22) Filed: Jun. 28, 2024

Related U.S. Application Data
(62) Division of application No. 17/563,127, filed on Dec. 28, 2021, now Pat. No. 12,071,646.
Application No. 17/563,127 is a division of application No. 15/510,708, filed on Mar. 13, 2017, now abandoned.
(60) Provisional application No. 62/089,929, filed on Dec. 10, 2014.
(60) Provisional application No. 62/048,924, filed on Sep. 11, 2014.

(51) Int. Cl. C12P 33/12; A23L 2/60; A23L 27/30; C07J 17/00; C12N 9/02; C12N 9/14; C12N 9/88; C12N 9/90; C12P 19/56; C12P 33/20

(52) U.S. Cl. CPC C12P 33/12; A23L 2/60; A23L 27/36; C07J 17/005; C12N 9/0071; C12N 9/0083; C12N 9/14; C12N 9/88; C12N 9/90; C12P 19/56; C12P 33/20; C12Y 114/99007; C12Y 204/02017; C12Y 402/01; C12Y 504/99033; A23V 2002/00

(57) ABSTRACT

Isolated mogroside and mogrol biosynthetic pathway enzyme polypeptides useful in mogroside biosynthesis are provided. Mogroside biosynthetic pathway enzymes of the invention include squalene epoxidase (SE), expoxy hydratase (EH), cytochrome p450 (Cyp), cucurbitadienol synthase (CDS) and udp-glucosyl-transferase (UGT), Also provided are methods of producing a mogroside using the isolated mogroside and mogrol biosynthetic enzyme polypeptides, the methods comprising contacting a mogrol and/or a glycosylated mogrol (mogroside) with at least one UDP glucose glucosyl transferase (UGT) enzyme polypeptide of the invention catalyzing glucosylation of the mogrol and/or the glucosylated mogrol to produce a mogroside with an additional glucosyl moietie(s), thereby producing the mogroside. Alternatively or additionally provided is a method of synthesizing a mogrol, the method comprising contacting a mogrol precursor substrate with one or more mogrol biosynthetic pathway enzyme polypeptides as described herein catalyzing mogrol synthesis from the mogrol precursor substrate, thereby synthesizing the mogrol.

### RELATED APPLICATIONS

This application is a divisional of U.S. patent application Ser. No. 17/563,127, filed on Dec. 28, 2021, which is a divisional of U.S. patent application Ser. No. 15/510,708 filed on Mar. 13, 2017, which is a National Phase of PCT Patent Application No. PCT/IL2015/050933 having International Filing Date of Sep. 10, 2015, which claims the benefit of priority under 35 USC § 119 (e) of U.S. Provisional Patent Application Nos. 62/089,929 filed on Dec. 10, 2014 and 62/048,924 filed on Sep. 11, 2014. The contents of the above applications are all incorporated by reference as if fully set forth herein in their entirety.

### SEQUENCE LISTING STATEMENT

The XML file, entitled 100400SequenceListing.xml, created on

Jun. 19, 2024, comprising 71,772 bytes, submitted concurrently with the filing of this application is incorporated herein by reference.

### FIELD AND BACKGROUND OF THE INVENTION

The present invention, in some embodiments thereof, relates to methods of producing mogrosides and compositions comprising same and uses thereof.

Mogrosides are triterpene-derived specialized secondary metabolites found in the fruit of the Cucurbitaceae family plant Siraitia grosvenorii (Luo Han Guo). Their biosynthesis in fruit involves number of consecutive glucosylations of the aglycone mogrol to the final sweet products mogroside V and mogroside VI (FIG. 1).

Mogroside V has been known in the food industry as a natural non-sugar food sweetener, with a sweetening capacity of ˜250 times that of sucrose (Kasai R., et al., Sweet cucurbitane glycosides from fruits of Siraitia siamensis (chi-zi luo-han-guo), a Chinese folk medicine. Agric Biol Chem 1989, 53 (12): 3347-3349.). Moreover, additional health benefits of mogrosides have been revealed in recent studies (Li et al., Chemistry and pharmacology of Siraitia grosvenorii: a review. Chin J Nat Med. 2014 12 (2): 89-102.).

The parent aglycone compound mogrol is derived by successive hydroxylations of cucurbitadienol, the initial product of the stereospecific triterpene synthase, cucurbitadienol synthase. Cucurbitadienol is subsequently hydroxylated, by as yet undetermined enzymes, at the C11, C24 and C25 positions, leading to mogrol (FIG. 1). The trans C24, C25 di-hydroxylations are rare among the triterpenoid cucurbitadienol derivatives (Chen J C, et al., Cucurbitacins and cucurbitane glycosides: structures and biological activities. Nat. Prod. Rep. 2005, 22, 386-399) and thus makes the identification of the enzymes responsible a challenge. The mogrol is subsequently glucosylated at the C3 and C24 positions to varying degrees, from 1 to 6 glucosyl groups, in a temporally successive pattern during fruit development and the glucosylated mogrol compounds are termed mogrosides. The sweetness strength of the mogrosides increases with the additional glucose moieties such that M6 (with 6 glucosyl groups) is sweeter than M5, followed by M4, respectively (Kasai R., et al., Sweet cucurbitane glycosides from fruits of Siraitha siamensis (chi-zi luo-han-guo), a Chinese folk medicine. Agric Biol Chem 1989, 53 (12): 3347-3349). The purified mogroside V, has been approved as a high-intensity sweetening agent in Japan (Jakinovich, W., Jr., Moon, C., Choi, Y. H., & Kinghorn, A. D. 1990. Evaluation of plant extracts for sweetness using the Mongolian gerbil. Journal of Natural Products, 53, 190-195) and the extract has gained generally recognized as safe (GRAS) status in the USA as a non-nutritive sweetener and flavor enhancer.

Extraction of mogrosides from the fruit can yield a product of varying degrees of purity, often accompanied by undesirable aftertaste. In addition, yields of mogroside from cultivated fruit are limited due to low plant yields and particular cultivation requirements of the plant. It is therefore advantageous to be able to produce sweet mogroside compounds via biotechnological processes.

Additional background art includes:

WO2013/076577 discloses enzymes of the UGT family (UDPglucose glycosyl transferase) from Arabidopsis thaliana and Stevia rebaudiana, plants which do not naturally produce mogroside. Four of these enzymes were capable of performing glycosylation of the aglycone mogrol, specifically the addition of single glucose moieties at the C24 positions to produce M1b. The fifth enzyme UGT73C5 from Stevia rebaudiana showed glycosylation at both C3 and C24.

WO 2014086842 discloses the cucurbitadienol synthase, the cyp450 that catalyzes C-11 OH production and some UGT polypeptides from Siraitia grosvenorii, shows that these enzymes function in yeast, and provide as well for methods for producing mogrosides. In addition, they also disclose 2 epoxide hydrolases, and demonstrate their ability to hydrate epoxysqualene, suggesting that they can hydrate epoxy cucurbitadienol as well. In particular the invention proposes various biosynthetic pathways useful for mogroside production and enzymes useful for mogroside production are provided. Furthermore, the invention provides recombinant hosts useful in performing the methods of the invention. Tang et al., An efficient approach to finding Siraitia grosvenorii triterpene biosynthetic genes by RNA-seq and digital gene expression analysis. BMC Genomics. 2011; 12:343.

### SUMMARY OF THE INVENTION

According to an aspect of some embodiments of the present invention there is provided an isolated uridine diphospho-glucosyl transferase enzyme (UGT) polypeptide comprising an amino acid sequence, wherein the polypeptide catalyzes primary glucosylation of mogrol at C24 and primary glucosylation of mogroside at C3.

According to some embodiments of the present invention the isolated UGT polypeptide catalyzes:
(a) primary glucosylation of mogrol at C24;
(b) primary glucosylation of mogroside at C3; and
(c) branching glucosylation of mogroside at C3.

According to some embodiments of the present invention the amino acid sequence at least 34% identical to SEQ ID NO: 34.

According to some embodiments of the present invention the amino acid sequence is as set forth in SEQ ID NO: 34.

According to an aspect of some embodiments of the present invention there is provided an isolated uridine diphospho-glucosyl transferase enzyme (UGT) polypeptide comprising an amino acid sequence, wherein the polypeptide catalyzes branching glucosylation of mogroside at the (1-2) and (1-6) positions of C3 and branching glucosylation of mogroside at the (1-2) and (1-6) positions of C24.

According to an aspect of some embodiments of the present invention there is provided an isolated uridine diphospho-glucosyl transferase enzyme (UGT) polypeptide comprising an amino acid sequence wherein the polypeptide catalyzes branching glucosylation of mogroside M5 to mogroside M6.

According to some embodiments of the present invention the isolated UGT polypeptide catalyzes:
(a) branching glucosylation of mogroside at the (1-2) and (1-6) positions of

C3;
(b) branching glucosylation of mogroside at the (1-2) and (1-6) positions of C24, and
(c) branching glucosylation of mogroside M5 to mogroside M6.

According to some embodiments of the present invention the amino acid sequence is at least 89% identical to SEQ ID NO: 38.

According to an aspect of some embodiments of the present invention the amino acid sequence is as set forth in SEQ ID NO: 38.

According to an aspect of some embodiments of the present invention there is provided an isolated uridine diphospho-glucosyl transferase enzyme (UGT) polypeptide comprising an amino acid sequence, wherein the polypeptide catalyzes branching glucosylation of mogroside IV (M4) to mogroside V (M5).

According to some embodiments of the present invention the amino acid sequence is selected from the group consisting of a sequence at least 34% identical to SEQ ID NO: 34, a sequence at least 84% identical to SEQ ID NO: 6 and a sequence at least 89% identical to SEQ ID NO:38.

According to some embodiments of the present invention the amino acid sequence is as set forth in SEQ ID NO:6.

According to some embodiments of the present invention the amino acid sequence is as set forth in SEQ ID NO:38.

According to some embodiments of the present invention the amino acid sequence is as set forth in SEQ ID NO: 34.

According to some embodiments of the present invention the UGT is a plant UGT.

According to some embodiments of the present invention the plant is a plant of the Cucurbitaceae family.

According to some embodiments of the present invention the plant is Siraitia grosvenorii.

According to an aspect of some embodiments of the present invention there is provided an isolated squalene epoxidase (SQE) polypeptide comprising an amino acid sequence at least 94% identical to SEQ ID NO: 14 or 89% identical to SEQ ID NO: 16, wherein the polypeptide catalyzes diepoxysqualene synthesis from squalene or oxidosqualene.

According to some embodiments of the present invention the amino acid sequence is as set forth in SEQ ID NO: 14 or SEQ ID NO: 16.

According to some embodiments of the present invention the SQE is a plant SQE.

According to an aspect of some embodiments of the present invention there is provided an isolated epoxide hydrolase (EH) polypeptide comprising an amino acid sequence at least 75% identical to SEQ ID NO: 18, SEQ ID NO: 22 or SEQ ID NO: 24, wherein the polypeptide catalyzes 3, 24, 25 trihydroxy cucurbitadienol synthesis from 3-hydroxy, 24-25 epoxy cucurbitadienol.

According to some embodiments of the present invention the amino acid sequence is as set forth in any one of SEQ ID NO: 18, SEQ ID NO: 22 and SEQ ID NO: 24.

According to some embodiments of the present invention the EH is a plant EH.

According to an aspect of some embodiments of the present invention there is provided a method of synthesizing a mogrol or mogrol precursor product from a mogrol precursor substrate, the method comprising contacting at least one mogrol precursor substrate with a mogroside pathway enzyme, wherein:
(a) when the mogrol precursor product comprises diepoxy squalene and the enzyme comprises a squalene epoxidase polypeptide as described in any one of claims 18-20, thereby producing diepoxy squalene,
(b) when the mogrol precursor product comprises 3 hydroxy, 24-25 epoxy cucurbitadienol and the mogrol precursor substrate comprises diepoxy squalene, the mogrol pathway enzyme comprises a cucurbitadienol synthetase polypeptide as set forth in SEQ ID NO: 12 or 60% homologous or identical thereto, thereby producing a 3 hydroxy, 24-25 epoxy cucurbitadienol,
(c) when the product comprises 3, 24, 25 trihydroxy cucurbitadienol and the substrate comprises 3-hydroxy, 24-25 epoxy cucurbitadienol, the mogrol pathway enzyme comprises an epoxy hydratase polypeptide as described in any one of claims 21-23, thereby producing a 3, 24, 25 trihydroxy cucurbitadienol,
(d) when the product comprises mogrol and the mogrol precursor substrate comprises 3, 24, 25 trihydroxy cucurbitadienol, the mogrol pathway enzyme is Cytochrome P 450 enzyme as set forth in SEQ ID NO: 10 or 60% homologous or identical thereto, thereby producing 3, 11, 24, 25 tetrahydroxy cucurbitadienol (mogrol).

According to some embodiments of the present invention the Cytochrome P 450 enzyme comprises an amino acid sequence as set forth in SEQ ID NO: 10.

According to some embodiments of the present invention producing the mogrol product comprises at least one of:
(i) contacting the squalene or oxido squalene with the squalene epoxidase enzyme polypeptide, thereby producing diepoxy squalene;
(ii) contacting the diepoxy squalene with a cucurbitadienol synthase, thereby producing 3 hydroxy, 24-25 epoxy cucurbitadienol;
(iii) contacting the 3 hydroxy, 24-25 epoxy cucurbitadienol with the epoxy hydratase enzyme, thereby producing 3, 24, 25 trihydroxy cucurbitadienol; and
(iv) contacting the 3, 24-25 trihydroxy cucurbitadienol with the Cytochrome P 450 enzyme, thereby producing the mogrol product (3, 11, 24, 25 tetrahydroxy cucurbitadienol).

According to some embodiments of the present invention producing the mogrol product comprises at least (i) and (iv), at least (ii) and (iv), at least (iii) and (iv), at least (i), (ii) and (iii), at least (i), (ii) and (iv), at least (i), (iii) and (iv), at least (ii), (iii) and (iv).

According to some embodiments of the present invention producing the mogrol product comprises all of (i) (ii), (iii) and (iv).

According to an aspect of some embodiments of the present invention there is provided a method of synthesizing a mogroside, the method comprising contacting at least one UGT polypeptide of the invention or a combination thereof with at least one UGT substrate mogroside precursor.

According to some embodiments of the present invention the at least one UGT polypeptide comprises the UGT polypeptide polypeptide catalyzing primary glucosylation of mogrol at C24 and primary glucosylation of mogroside at C3 of the invention.

According to some embodiments of the present invention the at least one UGT polypeptide comprises the UGT polypeptide having an amino acid sequence as set forth in SEQ ID NO: 34.

According to some embodiments of the present invention the at least one UGT polypeptide comprises the UGT polypeptide of the invention catalyzing branching glucosylation of mogroside at the (1-2) and (1-6) positions of C3 and branching glucosylation of mogroside at the (1-2) and (1-6) positions of C24, and/or catalyzing branching glucosylation of mogroside M5 to mogroside M6.

According to some embodiments of the present invention the at least one UGT polypeptide comprises a UGT polypeptide of having an amino acid sequence as set forth in SEQ ID NO: 38.

According to some embodiments of the present invention the at least one UGT polypeptide comprises the UGT polypeptide of the invention catalyzing branching glucosylation of mogroside IV (M4) to mogroside V (M5).

According to some embodiments of the present invention the at least one UGT polypeptide comprises the UGT polypeptide having an amino acid sequence selected from the group consisting of a sequence at least 34% identical to SEQ ID NO: 34, a sequence at least 84% identical to SEQ ID NO: 6 and a sequence at least 89% identical to SEQ ID NO:38.

According to some embodiments of the present invention the at least one UGT polypeptide comprises the UGT polypeptide having an amino acid sequence as set forth in SEQ ID NO: 34 and the UGT polypeptide having an amino acid sequence as set forth in SEQ ID NO: 38.

According to some embodiments of the present invention, wherein the UGT substrate mogroside precursor substrate is a mogrol, the method comprises:
(a) producing a mogrol according to the method of the invention, and
(b) synthesizing the mogroside from the mogrol according to the method of synthesizing mogroside of the invention.

According to some embodiments of the present invention the mogroside is selected from the group consisting of mogroside I-A1, mogroside I-E1, mogroside IIE, mogroside III, siamenoside, mogroside V and mogroside VI.

According to some embodiments of the present invention, the method, further comprises isolating the mogroside.

According to some embodiments of the present invention the method is performed in a recombinant cell exogenously expressing at least one of the mogoside pathway enzyme polypeptides of the invention or any combination thereof.

According to some embodiments of the present invention the at least one polypeptide is selected from the group consisting of a polypeptide having an amino acid sequence as set forth in SEQ ID NO: 34, a polypeptide having an amino acid sequence as set forth in SEQ ID NO: 38, a polypeptide having an amino acid sequence as set forth in SEQ ID NO: 14 or 16 and a polypeptide having an amino acid sequence as set forth in SEQ ID NO: 18, 22 or 24.

According to an aspect of some embodiments of the present invention there is provided a composition comprising a mogroside generated according to the method of mogroside biosynthesis of the invention.

According to an aspect of some embodiments of the present invention there is provided an isolated polynucleotide comprising a nucleic acid sequence encoding the isolated polypeptide of any one of the SE, CDS, EH, Cyt p450 and UGT enzyme polypeptides of the invention.

According to some embodiments of the present invention nucleic acid sequence is selected from the group consisting of SEQ ID NOs. 5, 9, 11, 13, 15, 17, 21, 23, 33 and 37.

According to an aspect of some embodiments of the present invention there is provided a nucleic acid construct comprising the isolated polynucleotide of the invention and a cis-acting regulatory element for directing expression of the isolated polynucleotide.

According to some embodiments of the present invention the cis-acting regulatory element comprises a promoter.

According to an aspect of some embodiments of the present invention there is provided a host cell heterologously expressing the isolated polynucleotide of the invention.

According to some embodiments of the present invention the host cell is of a microorganism.

According to some embodiments of the present invention the microorganism is selected from the group of yeast and bacteria.

According to some embodiments of the present invention the host cell is a plant host cell.

According to some embodiments of the present invention the host cell forms a part of a plant.

According to some embodiments of the present invention the plant is a transgenic plant.

According to some embodiments of the present invention the plant is of the Cucurbitacaea family.

According to some embodiments of the present invention the host cell forms a part of a fruit or root of the plant.

According to some embodiments of the present invention the host cell produces a mogroside or mogroside precursor in the host cell.

According to an aspect of some embodiments of the present invention there is provided a cell lysate of the host cell of the invention.

According to an aspect of some embodiments of the present invention there is provided a composition enriched in mogroside VI to a total concentration of mogroside VI of at least 10% (wt/wt).

According to an aspect of some embodiments of the present invention there is provided a composition comprising mogroside VI (M6) and mogroside II (M2).

According to an aspect of some embodiments of the present invention there is provided a composition comprising mogroside V (M5), VI (M6) and mogroside II (M2)

According to some embodiments of the present invention concentration of the mogroside VI or mogroside V is sufficient to cause an enhancement in flavor.

According to some embodiments of the present invention a concentration of the mogroside VI is at least 0.2 ppm.

According to some embodiments of the present invention the composition is a sweetener.

According to some embodiments of the present invention the composition further comprises a flavor ingredient selected from the group consisting of sucrose, fructose, glucose, high fructose corn syrup, xylose, arabinose, rhamnose, erythritol, xylitol, mannitol, sorbitol, inositol, AceK, aspartame, neotame, sucralose, saccharine, naringin dihydrochalcone (NarDHC), neohesperidin dihydrochalcone (NDHC), rubusoside, rebaudioside A, stevioside, stevia, trilobtain.

According to some embodiments of the present invention the composition is a consumable composition.

According to some embodiments of the present invention the composition further comprises one or more additional flavor ingredients.

According to some embodiments of the present invention the composition is a beverage.

According to some embodiments of the present invention the beverage is selected from the group consisting of an aqueous beverage, enhanced/slightly sweetened water drink, mineral water, carbonated beverage, non-carbonated beverage, carbonated water, still water, soft drink, non-alcoholic drink, alcoholic drink, beer, wine, liquor, fruit drink, juice, fruit juice, vegetable juice, broth drink, coffee, tea, black tea, green tea, oolong tea, herbal tea, cacao, tea-based drink, coffee-based drinks, cacao-based drink, syrup, dairy products, frozen fruit, frozen fruit juice, water-based ice, fruit ice, sorbet, dressing, salad dressing, sauce, soup, and beverage botanical materials, or instant powder for reconstitution.

According to some embodiments of the present invention the composition is Coca-Cola® and the like.

According to some embodiments of the present invention the composition is a solid consumable.

According to some embodiments of the present invention the solid consumable is selected from the group consisting of cereals, baked food products, biscuits, bread, breakfast cereal, cereal bar, dairy product, energy bars/nutritional bars, granola, cakes, cookies, crackers, donuts, muffins, pastries, confectioneries, chewing gum, chocolate, fondant, hard candy, marshmallow, pressed tablets, snack foods, botanical materials (whole or ground), and instant powders for reconstitution.

According to some embodiments of the present invention the composition is a foodstuff.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an illustration (adapted from Tang et al., An efficient approach to finding Siraitia grosvenorii triterpene biosynthetic genes by RNA-seq and digital gene expression analysis. BMC Genomics. 2011; 12:343). Putative mogrosides biosynthesis pathway in Siraitia grosvenorii. AACT: acetyl-CoA acetyltransferase, EC:2.3.1.9; HMGS: hydroxymethylglutaryl-CoA synthase, EC:2.3.3.10; HMGR: 3-hydroxy-3-methylglutaryl-coenzyme A reductase, EC:1.1.1.34; MK: mevalonate kinase, EC:2.7.1.36; PMK: phosphomevalonate kinase, EC:2.7.4.2; MVD: diphosphomevalonate decarboxylase, EC:4.1.1.33; DXS: 1-deoxy-D-xylulose-5-phosphate synthase, EC:2.2.1.7; DXR: 1-deoxy-D-xylulose-5-phosphate reductoisomerase, EC:1.1.1.267; MCT: 2-C-methyl-D-erythritol 4-phosphate cytidylyltransferase, EC:2.7.7.60; CMK: 4-diphosphocytidyl-2-C-methyl-D-erythritol kinase, EC:2.7.1.148; MCS: 2-C-methyl-D-erythritol 2,4-cyclodiphosphate synthase, EC:4.6.1.12; HDS: 4-hydroxy-3-methylbut-2-enyl diphosphate synthase, EC:1.17.7.1; IDS: 4-hydroxy-3-methylbut-2-enyl diphosphate reductase (isopentenyl/dimethylallyl diphosphate synthase), EC:1.17.1.2; IPI: isopentenyl-diphosphate delta-isomerase, EC:5.3.3.2; GPS: geranyl diphosphate synthase, EC:2.5.1.1; FPS: farnesyl diphosphate synthase/farnesyl pyrophosphate synthetase, EC:2.5.1.10; SQS: squalene synthetase; CAS: cycloartenol synthase, EC:2.5.1.21; SQE: squalene epoxidase, EC:1.14.99.7; CS: cucurbitadienol synthase, EC:5.4.99.8; P450: cytochrome P450, EC:1.14.-.-; and UDPG: UDP-glucosyltransferase, EC:2.4.1. E.C. 2.4.1 are UGTs;

FIG. 2 is an illustration of the proposed pathway of mogroside synthesis in Siraitia fruit;

FIG. 3 illustrates the numbering system for compounds related to 2,3;22,23-dioxidosqualene (linear, above) and mogrol (cyclized, below), showing the key numbered carbons (blue);

FIGS. 4A-4B are graphic illustrations showing mogroside levels in a course of Siraitia fruit development and ripening. Note the progressive loss of M2 and M3, and concomitant increase in M4 and M5 (FIG. 4B), indicating sequential glucosylation. Values are expressed as relative to highest mogroside content in FIG. 4A, and the relative amount of each compound in FIG. 4B, based on peak area of the chromatograms;

FIGS. 5A and 5B are graphs illustrating the relative expression patterns of squalene epoxidase 1 (FIG. 5A) and squalene epoxidase 2 (FIG. 5B). In the developing Siraitia fruit showing relatively high expression in the youngest fruit;

FIGS. 6A-6C show HPLC-MS chromatograms illustrating production of both 2,3-monooxidosqualene and 2,3;22,23-dioxidosqualene in the yeast host (FIG. 6A); cyclicization of these substrates to both cucurbitadienol and 24,25-epoxycucurbitadienol in yeast hosts expressing Siraitia cucurbitadienol synthase (SgCDS) (FIG. 6B). FIG. 6C-substrate and product standards. Both cucurbitadienol and 24,25-epoxycucurbitadienol were identified by MS and NMR in the yeast extracts;

FIG. 7 is a hierarchical cluster heat map of expression patterns of the 8 epoxide hydrolase genes expressed in the developing Siraitia fruit. The five stages of fruit development presented are 15, 34, 51, 77 and 103 days and correspond to the fruit development stages in FIGS. 4A and 4B;

FIGS. 8A-8B illustrate the effect of epoxide hydrolase expression on 24,25-dihydroxycucurbitadienol. FIG. 8A shows LC-MS chromatograms demonstrating the increase in 24,25-dihydroxycucurbitadienol due to the expression of epoxide hydrolase genes in extracts of yeast expressing cucurbitadienol synthase (SgCDS). The top three chromatograms show the effect of EPH1, 2 and 3 (SEQ ID NOs. 17, 19 and 21), respectively. The bottom chromatogram shows the control yeast harboring the CDS without the additional EPH genes. FIG. 8B is a graph showing the relative levels of 24,25-dihydroxycucurbitadienol (compound 1 of FIG. 8A) and 24,25-epoxycucurbitadienol (compound 3 of FIG. 8A) in the control and EPH-expressing yeast lines;

FIG. 9 is an identity-similarity matrix of reported Siraitia Epoxide Hydrolase protein sequences. The sequences in green [encoded by contig_6184 (SEQ ID NO: 39) and contig_8262 (SEQ ID NO: 40)] are from the database reported in Tang et al., (2011) and reported as SEQ ID NOs. 38 and 40, respectively of US2015/0064743. Sequences encoded by contigs 101438, 102175, 102581 and 22474 are SEQ ID NOs. 41, 42, 43 and 44, respectively. The matrix was prepared using the ClustalOmega program (www(dot)ebi(dot)ac(dot)uk/Tools/msa/clustalo/);

FIG. 10 is a hierarchical cluster heat map of expression patterns of the cytochrome P450 genes expressed in the developing Siraitia fruit. The five stages of fruit development presented are 15, 34, 51, 77 and 103 days and correspond to the fruit development stages in FIGS. 4A and 4B; Approximately 40 candidates were functionally expressed and assayed for cucurbitadienol hydroxylation activity;

FIGS. 11A-11C are HPLC-MS chromatograms showing the C11-hydroxylation of cucurbitadienol by the Cytochrome P 450 cyp102801 (SEQ ID NO: 10) (FIG. 11A). FIG. 11B shows a chromatogram of the extract from the yeast line (devoid of CDS (cucurbitadienol synthase expression) expressing cyp102801. FIG. 11C shows a chromatogram of yeast extract from yeast hosts expressing CDS but not cyp102801;

FIG. 12 is a list of the mogroside substrates used for the screening of glucosyltransferase activity, identifying the substrates according to various nomenclature, and their source and the method used to identify them;

FIGS. 13A-13B show a phylogenetic analysis of Uridine diphosphate glucosyl transferase (UGT) sequences of some embodiments of the invention. FIG. 13A is a phylogenetic analysis of UGT protein sequences from a Clustal Omega alignment. FIG. 13B is a phylogenetic tree of Siraitia UGTs. Branches, corresponding to same gene family are marked by color. Siraitia UGTs that were shown to glucosylate mogrol and mogrosides in this application are boxed in red;

FIG. 14 is a hierarchical cluster heat map of expression patterns of the UGT genes expressed in the developing Siraitia fruit. The five stages of fruit development presented are 15, 34, 51, 77 and 103 days and correspond to the fruit development stages in FIGS. 4A and 4B. Approximately 100 candidates were functionally expressed and assayed for UGT activity with the mogroside substrates;

FIG. 15A-15C are a schematic of UGT enzyme-sugar-acceptor molecule activities, based on products identified from cell-free glucosylation reactions with individual recombinant UGT enzymes expressed in E. coli and mogrol and mogroside substrates. FIG. 15A shows primary glucosylations, while FIG. 15B shows branching glucosylation and FIG. 15C shows the primary glucosylations that the branching enzymes presented in FIG. 15B perform. Schematic representation of sugar molecules are shown as circles, when each pair of cyclic cucrbitane rings are represented by blue ovals (rings A and B are schematically combined into the lower oval and rings C and D are combined into the upper oval), and the non-cyclic branched portion of the cucurbitadienol molecule leading towards C-24 and C-25 is represented by a short line. Newly attached glucose moieties from the UGT reaction are marked by green circles, glucose molecules derived from the substrate are in red, and a purple circle indicates where the position of the glucose added was identified by NMR as position C-25 glucose. When the circle points up (diagonally left or right) it represents a (1-6) glycosidic bond, whereas down-pointing circle (diagonally left or right) represents a (1-2) glycosidic bond. Circle pointing left represents a (1-4) glycosidic bond. Asterisk indicates trace amounts of substance;

FIG. 16 shows HPLC/DAD chromatograms of the mogroside products synthesized from each of the primary glucosylation enzymes upon inclusion of the aglycone mogrol (M) in the cell-free reaction media as described in FIGS. 15A-15C. The top three enzymes each synthesize the C-3 glucosidic mogrol, M1E1. UGT85E5 (269-1) synthesizes both the C-24 glucosidic mogrol, MIA and the C3, C24-diglucoside, M2E. The products were identified by MS and by NMR;

FIGS. 17A-17D show HPLC/DAD chromatograms showing that UGT94C9 (289-3) catalyzes cell-free production of Mogroside VI using Mogroside V as a substrate [Peak eluting at 1.9 min (m/z=1449.7113)]. FIG. 17A illustrates the accumulation of Mogroside VI in the reaction mixture, compared to inactive enzyme control (FIG. 17B). Residual Mogroside V that was not completely converted to Mogroside VI in reaction mix, elutes at 2.1 min. (FIG. 17A). FIG. 17C is a chromatogram of a standard of Mogroside VI (identified as M6-II). The reaction products were verified using LC-MS. The resulting spectrum is shown for two Mogroside VI (M6) compounds, Mogroside V (M5) from 17A and Mogroside VI (M6) standard. To discriminate between two Mogrosides VI they were marked M6-I (eluting at 1.5 min) and M6-II (eluting at 1.9 min);

FIG. 18 is a similarity and identity pairwise matrix of alignments of UGT amino acid sequences. The matrix was calculated using MatGAT 2.02 (www(dot)bitincka(dot)com/ledion/matgat/) run with BLOSUM62. Percentage similarity between the amino acid sequences is presented to the left and below the “100% self” diagonal, and percent identity presented to the right and above the “100% self” diagonal;

FIGS. 19A and 19B are chromatograms showing that UGT94-289-3 performs sequential glucosylations to generate Siamenoside and Mogroside 4A from Mogroside 2E in a cell free reaction system. FIG. 19A is an example of a LC-MS chromatogram of the products from the reaction with Mogroside 1A as substrate in the presence of UGT74-345-2 and UGT94-289-3. FIG. 19B shows the spectra for Mogroside 3x and for two Mogroside IV moieties: Mogroside IVA and Siamenoside;

FIG. 20 shows the expression pattern of a candidate squalene epoxidase homologue from S. grosvenorii, encoded by contig 19984, which was not selected due to the late expression in fruit development, as well as its sharp decline thereafter;

FIG. 21 shows the expression pattern of a candidate epoxy hydratase homologue from S. grosvenorii, encoded by contig 73966 (SEQ ID NO:17), selected for high and early expression in fruit development, and the gradual decline in expression during ripening;

FIG. 22 shows the expression pattern of a candidate epoxy hydratase homologue from S. grosvenorii, encoded by contig 86123 (SEQ ID NO: 19), selected for high and early expression in fruit development and gradual decline in expression during ripening;

FIG. 23 shows the expression pattern of a candidate epoxy hydratase homologue from S. grosvenorii, encoded by contig 102640 (SEQ ID NO: 3), selected for high and early expression in fruit development and gradual decline in expression during ripening;

FIG. 24 shows the expression pattern of a candidate epoxy hydratase homologue from S. grosvenorii, encoded by contig 28382 (SEQ ID NO: 4), selected for high and early expression in fruit development and gradual decline in expression during ripening.

### DESCRIPTION OF SPECIFIC EMBODIMENTS OF THE INVENTION

The present invention, in some embodiments thereof, relates to methods of producing mogrol, mogrosides and compositions comprising same and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

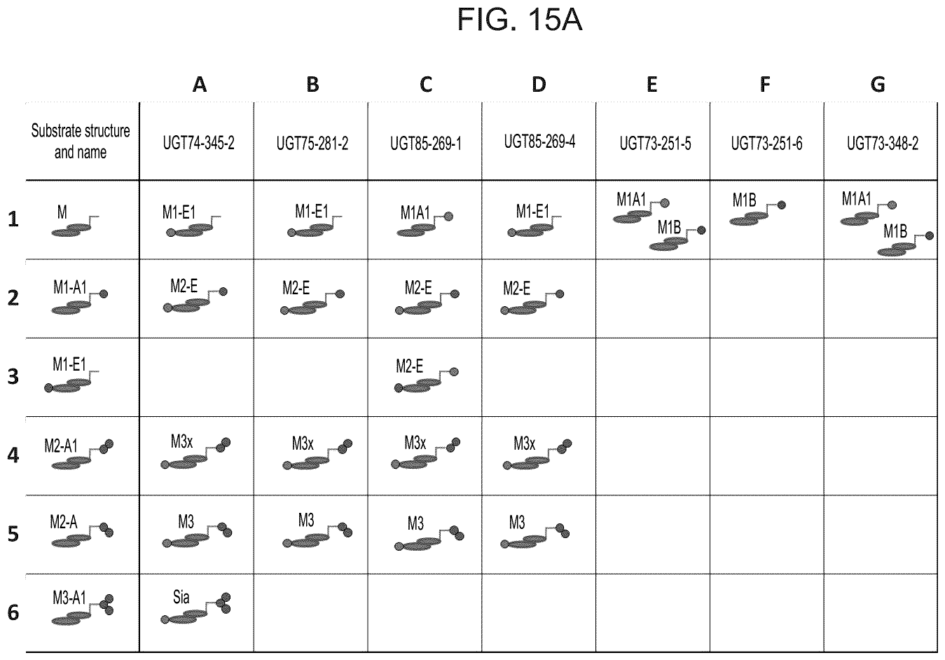

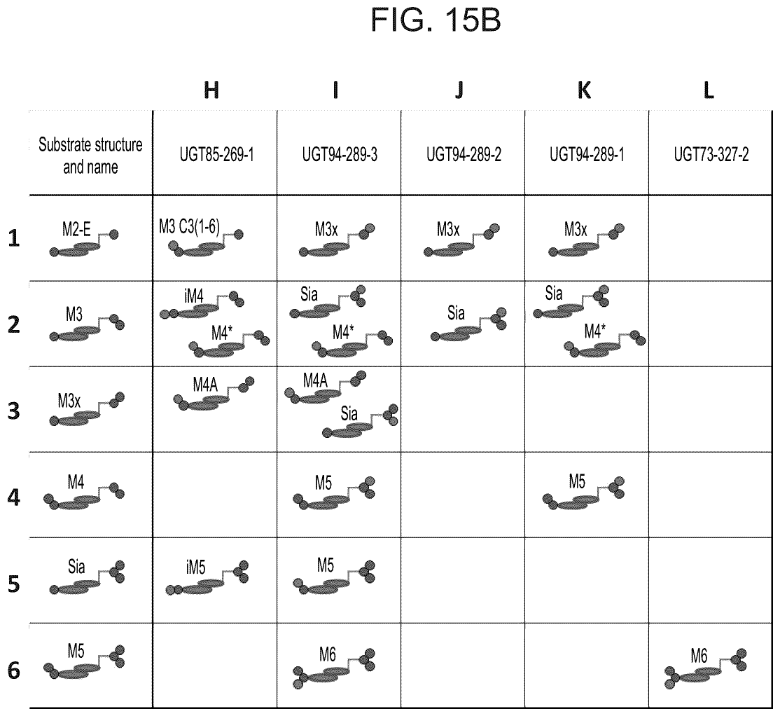

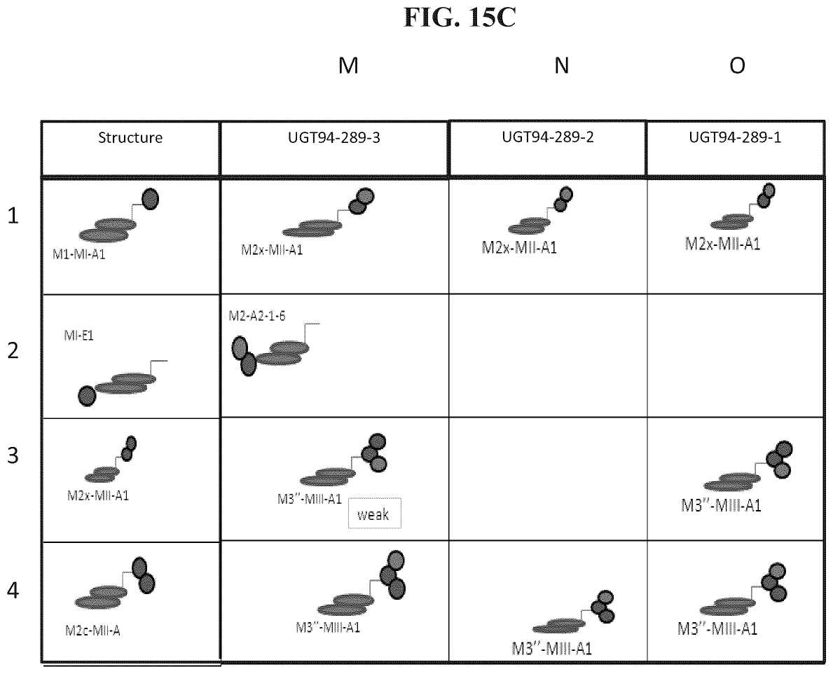

Mogrol (3, 11, 24, 25 tetrahydroxy cucurbitadienol) is the substrate for the biosynthesis of mogrosides (glycosylated mogrol), the glycosylation of carbons at positions 3, 24 and/or 25 being catalyzed by glucosyltransferase enzymes, such as uridine-5-dipospho-dependent glucosyltransferase (UGT). Mogrol biosynthesis requires the steroid precursor squalene as a substrate, and involves cyclization and hydroxylation of residues. The exact biochemical pathways are not currently known, however, the instant inventors have identified a mogrol synthetic pathway likely prominent in the endogenous biosynthesis of mogrol, have identified S. grosvenorii enzymes critical to the production of mogrol, mogrol precursors, mogroside precursors and mogrosides, have successfully reconstituted significant portions of the biosynthetic pathway with the recombinantly synthesized mogrol/mogroside pathway enzymes (see Examples 5 and 6, and FIGS. 15A-15C). Based on the combined metabolic profiling, functional expression and protein modeling results the present inventors suggest the following metabolic pathway for S. grosvenorii mogroside biosynthesis: During the initial stage of fruit development squalene is metabolized to the diglucosylated M2, via the progressive actions of squalene synthase, squalene epoxidase, cucurbitadienol synthase, epoxide hydrolase, cytochrome p450 (cyp102801) and UGT85. During fruit maturation there is the progressive activity of the UGT94 members, and perhaps also the UGT85, adding branched glucosyl groups to the primary glucosyl moieties of M2, leading to the sweet-flavored M4, M5 and M6 compounds.

Mogroside synthesis from mogrol is initiated by primary glucosylation of the mogrol molecule at carbons C3 and C24, and proceeds with further additions of glucose moieties, all catalyzed by uridine diphospho-glucosyl transferases (EC 2.4.1). The present inventors have unexpectedly uncovered key UTG enzymes having catalytic activity which may be critical to the S. grosvenorii mogroside biosynthesis.

Thus, according to some embodiments of some aspects of the invention there is provided an isolated uridine diphospho-glucosyl transferase enzyme (UGT) polypeptide comprising an amino acid sequence, wherein the polypeptide catalyzes primary glucosylation of mogrol at C24 and primary glucosylation of mogroside at C3. The present inventors have shown that this UGT is promiscuous in its substrate specificity: thus, in some embodiments, using mogrol as a substrate, the isolated UGT polypeptide can catalyze primary glycosylation of mogrol at C24, can catalyze primary glucosylation of a C24 glucosylated mogroside at C3, and can catalyze branched glucosylation of a mogroside. In a specific embodiment, the branching glucosylation is on a primary glucose of C3.

The present inventors have identified this UGT polypeptide as a member of the UGT85 family. In some embodiments, the isolated UGT polypeptide catalyzing primary glucosylation of mogrol at C24 and primary glucosylation of mogroside at C3 comprises an amino acid sequence at least 34% identical to SEQ ID NO: 34. In some embodiments, the amino acid sequence is at least 34% homologous to SEQ ID NO: 34. In some embodiments, the isolated UGT polypeptide catalyzing primary glucosylation of mogrol at C24 and primary glucosylation of mogroside at C3 comprises an amino acid sequence having at least 35%, at least 37%, at least 40%, at least 42%, at least 45%, at least 47%, at least 50%, at least 55%, at least 58%, at least 60%, at least 65%, at least 70%, at least 75%, at least 78%, at least 80%, at least 83%, at least 88%, at least 90%, at least 91%, at least 92%, at least 93%, at least 94%, at least 95%, at least 96%, at least 97%, at least 98%, at least 99%, or 100% homology or identity to SEQ ID NO: 34. In some embodiments, the UTG polypeptide comprises an amino acid sequence having homology or identity in the range of 34-100%, 40-90%, 37-85%, 45-80%, 50-75%, 55-65%, 80-90%, 93-100% to SEQ ID NO: 34. In a specific embodiment, the amino acid sequence of the isolated UGT polypeptide catalyzing primary glucosylation of mogrol at C24 and primary glucosylation of mogroside at C3 is as set forth in SEQ ID NO:34. In some cases, SEQ ID NO:34 is also referred to as UGT85E5, 85E5, and UGT85-269-1.

The present inventors have identified UGT enzymes having branching glucosylation activity critical to mogroside synthesis. Thus, according to some aspects of the invention there is provided an isolated uridine diphospho-glucosyl transferase enzyme (UGT) polypeptide comprising an amino acid sequence wherein the polypeptide catalyzes branching glucosylation of mogroside at the (1-2) and (1-6) positions of C3 and branching glucosylation of mogroside at the (1-2) and (1-6) positions of C24.

According to some aspects of the invention there is provided an isolated uridine diphospho-glucosyl transferase enzyme (UGT) polypeptide comprising an amino acid sequence wherein the polypeptide catalyzes branching glucosylation of mogroside M5 to mogroside M6. This catalytic activity is highly important, since the M6 mogroside is the mogroside with the sweetest taste of all the Siraitia grosvenorii mogroside compounds.

The present inventors have uncovered UGT polypeptides catalyzing branching glucosylation of mogroside at the (1-2) and (1-6) positions of C3 and branching glucosylation of mogroside at the (1-2) and (1-6) positions of C24, as well as branching glucosylation of mogroside M5 to mogroside M6.

The present inventors have identified UGT polypeptides catalyzing branching glucosylation of mogroside at the (1-2) and (1-6) positions of C3 and branching glucosylation of mogroside at the (1-2) and (1-6) positions of C24, and/or branching glucosylation of mogroside M5 to mogroside M6 as members of the UGT94 family. In some embodiments, the isolated UGT polypeptide catalyzing branching glucosylation of mogroside at the (1-2) and (1-6) positions of C3 and branching glucosylation of mogroside at the (1-2) and (1-6) positions of C24, and/or branching glucosylation of mogroside M5 to mogroside M6 comprises an amino acid sequence at least 89% identical to SEQ ID NO: 38. In some embodiments, the amino acid sequence is at least 89% homologous to SEQ ID NO: 38. In some embodiments, the isolated UGT polypeptide catalyzing branching glucosylation of mogroside at the (1-2) and (1-6) positions of C3 and branching glucosylation of mogroside at the (1-2) and (1-6) positions of C24, and/or branching glucosylation of mogroside M5 to mogroside M6 comprises an amino acid sequence having at least 89%, at least 90%, at least 91%, at least 92%, at least 93%, at least 94%, at least 95%, at least 96%, at least 97%, at least 98%, at least 99 or 100% homology or identity to SEQ ID NO: 38. In some embodiments, the UTG polypeptide comprises an amino acid sequence having a homology or identity in the range of 89-100%, 90-100%, 92-85%, 94-80%, 95-100%, 96-100%, 97-100% or 99-100% to SEQ ID NO: 38. In a specific embodiment, the isolated UGT polypeptide catalyzing branching glucosylation of mogroside at the (1-2) and (1-6) positions of C3 and branching glucosylation of mogroside at the (1-2) and (1-6) positions of C24, and/or branching glucosylation of mogroside M5 to mogroside M6 comprises an amino acid sequence as set forth in SEQ ID NO:38. In some cases, SEQ ID NO: 38 is also referred to as UGT94C9 and UGT94-289-3.

Additional UTG enzyme polypeptides which may catalyze branching glucosylation of mogroside M5 to mogroside M6 include, but are not limited to UGT polypeptides comprising an amino acid sequence at least 41% identical or homologous to SEQ ID NO: 8. In some embodiments, the UGT polypeptide comprises an amino acid sequence as set forth in SEQ ID NO: 8. SEQ ID NO: 8 is also referred to as UGT73-327-2, UGT73E7 and EO7.

According to some aspects of the invention there is provided an isolated uridine diphospho-glucosyl transferase enzyme (UGT) polypeptide comprising an amino acid sequence wherein the polypeptide catalyzes branching glucosylation of mogroside IV (M4) to mogroside V (M5). In some embodiments, the isolated UGT polypeptide catalyzing branching glucosylation of mogroside IV (M4) to mogroside V (M5) comprises an amino acid sequence having at least 89%, at least 90%, at least 91%, at least 92%, at least 93%, at least 94%, at least 95%, at least 96%, at least 97%, at least 98%, at least 99 or 100% homology or identity to SEQ ID NO: 38, or an amino acid sequence at least 35%, at least 37%, at least 40%, at least 42%, at least 45%, at least 47%, at least 50%, at least 55%, at least 58%, at least 60%, at least 65%, at least 70%, at least 75%, at least 78%, at least 80%, at least 83%, at least 88%, at least 90%, at least 91%, at least 92%, at least 93%, at least 94%, at least 95%, at least 96%, at least 97%, at least 98%, at least 99%, or 100% homology or identity to SEQ ID NO: 34, or an amino acid sequence least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology or identity to SEQ ID NO: 6. In some embodiments, the isolated UGT polypeptide catalyzing branching glucosylation of mogroside IV (M4) to mogroside V (M5) comprises an amino acid sequence having a homology or identity in the range of 89-100%, 90-100%, 92-85%, 94-80%, 95-100%, 96-100%, 97-100% or 99-100% to SEQ ID NO: 38, or 84-100%, 86-100%, 88-100%, 85-95%, 89-100%, 90-100%, 92-85%, 94-86%, 95-100%, 96-100%, 97-100% or 99-100% to SEQ ID NO: 6, or in the range of 34-100%, 40-90%, 37-85%, 45-80%, 50-75%, 55-65%, 80-90%, 93-100% to SEQ ID NO: 34. In a specific embodiment, the isolated UGT polypeptide catalyzing branching glucosylation of mogroside IV (M4) to mogroside V (M5) comprises an amino acid sequence as set forth in SEQ ID NO:38 or SEQ ID NO: 6 or SEQ ID NO:34. In some cases, SEQ ID NO:6 is also referred to as UGT94A9, A09 or UGT94-289-1.

In some embodiments, the UTG enzyme polypeptide catalyzes the branched glucosylation of C3 or C24 of mogroside or mogrol at the (1-2) and/or (1-6) position. However, it will be appreciated that, in some embodiments, the UGT enzyme polypeptides of the invention can comprise glucosylation activity at the (1-4) position as well.

According to some embodiments of some aspects of the invention, the enzyme polypeptides are enzymes catalyzing synthesis of mogrol, namely squalene synthase, squalene epoxidase, cucurbitadienol synthase, epoxide hydrolase (also known as epoxy hydratase) and cytochrome p450.

Thus, according to some aspects of the invention there is provided an isolated squalene epoxidase (SQE, also referred to as SE) polypeptide comprising an amino acid sequence at least 94% identical to SEQ ID NO: 14 or 89% identical to SEQ ID NO: 16, wherein the polypeptide catalyzes diepoxysqualene synthesis from squalene or oxidosqualene. In some embodiments, the squalene epoxidase (SQE) polypeptide comprises an amino acid sequence at least 94, at least 95%, at least 96%, at least 97%, at least 98%, at least 99%, or 100% homologous or identical to SEQ ID NO: 14, or at least 89%, at least 90%, at least 91%, at least 92%, at least 93%, at least 94%, at least 95%, at least 96%, at least 97%, at least 98%, at least 99%, or 100% homologous or identical to SEQ ID NO: 16. In some embodiments, the isolated SQE polypeptide comprises an amino acid sequence having a homology or identity in the range of 95-100%, 96-100%, 97-100% or 99-100% to SEQ ID NO: 14, or 89-100%, 90-100%, 92-100%, 93-100%, 94-100%, 95-100%, 96-100%, 97-100% or 99-100% to SEQ ID NO: 16. In a specific embodiment, the isolated SQE polypeptide catalyzing diepoxysqualene synthesis from squalene or oxidosqualene comprises an amino acid sequence as set forth in SEQ ID NO: 14 or SEQ ID NO: 16. In some cases, SEQ ID NO: 14 is also referred to as SE1, SQE1 and contig 18561. In some cases, SEQ ID NO: 14 is also referred to as SE2, SQE2 and contig 16760.

According to some aspects of the invention there is provided an isolated epoxide hydrolase (EH, EPH) polypeptide comprising an amino acid sequence at least 75% identical to SEQ ID NO: 18, SEQ ID NO: 22 or SEQ ID NO: 24, the polypeptide catalyzing 3, 24, 25 trihydroxy cucurbitadienol synthesis from 3-hydroxy, 24-25 epoxy cucurbitadienol. In some embodiments, the epoxide hydrolase (EH) polypeptide comprises an amino acid sequence at least 75%, at least 78%, at least 80%, at least 83%, at least 88%, at least 90%, at least 91%, at least 92%, at least 93%, at least 94%, at least 95%, at least 96%, at least 97%, at least 98%, at least 99%, or 100% homologous or identical to SEQ ID NO: 18, SEQ ID NO: 22 or SEQ ID NO: 24. In some embodiments, the isolated EH polypeptide comprises an amino acid sequence having a homology or identity in the range of 75-100%, 78-97%, 80-95%, 85-92%, 87-98%, 90-99%, 92-100%, 95-100%, 96-100%, 97-100% or 99-100% to SEQ ID NO: 18, or 22 or 24. In a specific embodiment, the isolated EH polypeptide catalyzing 3, 24, 25 trihydroxy cucurbitadienol synthesis from 3-hydroxy, 24-25 epoxy cucurbitadienol comprises the amino acid sequence as set forth in SEQ ID NO:18 or SEQ ID NO: 22 or SEQ ID NO: 24. In some cases, SEQ ID NO: 18 is also referred to as EH1, EPH1 and contig 73966. In some cases, SEQ ID NO: 22 is also referred to as EH3, EPH3 and contig 102640. In some cases, SEQ ID NO: 24 is referred to as EH4, EPH4 and contig 28382.

The UGT, SQE and EH enzyme polypeptides of the invention, having the indicated catalytic activity, can include UGT, SQE and EH enzyme polypeptides of any organism, having the indicated catalytic activity. In some embodiments isolated UGT, SQE or EH polypeptide is a plant UGT, SQE or EH polypeptide. In some embodiments, the plant is a plant of the Cucurbitaceae family. A detailed, non-limiting list of members of the Cucurbitaceae family is found below. In specific embodiments, the isolated UGT polypeptide is a Siraitia grosvenorii UGT, SQE or EH polypeptide. As used herein, the phrase “mogrol precursors” or “mogrol pathway precursors”, “mogrol precursor”, “mogrol precursor substrate” refers to at least squalene, monoepoxy squalene, diepoxy squalene, 3 hydroxy, 24-25 epoxy cucurbitadienol, 3, 11 dihydroxy 24-25 epoxy cucurbitadienol, 3, 24, 25 trihydroxy cucurbitadienol. It will be appreciated that, since mogrol is the substrate for mogroside synthesis, mogrol precursors (precursor substrates, mogrol pathway precursors) also constitute mogroside pathway precursors/substrates.

As used herein, the phrase “mogrol pathway enzymes” refers to at least a squalene epoxidase or at least 89% homologous or identical thereto capable of catalyzing diepoxy squalene synthesis from squalene, or at least a cucurbitadienol synthetase or 60% homologous or identical thereto, capable of catalyzing 3 hydroxy, 24-25 epoxy cucurbitadienol synthesis from diepoxy squalene, at least an epoxy hydratase or 75% homologous or identical thereto capable of catalyzing 3, 24, 25 trihydroxy cucurbitadienol synthesis from 3-hydroxy, 24-25 epoxy cucurbitadienol, and a Cytochrome P 450 enzyme or 60% homologous or identical thereto capable of catalyzing 3, 11, 24, 25 tetrahydroxy cucurbitadienol synthesis from 3, 24, 25 trihydroxy cucurbitadienol. (SQE: squalene epoxidase, EC:1.14.99.7; CS: cucurbitadienol synthase, EC:5.4.99.8; P450: cytochrome P450, EC:1.14.-.-.; and UDPG: UDP-glucosyltransferase, EC:2.4.1. E.C. 2.4.1 are UGTs)

As used herein, the term “mogroside pathway enzyme” refers to at least one or more uridine diphospho-glucosyl transferase (UGT) enzyme which catalyzes the glucosylation of a mogrol (un-glucosylated) or mogroside substrate.

Table 1 below comprises a non-limiting list of some mogrol and mogroside pathway enzymes useful in the methods and compositions of the present invention, including examples of homologues which can be suitable for use in some of the embodiments of the invention.

TABLE 1
MOGROL/MOGROSIDE PATHWAY ENZYMES

| | | | | ALSO |
|---|---|---|---|---|
| | SEQ ID NO: | | | REFERRED |
| ENZYME | DNA | PROT | CLOSEST HOMOLOG | TO AS |
| CDS | | | | |
| cucurbitadienol synthase | | | | |
| >SgCDS | 11 | 12 | cucurbitadienol synthase | |
| | | | [Siraitia grosvenorii] | |
| | | | gb\|AEM42982.1\| | |
| | | | SEQ ID NO: 45 | |
| CYP | | | | |
| cytochrome P450 | | | | |
| >Sg_cyp102801 | 9 | 10 | cytochrome P450 | CYP801 |
| | | | [Siraitia grosvenorii] | |
| | | | gb\|AEM42986.1\| | |
| | | | SEQ ID NO: 52 | |
| SQE | | | | |
| Squalene Epoxidase | | | | |
| >SQE18561p | 13 | 14 | squalene monooxygenase-like | SE1, SQE1, |
| | | | [Cucumis melo] | contig 18561 |
| | | | ref\|XP_008452686.1\| | |
| | | | SEQ ID NO: 46 | |
| >SQE16760p | 15 | 16 | squalene monooxygenase | SE2, SQE2, |
| | | | [Cucumis sativus] | Contig16760 |
| | | | ref\|XP_004142907.1\| | |
| | | | SEQ ID NO: 47 | |
| EPH | | | | |
| Epoxide hydrolase | | | | |
| >EPH73966p | 17 | 18 | bifunctional epoxide hydrolase | Epoxide |
| | | | 2-like [Cucumis sativus] | Hydratase, |
| | | | ref\|XP_004152243.1 | EH1, EPH1, |
| | | | SEQ ID NO: 48 | Contig73966 |
| >EPH86123p | 19 | 20 | bifunctional epoxide hydrolase | Epoxide |
| | | | 2-like isoform X1 | Hydratase, |
| | | | [Cucumis melo] | EH2, EPH2, |
| | | | ref\|XP_008454322.1 | Contig86123 |
| | | | SEQ ID NO: 49 | |
| >EPH102640 | 21 | 22 | bifunctional epoxide hydrolase | Epoxide |
| | | | 2-like [Cucumis melo] | Hydratase, |
| | | | ref\|XP_008454327.1\| | EH3, EPH3, |
| | | | SEQ ID NO: 50 | Contig 102640 |
| >EPH28382p | 23 | 24 | bifunctional epoxide hydrolase | Epoxide |
| | | | 2-like [Cucumis sativus] | Hydratase, |
| | | | ref\|XP_004152361.1\| | EH4, EPH4, |
| | | | SEQ ID NO: 51 | Contig28382 |
| UGT | | | | |
| Uridine | | | | |
| diphosphoglucosyl | | | | |
| transferase | | | | |
| >UGT73-251_5 | 25 | 26 | UDP-glycosyltransferase | UDPGT |
| | | | 73C3-like [Cucumis melo] | |
| | | | ref\|XP_008442743.1\| | |
| | | | SEQ ID NO: 53 | |
| >UGT73-251-6 | 27 | 28 | UDP-glycosyltransferase | UDPGT |
| | | | 73C3-like [Cucumis melo] | |
| | | | ref\|XP_008442743.1\| | |
| | | | SEQ ID NO: 53 | |
| >UGT73-348-2 | 3 | 4 | UDP-glycosyltransferase | UGT73E8, |
| | | | 73D1-like [Cucumis melo] | EO8, |
| | | | ref\|XP_008462511.1 | UDPGT |
| | | | SEQ ID NO: 54 | |
| >UGT73-327-2 | 7 | 8 | UDP-glucose flavonoid 3-O- | UGT73E7, |
| | | | glucosyltransferase 7-like | EO7, |
| | | | [Cucumis sativus] | UDPGT |
| | | | ref\|XP_004140708.1\| | |
| | | | SEQ ID NO: 55 | |
| >UGT74-345-2 | 1 | 2 | UDP-glycosyltransferase | UGT74B2, B02 |
| | | | 74E2-like [Cucumis melo] | UDPGT |
| | | | ref\|XP_008445481.1 | |
| | | | SEQ ID NO: 56 | |
| >UGT75-281-2 | 29 | 30 | crocetin glucosyltransferase, | 75 contig |
| | | | chloroplastic-like | 103243, E8, |
| | | | [Cucumis sativus] | UGT75nE8 |
| | | | ref\|XP_004140604.2 | UDPGT |
| | | | SEQ ID NO: 57 | |
| >UGT85-269-4 | 31 | 32 | 7-deoxyloganetic acid | UGT85E6, |
| | | | glucosyltransferase-like | UDPGT |
| | | | [Cucumis sativus] | |
| | | | ref\|XP_004147933.2 | |
| | | | SEQ ID NO: 58 | |
| >UGT85-269-1 | 33 | 34 | 7-deoxyloganetic acid | UGT85E5, |
| | | | glucosyltransferase-like | 85E5 |
| | | | [Cucumis sativus] | UDPGT |
| | | | ref\|XP_004147933.2\| | |
| | | | SEQ ID NO: 58 | |
| >UGT94-289-1 | 5 | 6 | beta-D-glucosyl crocetin beta- | UGT94A9, |
| | | | 1,6-glucosyltransferase-like | A09, |
| | | | [Cucumis sativus] | UDPGT |
| | | | ref\|XP_004142256.1 | |
| | | | SEQ ID NO: 59 | |
| >UGT94-289-2 | 35 | 36 | beta-D-glucosyl crocetin beta- | UGT9498, |
| | | | 1,6-glucosyltransferase-like | UDPGT |
| | | | [Cucumis sativus] | |
| | | | ref\|XP_004142256.1 | |
| | | | SEQ ID NO: 59 | |
| >UGT94-289_3 | 37 | 38 | beta-D-glucosyl crocetin beta- | UGT94C9, |
| | | | 1,6-glucosyltransferase-like | UDPGT |
| | | | [Cucumis sativus] | |
| | | | ref\|XP_004142256.1 | |
| | | | SEQ ID NO: 59 | |

As used herein the term “mogrol” refers to the aglycone compound mogrol.

Glycosylated mogrol or mogroside refers to a mogrol having at least one primary glucose or branched glucose at positions 3, 24 and/or 25. According to a specific embodiment, the glycosylated or glucosylated mogrol or mogroside refers to a mogrol having at least one primary glucose or branched glucose at positions 3 and/or 24.

The UGT enzyme polypeptides of the present invention can catalyze primary glucosylation and/or branching glucosylation of the mogrol or mogroside substrates. As used herein, the term “primary glucosylation” refers to covalent addition of a glucose moiety to an un-glucosylated carbon of the mogrol or mogroside substrate, resulting in a mono-glucosylated (M1) (when substrate is an aglycol mogrol) or di-glucosylated (when substrate is a mono-glucosylated mogroside) mogroside (M2). Glucosylations are typically at the C3 and C24 carbons of the mogrol backbone.

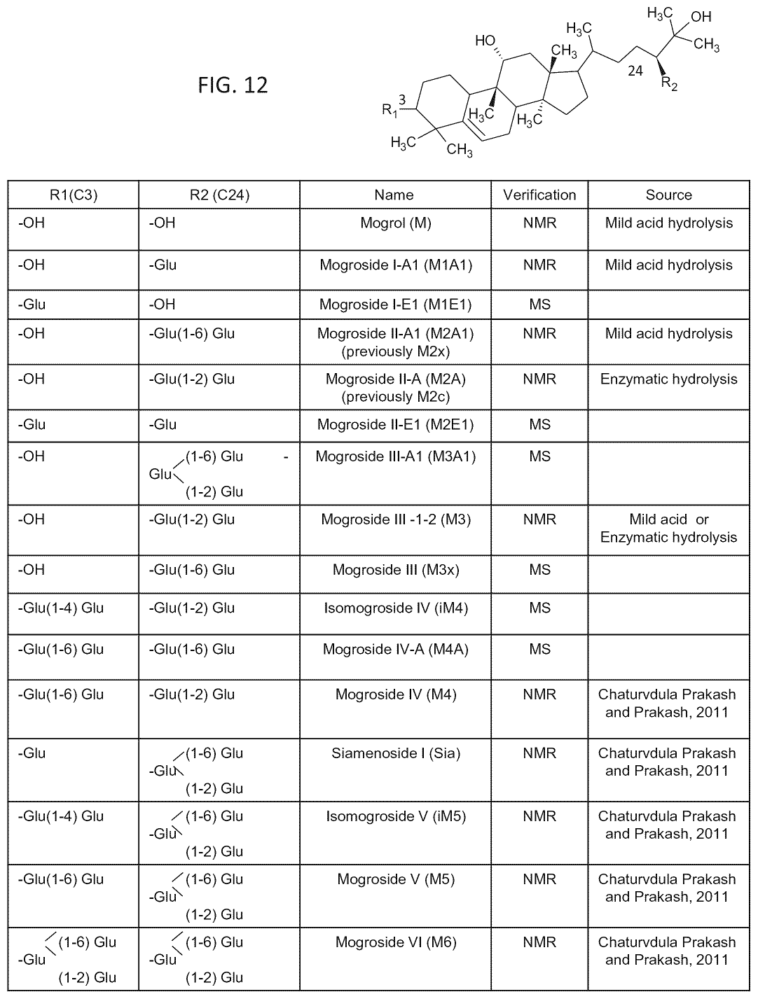

As used herein, the term “branching glucosylation” or “branched glucosylation” refers to the covalent addition of a glucose moiety to a glucose of a glucosylated carbon of a mogroside substrate, resulting in a multi-glucosylated mogroside (M2, M3, M4, M5 or M6), depending on the level of glucosidation of the mogroside substrate. Glucosylations are typically at the C3 and C24 carbons of the mogrol backbone. A table illustrating a non-limiting number of unglucosylated mogrol and different forms of mogroside, glucosylated at different carbons, and with different linkages, is shown in FIG. 12.

The mogrol biosynthetic pathway enzyme and mogroside biosynthetic pathway enzyme polypeptides of the invention can be used to synthesize a mogrol, mogrol precursor or mogroside or mogroside precursor.

Thus, according to some embodiments of some aspects of the invention there is provided a method of synthesizing a mogrol or mogrol precursor product from a mogrol precursor substrate, the method comprising contacting at least one mogrol precursor substrate with a mogroside pathway enzyme. The mogroside pathway enzymes catalyzing the steps of mogrol, mogroside or mogrol or mogroside precursor biosynthesis can be as follows:
(a) when the mogrol precursor product comprises diepoxy squalene and the enzyme comprises a squalene epoxidase polypeptide as described herein, thereby producing diepoxy squalene. Squalene epoxidase polypeptides of the invention suitable for use in the method include SQE polypeptides comprising SEQ ID NO: 14, or at least 94% identical or homologous thereto, or SEQ ID NO: 16 or at least 89% identical or homologous thereto, or
(b) when the mogrol precursor product comprises 3 hydroxy, 24-25 epoxy cucurbitadienol and the mogrol precursor substrate comprises diepoxy squalene, the mogrol pathway enzyme comprises a cucurbitadienol synthetase polypeptide as set forth in SEQ ID NO: 12 or 60% homologous or identical thereto, thereby producing a 3 hydroxy, 24-25 epoxy cucurbitadienol, or
(c) when the product comprises 3, 24, 25 trihydroxy cucurbitadienol and the substrate comprises 3-hydroxy, 24-25 epoxy cucurbitadienol, the mogrol pathway enzyme comprises an epoxy hydratase polypeptide as described in any one of claims 21-23, thereby producing a 3, 24, 25 trihydroxy cucurbitadienol. Epoxy hydratase (also known as epoxide hydrolase) polypeptides of the invention suitable for use in the method include EH polypeptides comprising SEQ ID NO: 18, 22 or 24 or at least 75% identical or homologous thereto, or
(d) when the product comprises mogrol and the mogrol precursor substrate comprises 3, 24, 25 trihydroxy cucurbitadienol, the mogrol pathway enzyme is Cytochrome P 450 enzyme as set forth in SEQ ID NO: 10 or 60% homologous or identical thereto, thereby producing 3, 11, 24, 25 tetrahydroxy cucurbitadienol (mogrol).

Biosynthesis of the mogrol or mogroside can be reconstituted in a cell expressing one or more of the mogroside biosynthesis enzyme polypeptides of the invention. Depending upon the availability of mogrol precursors and biosynthetic enzymes in the cell (or cell lysate), the individual reactions, or combinations thereof can be reconstituted using any one of, some of or all of the steps described above. Thus, in some embodiments, producing the mogrol product comprises at least one of the steps of:
(i) contacting the squalene or oxido squalene with a squalene epoxidase enzyme polypeptide of the invention, thereby producing diepoxy squalene;
(ii) contacting the diepoxy squalene with a cucurbitadienol synthase of the invention, thereby producing 3 hydroxy, 24-25 epoxy cucurbitadienol;
(iii) contacting the 3 hydroxy, 24-25 epoxy cucurbitadienol with an epoxy hydratase (epoxide hydrolase) enzyme of the invention, thereby producing 3, 24, 25 trihydroxy cucurbitadienol; and
(iv) contacting the 3, 24-25 trihydroxy cucurbitadienol with a Cytochrome P 450 enzyme of the invention, thereby producing the mogrol product (3, 11, 24, 25 tetrahydroxy cucurbitadienol).

In some embodiments, producing the mogrol product comprises at least (i) and (iv), at least (ii) and (iv), at least (iii) and (iv), at least (i), (ii) and (iii), at least (i), (ii) and (iv), at least (i), (iii) and (iv), at least (ii), (iii) and (iv), and optionally all of (i) (ii), (iii) and (iv). For example, in order to reconstitute or enhance dioxidosqualene synthesis in a cell lacking or deficient in squalene epoxidase, but having the biosynthetic capabilities for completing the synthesis of mogrol from dioxidosqualene, the method can comprise (i). In a cell capable of synthesizing dioxidosqualene, 3 hydroxy, 24-25 epoxy cucurbitadienol, and 3, 24-25 trihydroxy cucurbitadienol, but deficient or lacking in epoxide hydrolase (epoxy hydratase), the method can comprise (iii). In a cell capable of synthesizing 3 hydroxy, 24-25 epoxy cucurbitadienol, and 3, 24-25 trihydroxy cucurbitadienol, but deficient or lacking in squalene epoxidase and epoxide hydrolase (epoxy hydratase), the method can comprise (i) and (iii).

The present invention contemplates mogroside biosynthesis. According to some embodiments of some aspects of the invention there is provided a method of synthesizing a mogroside, the method comprising contacting at least one UGT polypeptide of the invention or a combination thereof with at least one UGT substrate mogroside precursor.

According to some embodiments, the method comprises the steps of primary and branching glucosylation of the mogrol or mogroside precursor substrates. The mogroside pathway enzymes catalyzing the steps of mogroside or mogroside precursor biosynthesis can be as follows:
(aa) When the substrate is mogrol, or mogroside un-glucosylated at C3, the UGT catalyzing primary glucosylation of mogrol at C24 and primary glucosylation of mogroside at C3 is a UGT comprising an amino acid sequence set forth in SEQ ID NO: 34 or at least 34% homologous or identical thereto.
(bb) When the substrate is a mogroside, the UGT catalyzing branching glucosylation of mogroside at the (1-2) and (1-6) positions of C3 and/or branching glucosylation of mogroside at the (1-2) and (1-6) positions of C24 comprises an amino acid sequence as set forth in SEQ ID NO: 38 or at least 89% homologous or identical thereto.
(cc) When the substrate is a mogroside M5, the UGT catalyzing branching glucosylation of mogroside M5 to mogroside M6 comprises an amino acid sequence as set forth in SEQ ID NO: 38 or at least 89% homologous or identical thereto, or SEQ ID NO: 8, or at least 41% homologous or identical thereto.
(dd) When the substrate is a mogroside IV (M4), the UGT catalyzing branching glucosylation of M4 to mogroside V (M5) comprises an amino acid sequence as set forth in any one of SEQ ID NO: 38, or at least 89% homologous or identical thereto, SEQ ID NO: 34, or at least 34% homologous or identical thereto, and SEQ ID NO: 6, or at least 84% homologous or identical thereto.

Thus, in some embodiments, the method comprises contacting the mogroside substrate with at least one UGT polypeptide selected from the group comprising an amino acid sequence as set forth in SEQ ID NO: 38, or at least 89% homologous or identical thereto, SEQ ID NO: 34, or at least 34% homologous or identical thereto, SEQ ID NO: 8, or at least 41% homologous or identical thereto and SEQ ID NO: 6, or at least 84% homologous or identical thereto.

In some embodiments, producing the mogroside product comprises at least (aa) and (bb), at least (aa) and (cc), at least (aa) and (dd), at least (aa), (bb) and (cc), at least (aa), (cc) and (dd), at least (bb), (cc) and (dd), at least (bb) and (cc), at least (cc) and (dd), and optionally all of (aa) (bb), (cc) and (dd). For example, in order to reconstitute or enhance mogroside synthesis in a cell lacking or deficient in UGT catalyzing primary glucosylation at C3 or C24, but having the biosynthetic capabilities for completing the synthesis of mogroside from mono-glucosylated mogroside, the method can comprise (aa). In a cell capable of synthesizing M5, but deficient or lacking in UGT catalyzing branching glucosylation of M5 to M6, the method can comprise (cc). In a cell capable of having the biosynthetic capabilities for completing the synthesis of mogroside M5 from mono-glucosylated mogroside, but deficient or lacking in primary glucosylation of C3 or C24 and in branching glucosylation of M5 to M6, the method can comprise (aa) and (cc). In some embodiments, the method comprises contacting the mogroside substrate with at least a UGT polypeptide comprising an amino acid sequence as set forth in SEQ ID NO: 34, or at least 34% homologous or identical thereto and one or more of a UGT polypeptide comprising an amino acid sequence as set forth in SEQ ID NO: 8, or at least 41% homologous or identical thereto, a UGT polypeptide comprising an amino acid sequence as set forth in SEQ ID NO: 6 or 84% homologous or identical thereto, and a UGT polypeptide comprising an amino acid sequence as set forth in SEQ ID NO: 38, or 89% homologous or identical thereto. In a specific embodiment, the method comprises contacting the mogroside substrate with at least a UGT polypeptide comprising an amino acid sequence as set forth in SEQ ID NO: 34, or at least 34% homologous or identical thereto and a UGT polypeptide comprising an amino acid sequence as set forth in SEQ ID NO: 38, or 89% homologous or identical thereto.

The present invention contemplates mogroside biosynthesis from mogrol substrates and/or precursors. Thus, the methods of the invention for synthesizing a mogroside comprises combining producing a mogrol according to a method of the invention, and synthesizing the mogroside as described hereinabove, i.e. combining any one or more, or all of the steps of the mogrol synthesis described herein with any one or more, or all of the steps of the mogroside synthesis described herein.

Production of all possible mogroside products is contemplated. Thus, in some embodiments, the mogroside is selected from the group consisting of mogroside I-A1, mogroside I-E1, mogroside IIE, mogroside III, siamenoside, mogroside V and mogroside VI.

According to some embodiments, the method further comprises isolating the mogroside. Methods for isolation and purification of mogroside compounds are well known in the art, for example, Li, D. et al J. Nat. Med. 2007, 61, 307-312.; Venkata Chaturvedula and Indra Prakash., J. Carb. Chem. 2011 30, 16-26.; Venkata Sai Prakash Chaturvedula, Indra Prakash. IOSR Journal of Pharmacy (IOSRPHR) 2012. 2, 7-12.

As used herein, the term “polypeptide” refers to a linear organic polymer consisting of a large number of amino-acid residues bonded together by peptide bonds in a chain, forming part of (or the whole of) a protein molecule. The amino acid sequence of the polypeptide refers to the linear consecutive arrangement of the amino acids comprising the polypeptide, or a portion thereof.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

The term “isolated” refers to at least partially separated from the natural environment e.g., from a plant cell.

As used herein “expressing” refers to expression at the mRNA and optionally polypeptide level.

As used herein, the phrase “exogenous polynucleotide” refers to a heterologous nucleic acid sequence which may not be naturally expressed within the plant (e.g., a nucleic acid sequence from a different species) or which overexpression in the plant is desired. The exogenous polynucleotide may be introduced into the plant in a stable or transient manner, so as to produce a ribonucleic acid (RNA) molecule and/or a polypeptide molecule. It should be noted that the exogenous polynucleotide may comprise a nucleic acid sequence which is identical or partially homologous to an endogenous nucleic acid sequence of the plant.

The term “endogenous” as used herein refers to any polynucleotide or polypeptide which is present and/or naturally expressed within a plant or a cell thereof.

Homologous sequences include both orthologous and paralogous sequences.

The term “paralogous” relates to gene-duplications within the genome of a species leading to paralogous genes. The term “orthologous” relates to homologous genes in different organisms due to ancestral relationship. Thus, orthologs are evolutionary counterparts derived from a single ancestral gene in the last common ancestor of given two species and therefore have great likelihood of having the same function.

One option to identify orthologues in monocot plant species is by performing a reciprocal BLAST search. This may be done by a first blast involving blasting the sequence-of-interest against any sequence database, such as the publicly available NCBI database which may be found at: ncbi(dot)nlm(dot)nih(dot)gov. If orthologues in rice were sought, the sequence-of-interest would be blasted against, for example, the 28,469 full-length cDNA clones from Oryza sativa Nipponbare available at NCBI. The blast results may be filtered. The full-length sequences of either the filtered results or the non-filtered results are then blasted back (second blast) against the sequences of the organism from which the sequence-of-interest is derived. The results of the first and second blasts are then compared. An orthologue is identified when the sequence resulting in the highest score (best hit) in the first blast identifies in the second blast the query sequence (the original sequence-of-interest) as the best hit. Using the same rational a paralogue (homolog to a gene in the same organism) is found. In case of large sequence families, the ClustalW program may be used [ebi(dot)ac(dot)uk/Tools/clustalw2/index(dot)html], followed by a neighbor-joining tree (wikipedia(dot)org/wiki/Neighbor-joining) which helps visualizing the clustering.

Homology (e.g., percent homology, sequence identity+sequence similarity) can be determined using any homology comparison software computing a pairwise sequence alignment.

As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff JG. [Amino acid substitution matrices from protein blocks. Proc. Natl. Acad. Sci. U.S.A. 1992, 89(22): 10915-9].

Identity (e.g., percent homology) can be determined using any homology comparison software, including for example, the BlastN software of the National Center of Biotechnology Information (NCBI) such as by using default parameters.

According to some embodiments of the invention, the identity is a global identity, i.e., an identity over the entire amino acid or nucleic acid sequences of the invention and not over portions thereof.

According to some embodiments of the invention, the term “homology” or “homologous” refers to identity of two or more nucleic acid sequences; or identity of two or more amino acid sequences; or the identity of an amino acid sequence to one or more nucleic acid sequence.

According to some embodiments of the invention, the homology is a global homology, i.e., an homology over the entire amino acid or nucleic acid sequences of the invention and not over portions thereof.

The degree of homology or identity between two or more sequences can be determined using various known sequence comparison tools which are described in WO2014/102774.

Local alignments tools, which can be used include, but are not limited to, the tBLASTX algorithm, which compares the six-frame conceptual translation products of a nucleotide query sequence (both strands) against a protein sequence database. Default parameters include: Max target sequences: 100; Expected threshold: 10; Word size: 3; Max matches in a query range: 0; Scoring parameters: Matrix-BLOSUM62; filters and masking: Filter-low complexity regions.

Microorganisms, plant cells, or plants can be developed that express polypeptides useful for the biosynthesis of mogrol (the triterpene core) and various mogrol glycosides (mogrosides). The aglycone mogrol is glycosylated with different numbers of glucose moieties to form various mogroside compounds.

In general, the method of producing a mogroside may be performed either in vitro or in vivo. It is also comprised within the invention that some steps are performed in vitro, whereas others may be performed in vivo. Thus, for example the first steps may be performed in vitro and where after an intermediate product may be fed to recombinant host cells, capable of performing the remaining steps of the method. Alternatively, the first steps may be performed in vivo and where after an intermediate product may be used as substrate for the subsequent step(s) performed in vitro. Other combinations can also be envisaged. When the methods are performed in vitro each of the steps of the methods may be performed separately. Alternatively, one or more of the steps may be performed within the same mixture. In embodiments wherein some or all of the steps of the methods are performed separately, then the intermediate product of each of the steps may be purified or partly purified before performing the next step.

When the methods are performed in vivo, the methods employ use of a recombinant host expressing one or more of the enzymes or the methods may employ use of several recombinant hosts expressing one or more of the enzymes.

The present invention contemplates the recombinant production of mogrol, or morgoside. Thus, in some embodiments, the method of mogrol and/or mogroside biosynthesis is performed in a recombinant cell exogenously expressing at least one of the SQE, CDS, EH, Cyt p450 and UGT enzyme polypeptides of the invention. In some embodiments, the recombinant cell expresses at least one enzyme polypeptide selected from the group consisting of a UGT polypeptide comprising the amino acid sequence as set forth in SEQ ID NO: 34 or at least 34% identical or homologous thereto, a UGT polypeptide comprising the amino acid sequence as set forth in SEQ ID NO: 6 or at least 84% identical or homologous thereto, a UGT polypeptide comprising the amino acid sequence as set forth in SEQ ID NO: 38 or at least 89% identical or homologous thereto, a SQE polypeptide comprising the amino acid sequence as set forth in SEQ ID NO: 14 or at least 94% identical or homologous thereto, or SEQ ID NO: 16 or at least 89% identical or homologous thereto, and an EH polypeptide comprising the amino acid sequence as set forth in any one of SEQ ID NOs: 18, 22 or 24 or at least 75% identical or homologous thereto.

Recombinant expression of the polypeptides of the invention, or recombinant production of mogrol substrates, mogrol and/or mogroside compounds can be performed in a host cell expressing an isolated polynucleotide comprising a nucleic acid sequence encoding the isolated polypeptide of the mogrol and or mogroside biosynthetic pathway enzyme of the invention. In some embodiments, the isolated polynucleotide is provided in a nucleic acid construct useful in transforming the host cell. Suitable host cells include bacteria, yeast and other microorganisms that can be cultured or grown in fermentation, plant and other eukaryotic cells. In some embodiments, the nucleic acid construct of some embodiments of the invention can be utilized to transform plant cells.

Isolated polynucleotides suitable for use with the methods of the invention include, but are not limited to, polynucleotides encoding any of the mogrol and mogroside biosynthesis pathway enzymes as shown in Table 1. Thus, in some embodiments, there is provided an isolated polynucleotide comprising a nucleic acid sequence encoding the amino acid sequence as set forth in any one of SEQ ID NOs: 6, 10, 12, 14, 16, 22, 24, 34 and 38, or functional homologs thereof.

Functional homologs of the polypeptides described above are also suitable for use in the methods and recombinant hosts described herein. A functional homolog is a polypeptide that has sequence similarity to a reference polypeptide, and that carries out one or more of the biochemical or physiological function(s) of the reference polypeptide. Thus, functional homologues of the enzymes described herein are polypeptides that have sequence similarity to the reference enzyme, and which are capable of catalyzing the same step or part of a step of the methods of the invention as the reference enzyme. In general it is preferred that functional homologues share at least some degree of sequence identity with the reference polypeptide, for example, as indicated hereinabove for the UGT, SE, EH, CDS, Cyt p450 enzyme polypeptides of the invention.

According to some embodiments of the invention, the heterologous polynucleotide of the invention encodes a UGT polypeptide comprising the amino acid sequence as set forth in SEQ ID NO: 34 or at least 34% identical or homologous thereto, a UGT polypeptide comprising the amino acid sequence as set forth in SEQ ID NO: 6 or at least 84% identical or homologous thereto, a UGT polypeptide comprising the amino acid sequence as set forth in SEQ ID NO: 38 or at least 89% identical or homologous thereto, a SQE polypeptide comprising the amino acid sequence as set forth in SEQ ID NO: 14 or at least 94% identical or homologous thereto, or SEQ ID NO: 16 or at least 89% identical or homologous thereto, and an EH polypeptide comprising the amino acid sequence as set forth in any one of SEQ ID NOs: 18, 22 or 24 or at least 75% identical or homologous thereto.

In some embodiments, the isolated polynucleotide comprises a nucleic acid sequence selected from the group consisting of SEQ ID NOs. 5, 9, 11, 13, 15, 17, 21, 23, 33 and 37.

The term ““plant” as used herein encompasses whole plants, a grafted plant, ancestors and progeny of the plants and plant parts, including seeds, shoots, stems, roots (including tubers), rootstock, scion, and plant cells, tissues and organs. The plant may be in any form including suspension cultures, embryos, meristematic regions, callus tissue, leaves, gametophytes, sporophytes, pollen, and microspores. Plants that are particularly useful in the methods of the invention include all plants which belong to the superfamily Viridiplantee, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaca plurijuga, Betula spp., Brassica spp., Bruguiera gymnorrhiza, Burkea africana, Butca frondosa, Cadaba farinosa, Calliandra spp, Camellia sinensis, Canna indica, Capsicum spp., Cassia spp., Centrocma pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalypfus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phascolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, trees. Alternatively algae and other non-Viridiplantae can be used for the methods of some embodiments of the invention. In specific embodiments, the plant is a plant of the Cucurbitacae family, such as S. grosvenorii. In some embodiments, the plant cells expressing the polypeptides of the invention comprise fruit or root cells of a Cucurbitaceae plant.

The present invention contemplates the use of nucleic acid constructs for transformation of cells for expression of the mogroside biosynthesis pathway enzyme polypeptides and production of mogrol, mogrol precursors and mogroside. Thus, in some embodiments, there is provided a nucleic acid construct comprising an isolated polynucleotide of the invention and a cis-acting regulatory element for directing expression of the isolated polynucleotide.

Constructs useful in the methods according to some embodiments of the invention may be constructed using recombinant DNA technology well known to persons skilled in the art. The gene constructs may be inserted into vectors, which may be commercially available, suitable for transforming into plants and suitable for expression of the gene of interest in the transformed cells. The genetic construct can be an expression vector wherein the nucleic acid sequence is operably linked to one or more regulatory sequences allowing expression in the plant cells.

In a particular embodiment of some embodiments of the invention the regulatory sequence is a plant-expressible promoter.

As used herein the phrase “plant-expressible” refers to a promoter sequence, including any additional regulatory elements added thereto or contained therein, is at least capable of inducing, conferring, activating or enhancing expression in a plant cell, tissue or organ, preferably a monocotyledonous or dicotyledonous plant cell, tissue, or organ. Examples of preferred promoters useful for the methods of some embodiments of the invention are presented in Table 2, 3, 4 and 5.

TABLE 2
Exemplary constitutive promoters for use in the
performance of some embodiments of the invention

| Gene Source | Expression Pattern | Reference |
|---|---|---|
| Actin | constitutive | McElroy et al, Plant Cell, |
| | | 2: 163-171, 1990 |
| CAMV 35S | constitutive | Odell et al, Nature, |
| | | 313: 810-812, 1985 |
| CaMV 19S | constitutive | Nilsson et al., Physiol. |
| | | Plant 100: 456-462, 1997 |
| GOS2 | constitutive | de Pater et al, Plant J Nov; |
| | | 2(6): 837-44, 1992 |
| ubiquitin | constitutive | Christensen et al, Plant |
| | | Mol. Biol. 18: 675-689, |
| | | 1992 |
| Rice cyclophilin | constitutive | Bucholz et al, Plant Mol |
| | | Biol. 25(5): 837-43, 1994 |
| Maize H3 histone | constitutive | Lepetit et al, Mol. Gen. |
| | | Genet. 231: 276-285, 1992 |
| Actin 2 | constitutive | An et al, Plant J. 10(1); |
| | | 107-121, 1996 |

TABLE 3
Exemplary seed-preferred promoters for use in the
performance of some embodiments of the invention

| Gene Source | Expression Pattern | Reference |
|---|---|---|
| Seed specific genes | seed | Simon, et al., Plant Mol. Biol. |
| | | 5. 191, 1985; Scofield, |
| | | et al., J. Biol. Chem. 262: |
| | | 12202, 1987.; Baszczynski, |
| | | et al., Plant Mol. Biol. 14: |
| | | 633, 1990. |
| Brazil Nut albumin | seed | Pearson' et al., Plant Mol. |
| | | Biol. 18: 235-245, 1992. |
| legumin | seed | Ellis, et al. Plant Mol. Biol. |
| | | 10: 203-214, 1988 |
| Glutelin (rice) | seed | Takaiwa, et al., Mol. Gen. |
| | | Genet. 208: 15-22, 1986; |
| | | Takaiwa, et al., FEBS Letts. |
| | | 221: 43-47, 1987 |
| Zein | seed | Matzke et al Plant Mol Biol, |
| | | 143). 323-32 1990 |
| napA | seed | Stalberg, et al, Planta 199: |
| | | 515-519, 1996 |
| wheat LMW and | endosperm | Mol Gen Genet 216: 81-90, |
| HMW, glutenin-1 | | 1989; NAR 17: 461-2, |
| Wheat SPA | seed | Albanietal, Plant Cell, 9: |
| | | 171-184, 1997 |
| wheat a, b and g | endosperm | EMBO3: 1409-15, 1984 |
| gliadins | | |
| Barley ltrl promoter | endosperm | |
| barley B1, C, D | endosperm | Theor Appl Gen 98: 1253-62, |
| hordein | | 1999; Plant J 4: 343-55, |
| | | 1993; Mol Gen Genet 250: |
| | | 750-60, 1996 |
| Barley DOF | endosperm | Mena et al, The Plant |
| | | Journal, 116(1): 53-62, 1998 |
| Biz2 | endosperm | EP99106056.7 |
| Synthetic promoter | endosperm | Vicente-Carbajosa et al., |
| | | Plant J. 13: 629-640, 1998 |
| rice prolamin | endosperm | Wu et al, Plant Cell |
| NRP33 | | Physiology 39(8) 885-889, |
| | | 1998 |
| rice-globulin Glb-1 | endosperm | Wu et al, Plant Cell |
| | | Physiology 398) 885-889, |
| | | 1998 |
| rice OSH1 | emryo | Sato et al, Proc. Nati. Acad. |
| | | Sci. USA, 93: 8117-8122 |
| rice alpha-globulin | endosperm | Nakase et al. Plant Mol. Biol. |
| REB/OHP-1 | | 33: 513-S22, 1997 |
| rice ADP-glucose | endosperm | Trans Res 6: 157-68, 1997 |
| PP | | |
| maize ESR gene | endosperm | Plant J 12: 235-46, 1997 |
| family | | |
| sorgum gamma- | endosperm | PMB 32: 1029-35, 1996 |
| kafirin | | |
| KNOX | emryo | Postma-Haarsma ef al, Plant |
| | | Mol. Biol. 39: 257-71, 1999 |
| rice oleosin | Embryo and | Wu et at, J. Biochem., 123: |
| | aleuton | 386, 1998 |
| sunflower oleosin | Seed (embryo and | Cummins, et al., Plant Mol. |
| | dry seed) | Biol. 19: 873-876, 1992 |
| Tobacco NsCBTS | trichomes | Ennajdaoui et al., Plant Mol |
| | | Biol. 73: 6730685. 2010 |

TABLE 4
Exemplary flower-specific promoters
for use in the performance of the invention

| Gene Source | Expression Pattern | Reference |
|---|---|---|
| AtPRP4 | flowers | www(dot)salus(dot) |
| | | medium(dot)edu/mmg/ |
| | | tierney/html |
| chalene synthase | flowers | Van der Meer, et al., Plant |
| (chsA) | | Mol. Biol. 15, 95-109, 1990. |
| LAT52 | anther | Twell et al Mol. Gen Genet. |
| | | 217: 240-245 (1989) |
| apetala-3 | flowers | |

TABLE 5
Alternative rice promoters for use in the performance of the invention

| PRO # | gene | expression |
|---|---|---|
| PR00001 | Metallothionein Mte | transfer layer of |
| | | embryo + calli |
| PR00005 | putative beta-amylase | transfer layer of |
| | | embryo |
| PR00009 | Putative cellulose synthase | Weak in roots |
| PR00012 | lipase (putative) | |
| PR00014 | Transferase (putative) | |
| PR00016 | peptidyl prolyl cis-trans | |
| | isomerase (putative) | |
| PR00019 | unknown | |
| PR00020 | prp protein (putative) | |
| PR00029 | noduline (putative) | |
| PR00058 | Proteinase inhibitor Rgpi9 | seed |
| PR00061 | beta expansine EXPB9 | Weak in young flowers |
| PR00063 | Structural protein | young tissues + calli + |
| | | embryo |
| PR00069 | xylosidase (putative) | |
| PR00075 | Prolamine 10Kda | strong in endosperm |
| PR00076 | allergen RA2 | strong in endosperm |
| PR00077 | prolamine RP7 | strong in endosperm |
| PR00078 | CBP80 | |
| PR00079 | starch branching enzyme I | |
| PR00080 | Metallothioneine-like ML2 | transfer layer of |
| | | embryo + calli |
| PR00081 | putative caffeoyl-CoA 3-0 | shoot |
| | methyltransferase | |
| PR00087 | prolamine RM9 | strong in endosperm |
| PR00090 | prolamine RP6 | strong in endosperm |
| PR00091 | prolamine RP5 | strong in endosperm |
| PR00092 | allergen RA5 | |
| PR00095 | putative methionine | embryo |
| | aminopeptidase | |
| PR00098 | ras-related GTP binding | |
| | protein | |
| PR00104 | beta expansine EXPB1 | |
| PR00105 | Glycine rich protein | |
| PR00108 | metallothionein like protein | |
| | (putative) | |
| PR00110 | RCc3 strong root | |
| PR00111 | uclacyanin 3-like protein | weak discrimination |
| | | center/shoot meristem |
| PR00116 | 26S proteasome regulatory | very weak meristem |
| | particle non-ATPase | specific |
| | subunit 11 | |
| PR00117 | putative 40S ribosomal protein | weak in endosperm |
| PR00122 | chlorophyll a/lo-binding | very weak in shoot |
| | protein precursor (Cab27) | |
| PR00123 | putative protochlorophyllide | Strong leaves |
| | reductase | |
| PR00126 | metallothionein RiCMT | strong discrimination |
| | | center shoot meristem |
| PR00129 | GO52 | Strong constitutive |
| PR00131 | GO59 | |
| PR00133 | chitinase Cht-3 | very weak meristem |
| | | specific |
| PR00135 | alpha-globulin | Strong in endosperm |
| PR00136 | alanine aminotransferase | Weak in endosperm |
| PR00138 | Cyclin A2 | |
| PR00139 | Cyclin D2 | |
| PR00140 | Cyclin D3 | |
| PR00141 | Cyclophyllin 2 | Shoot and seed |
| PR00146 | sucrose synthase SS1 (barley) | medium constitutive |
| PR00147 | trypsin inhibitor ITR1 (barley) | weak in endosperm |
| PR00149 | ubiquitine 2 with intron | strong constitutive |
| PR00151 | WSI18 | Embryo and stress |
| PR00156 | HVA22 homologue (putative) | |
| PR00157 | EL2 | |
| PR00169 | aquaporine | medium constitutive |
| | | in young plants |
| PR00170 | High mobility group protein | Strong constitutive |
| PR00171 | reversibly glycosylated | weak constitutive |
| | protein RGP1 | |
| PR00173 | cytosolic MDH | shoot |
| PR00175 | RAB21 | Embryo and stress |
| PR00176 | CDPK7 | |
| PR00177 | Cdc2-1 | very weak in meristem |
| PR00197 | sucrose synthase 3 | |
| PRO0198 | OsVP1 | |
| PRO0200 | OSH1 | very weak in young |
| | | plant meristem |
| PRO0208 | putative chlorophyllase | |
| PRO0210 | OsNRT1 | |
| PRO0211 | EXP3 | |
| PRO0216 | phosphate transporter OjPT1 | |
| PRO0218 | oleosin 18kd | aleurone + embryo |
| PRO0219 | ubiquitine 2 without intron | |
| PRO0220 | RFL | |
| PRO0221 | maize UBI delta intron | not detected |
| PRO0223 | glutelin-1 | |
| PRO0224 | fragment of prolamin RP6 | |
| | promoter | |
| PRO0225 | 4xABRE | |
| PRO0226 | glutelin OSGLUA3 | |
| PRO0227 | BLZ-2_short (barley) | |
| PR00228 | BLZ-2_long (barley) | |

Nucleic acid sequences of the polypeptides of some embodiments of the invention may be optimized for plant expression. Examples of such sequence modifications include, but are not limited to, an altered G/C content to more closely approach that typically found in the plant species of interest, and the removal of codons atypically found in the plant species commonly referred to as codon optimization.

The phrase “codon optimization” refers to the selection of appropriate DNA nucleotides for use within a structural gene or fragment thereof that approaches codon usage within the plant of interest. Therefore, an optimized gene or nucleic acid sequence refers to a gene in which the nucleotide sequence of a native or naturally occurring gene has been modified in order to utilize statistically-preferred or statistically-favored codons within the plant. The nucleotide sequence typically is examined at the DNA level and the coding region optimized for expression in the plant species determined using any suitable procedure, for example as described in Sardana et al. (1996, Plant Cell Reports 15:677-681). In this method, the standard deviation of codon usage, a measure of codon usage bias, may be calculated by first finding the squared proportional deviation of usage of each codon of the native gene relative to that of highly expressed plant genes, followed by a calculation of the average squared deviation. The formula used is: 1 SDCU=n=1 N[(Xn−Yn)/Yn] 2/N, where Xn refers to the frequency of usage of codon n in highly expressed plant genes, where Yn to the frequency of usage of codon n in the gene of interest and N refers to the total number of codons in the gene of interest. A table of codon usage from highly expressed genes of dicotyledonous plants is compiled using the data of Murray et al. (1989, Nuc Acids Res. 17:477-498).

One method of optimizing the nucleic acid sequence in accordance with the preferred codon usage for a particular plant cell type is based on the direct use, without performing any extra statistical calculations, of codon optimization tables such as those provided on-line at the Codon Usage Database through the NIAS (National Institute of Agrobiological Sciences) DNA bank in Japan (www(dot)kazusa(dot)or(dot)jp/codon/). The Codon Usage Database contains codon usage tables for a number of different species, with each codon usage table having been statistically determined based on the data present in Genbank.

By using the above tables to determine the most preferred or most favored codons for each amino acid in a particular species (for example, rice), a naturally-occurring nucleotide sequence encoding a protein of interest can be codon optimized for that particular plant species. This is effected by replacing codons that may have a low statistical incidence in the particular species genome with corresponding codons, in regard to an amino acid, that are statistically more favored. However, one or more less-favored codons may be selected to delete existing restriction sites, to create new ones at potentially useful junctions (5′ and 3′ ends to add signal peptide or termination cassettes, internal sites that might be used to cut and splice segments together to produce a correct full-length sequence), or to eliminate nucleotide sequences that may negatively effect mRNA stability or expression.

The naturally-occurring encoding nucleotide sequence may already, in advance of any modification, contain a number of codons that correspond to a statistically-favored codon in a particular plant species. Therefore, codon optimization of the native nucleotide sequence may comprise determining which codons, within the native nucleotide sequence, are not statistically-favored with regards to a particular plant, and modifying these codons in accordance with a codon usage table of the particular plant to produce a codon optimized derivative. A modified nucleotide sequence may be fully or partially optimized for plant codon usage provided that the protein encoded by the modified nucleotide sequence is produced at a level higher than the protein encoded by the corresponding naturally occurring or native gene. Construction of synthetic genes by altering the codon usage is described in for example PCT Patent Application 93/07278.

Thus, some embodiments of the invention encompasses nucleic acid sequences described hereinabove; fragments thereof, sequences hybridizable therewith, sequences homologous thereto, sequences orthologous thereto, sequences encoding similar polypeptides with different codon usage, altered sequences characterized by mutations, such as deletion, insertion or substitution of one or more nucleotides, either naturally occurring or man induced, either randomly or in a targeted fashion.

Plant cells may be transformed stably or transiently with the nucleic acid constructs of some embodiments of the invention. In stable transformation, the nucleic acid molecule of some embodiments of the invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches:
(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.
(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by some embodiments of the invention.

Transient transformation can be effected by any of the direct DNA transfer methods described above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for case of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of some embodiments of the invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane. According to some embodiments of the invention, there is provided a host cell heterologously expressing an isolated polynucleotide of the invention, as described hereinabove. The host cell can be any suitable host cell include bacteria, yeast and other microorganisms that can be cultured or grown in fermentation, plant and other eukaryotic cells. For example, the host cell a bacterial cell (e.g., E. coli and B. subtilis) transformed with a heterologous nucleic acid, such as bacteriophage DNA, plasmid DNA, or cosmid DNA expression vectors containing the nucleic acid molecules described herein, or yeast (e.g., S. cerevisiae or S. pombe) transformed with recombinant yeast expression vectors containing the nucleic acid molecules described herein.

In some embodiments, the host cell is a yeast cell. In a specific embodiment, the yeast cell is a yeast cell deprived of endogenous sterol biosynthesis, such as GIL77, or a yeast line deficient in the endogenous squalene epoxidase erg1 gene such as described in Rasbery J M et al. (Jour. Biol. Chem. 2007. 282:17002-17013).

In some embodiments, the host cell produces mogrol, mogrol or mogroside precursor, or mogroside.

The methods may also employ a mixture of recombinant and non-recombinant host. If more than one host is used then the hosts may be co-cultivated, or they may be cultured separately. If the hosts are cultivated separately the intermediate products may be recovered and optionally purified and partially purified and fed to recombinant hosts using the intermediate products as substrates.

Recombinant hosts described herein can be used in methods to produce mogroside compounds. For example, if the recombinant host is a microorganism, the method can include growing the recombinant microorganism in a culture medium under conditions in which one or more of the enzymes catalyzing step(s) of the methods of the invention, e.g. synthases, hydrolases, CYP450s and/or UGTs are expressed. The recombinant microorganism may be grown in a fed batch or continuous process.

Typically, the recombinant microorganism is grown in a fermenter at a defined temperature(s) for a desired period of time. A cell lysate can be prepared from the recombinant host expressing one or more enzymes and be used to contact a substrate, such that mogroside compounds can be produced. For example, a cell lysate can be prepared from the recombinant host expressing one or more UGTs and used to contact mogrol or mogroside, such that mogroside compounds can be produced.

In some embodiments, mogroside compounds can be produced using whole cells that are fed raw materials that contain precursor molecules, e.g., mogrol. The raw materials may be fed during cell growth or after cell growth. The whole cells may be in suspension or immobilized. The whole cells may be in fermentation broth or in a reaction buffer. In some embodiments a permeabilizing agent may be required for efficient transfer of substrate into the cells.

Levels of products, substrates and intermediates can be determined by extracting samples from culture media for analysis according to published methods. Mogroside compounds can be recovered from the culture or culture medium using various techniques known in the art.

In some embodiments, there is provided a cell lysate of the host cell. Such a cell lysate can comprise both the mogroside pathway enzymes of the present invention, and the mogrol, mogrol and mogroside precursors and mogroside products of the pathways. Thus, the cell lysate can be used either for recovery of the products of the mogroside pathway (e.g. mogrol, mogroside M4, M5 and M6) or recovery of the recombinantly expressed enzymes polypeptides. Methods for extraction of active enzyme polypeptides are well known in the art.

Cell lysate of the invention can also be used for cell-free synthesis of mogrol, mogrol or mogroside precursors and mogroside, alone or in combination with other suitable substrates or enzymes.

### Recombinant Host

This document also feature recombinant hosts. As used herein, the term recombinant host is intended to refer to a host, the genome of which has been augmented by at least one incorporated DNA sequence. The incorporated DNA sequence may be a heterologous nucleic acid encoding one or more polypeptides. Such DNA sequences include but are not limited to genes that are not naturally present, DNA sequences that are not normally transcribed into RNA or translated into a protein (“expressed”), and other genes or DNA sequences which one desires to introduce into the non-recombinant host. It will be appreciated that typically the genome of a recombinant host described herein is augmented through the stable introduction of one or more recombinant genes. The recombinant gene may also be a heterologous nucleic acid encoding one or more polypeptides. Generally, the introduced DNA or heterologous nucleic acid is not originally resident in the host that is the recipient of the DNA, but it is within the scope of the invention to isolate a DNA segment from a given host, and to subsequently introduce one or more additional copies of that DNA into the same host, e.g., to enhance production of the product of a gene or alter the expression pattern of a gene. In some instances, the introduced DNA or heterologous nucleic acid will modify or even replace an endogenous gene or DNA sequence by, e.g., homologous recombination or site-directed mutagenesis.

According to a specific embodiment, the plant is of the Cucurbitaceae family. Exemplary species are provided below.

Subfamily Zanonioideae (small striate pollen grains)
Tribe Zanonieae
Subtribe Fevilleinae: Fevillea
Subtribe Zanoniinae: Alsomitra Zanonia Siolmatra Gerrardanthus Zygosicyos Xerosicyos Neoalsomitra
Subtribe Gomphogyninae: Hemsleya Gomphogyne Gynostemma
Subtribe Actinostemmatinae: Bolbostemma Actinostemma
Subtribe Sicydiinae: Sicydium Chalema Pteropepon Pseudosicydium Cyclantheropsis
Subfamily Cucurbitoideae (styles united into a single column)
Tribe Melothrieae
Subtribe Dendrosicyinae: Kedrostis Dendrosicyos Corallocarpus Ibervillea Tumamoca Halosicyos Ceratosanthes Doyerea Trochomeriopsis Seyrigia Dieterlea Cucurbitella Apodanthera Guraniopsis Melothrianthus Wilbrandia
Subtribe Guraniinae: Helmontia Psiguria Gurania
Subtribe Cucumerinae: Melancium Cucumeropsis Posadaea Melothria Muellarargia Zehneria Cucumis (including: Mukia, Dicaelospermum, Cucumella, Oreosyce, and Myrmecosicyos [4]).
Subtribe Trochomeriinae: Solena Trochomeria Dactyliandra Ctenolepsis
Tribe Schizopeponeae: Schizopepon
Tribe Joliffieae
Subtribe Thladianthinae: Indofevillea Siraitia Thladiantha Momordica
Subtribe Telfairiinae: Telfaria
Tribe Trichosantheae
Subtribe Hodgsoniinae: Hodgsonia
Subtribe Ampelosicyinae: Ampelosicyos Peponium
Subtribe Trichosanthinae: Gymnopetalum Trichosanthes Tricyclandra
Subtribe Herpetosperminae: Cephalopentandra Biswarea Herpetospermum Edgaria
Tribe Benincaseae
Subtribe Benincasinae: Cogniauxia Ruthalicia Lagenaria Benincasa Praecitrullus Citrullus Acanthosicyos Eureiandra Bambekea Nothoalsomitra Coccinia Diplocyclos Raphidiocystis Lemurosicyos Zombitsia Ecballium Bryonia
Subtribe Luffinae: Luffa
Tribe Cucurbiteae (pantoporate, spiny pollen): Cucurbita Sicana Tecunumania Calycophysum Peponopsis Anacaona Polyclathra Schizocarpum Penelopeia Cionosicyos Cayaponia Selysia Abobra
Tribe Sicyeae (trichomatous nectary, 4- to 10-colporate pollen grains)
Subtribe Cyclantherinae: Hanburia Echinopepon Marah Echinocystis Vaseyanthus Brandegea Apatzingania Cremastopus Elateriopsis Pseudocyclanthera Cyclanthera Rytidostylis
Subtribe Sicyinae: Sicyos Sicyosperma Parasicyos Microsechium Sechium Sechiopsis Pterosicyos
incertae sedis: Odosicyos
Alphabetical list of genera: Abobra Acanthosicyos Actinostemma Alsomitra Ampelosycios Anacaona Apatzingania Apodanthera Bambekea Benincasa Biswarea Bolbostemma Brandegea Bryonia Calycophysum Cayaponia Cephalopentandra Ceratosanthes Chalema Cionosicyos Citrullus Coccinia Cogniauxia Corallocarpus Cremastopus Ctenolepis Cucumella Cucumeropsis Cucumis Cucurbita Cucurbitella Cyclanthera Dactyliandra Dendrosicyos Dicaelospermum Dieterlea Diplocyclos Doyerea Ecballium Echinocystis Echinopepon Edgaria Elateriopsis Eureiandra Fevillea Gerrardanthus Gomphogyne Gurania Guraniopsis Gymnopetalum Gynostemma Halosicyos Hanburia Helmontia Hemsleya Herpetospermum Hodgsonia Ibervillea Indofevillea Kedrostis Lagenaria Lemurosicyos Luffa Marah Melancium Melothria Melothrianthus Microsechium Momordica Muellerargia Mukia Myrmecosicyos Neoalsomitra Nothoalsomitra Odosicyos Oreosyce Parasicyos Penelopeia Peponium Peponopsis Polyclathra Posadaea Praecitrullus Pseudocyclanthera Pseudosicydium Psiguria Pteropepon Pterosicyos Raphidiocystis Ruthalicia Rytidostylis Schizocarpum Schizopepon Sechiopsis Sechium Selysia Seyrigia Sicana Sicydium Sicyos Sicyosperma Siolmatra Siraitia Solena Tecunumania Telfairia Thladiantha Trichosanthes Tricyclandra Trochomeria Trochomeriopsis Tumacoca Vaseyanthus Wilbrandia Xerosicyos Zanonia Zehneria Zombitsia Zygosicyos.

Cucurbita genus refers to genus in the gourd family Cucurbitaceae native to and originally cultivated in the Andes and Mesoamerica. The Cucurbita species may be domesticated or non-domesticated.

Exemplary species include, but are not limited to:
C. argyrosperma (synonym C. mixta)-pipian, cushaw pumpkin; origin-Panama, Mexico
C. kellyana, origin-Pacific coast of western Mexico
C. palmeri, origin-Pacific coast of northwestern Mexico
C. sororia, origin-Pacific coast Mexico to Nicaragua, northeastern Mexico
C. digitata-fingerleaf gourd; origin-southwestern USA, northwestern Mexico
C. californica
C. cordata
C. cylindrata
C. palmata
C. ecuadorensis, origin-Ecuador's Pacific coast
C. ficifolia—figleaf gourd, chilacayote; origin-Mexico, Panama, northern Chile and Argentina
C. foetidissima—stinking gourd, buffalo gourd; origin-Mexico
C. scabridifolia, likely a natural hybrid of C. foetidissima and C. pedatifolia [67] [68]
C. galeottii is little known; origin-Oaxaca, Mexico
C. lundelliana, origin-Mexico, Guatemala, Belize
C. maxima—winter squash, pumpkin; origin-Argentina, Bolivia, Ecuador
C. andreana, origin-Argentina
C. moschata—butternut squash, ‘Dickinson’ pumpkin, golden cushaw; origin-Bolivia, Colombia, Ecuador, Mexico, Panama, Puerto Rico, Venezuela
C. okeechobeensis, origin-Florida
C. martinezii, origin-Mexican Gulf Coast and foothills
C. pedatifolia, origin-Querétaro, Mexico
C. moorei
C. pepo—field pumpkin, summer squash, zucchini, vegetable marrow, courgette, acorn squash; origin-Mexico, USA
C. fraterna, origin-Tamaulipas and Nuevo León, Mexico
C. texana, origin-Texas, USA
C. radicans-calabacilla, calabaza de coyote; origin-Central Mexico C. gracilior

The polypeptides, polynucleotides, cells and methods of the present invention can be used to produce mogroside VI. Thus, according to some embodiments, there is provided a composition enriched in mogroside VI to a total concentration of mogroside VI of at least 10% (wt/wt).

In some embodiments, and especially in populations of recombinant cells producing mogroside, mogrosides MII and MV or MVI may be found together in significant amounts. Thus, according to one embodiment, there is provided a composition comprising mogroside VI (M6) and mogroside II (M2), and or a composition comprising mogroside V (M5), VI (M6) and mogroside II (M2).

In some embodiments, especially where the composition comprising the mogroside is produced in recombinant cells heterologously expressing one or more of the mogrol biosynthesis pathway enzymes of the invention, the composition comprises mogroside M4, and/or M5 and or M6, and DNA comprising at least one DNA sequence encoding the one or more mogrol biosynthesis pathway enzymes, the DNA sequence lacking at least one intron. In some embodiments, the sequence is 10%, 20%, 30%, 40%, 50%, 60% or more of the complete coding sequence of the mogrol biosynthesis pathway polypeptide. In some cases the at least one DNA sequence comprising the coding sequence comprises a coding sequence optimized for expression in a recombinant host, and differing in the nucleic acid sequence from the native (e.g. S. grosvenorii) sequence by at least 5%, at least 10%, at least 15%, at least 20% or more.

In some embodiments, wherein an enhanced sweetness is desired, a concentration of the mogroside VI or mogroside V is sufficient to cause an enhancement in flavor, and can be used as a sweetener. Such a composition can comprise a concentration of the mogroside VI of at least 0.2 ppm (e.g., 0.2-300) ppm or more.

In some embodiments, the composition of the invention is a consumable composition.

Consumables include all food products, including but not limited to, cereal products, rice products, tapioca products, sago products, baker's products, biscuit products, pastry products, bread products, confectionery products, desert products, gums, chewing gums, chocolates, ices, honey products, treacle products, yeast products, baking-powder, salt and spice products, savory products, mustard products, vinegar products, sauces (condiments), tobacco products, cigars, cigarettes, processed foods, cooked fruits and vegetable products, meat and meat products, jellies, jams, fruit sauces, egg products, milk and dairy products, yoghurts, cheese products, butter and butter substitute products, milk substitute products, soy products, edible oils and fat products, medicaments, beverages, carbonated beverages, alcoholic drinks, beers, soft drinks, mineral and aerated waters and other non-alcoholic drinks, fruit drinks, fruit juices, coffee, artificial coffee, tea, cocoa, including forms requiring reconstitution, food extracts, plant extracts, meat extracts, condiments, sweeteners, nutraceuticals, gelatins, pharmaceutical and non-pharmaceutical gums, tablets, lozenges, drops, emulsions, elixirs, syrups and other preparations for making beverages, and combinations thereof.

Mogroside compositions of the invention can be used in various consumables including but not limited to water-based consumables, solid dry consumables and dairy products, dairy-derived products and dairy-alternative products. In some embodiments the composition is a foodstuff.

Water-based consumables include but are not limited to beverage, water, aqueous drink, enhanced/slightly sweetened water drink, mineral water, carbonated beverage, non-carbonated beverage, carbonated water, still water, soft drink, non-alcoholic drink, alcoholic drink, beer, wine, liquor, fruit drink, juice, fruit juice, vegetable juice, broth drink, coffee, tea, black tea, green tea, oolong tea, herbal tea, cacao (water-based), tea-based drink, coffee-based drink, cacao-based drink, syrup, frozen fruit, frozen fruit juice, water-based ice, fruit ice, sorbet, dressing, salad dressing, sauce, soup, and beverage botanical materials (whole or ground), or instant powder for reconstitution (coffee beans, ground coffee, instant coffee, cacao beans, cacao powder, instant cacao, tea leaves, instant tea powder). In some embodiments, the composition can be a beverage such as Coca-Cola® and the like.

Solid dry consumables include but are not limited to cereals, baked food products, biscuits, bread, breakfast cereal, cereal bar, energy bars/nutritional bars, granola, cakes, cookies, crackers, donuts, muffins, pastries, confectioneries, chewing gum, chocolate, fondant, hard candy, marshmallow, pressed tablets, snack foods, and botanical materials (whole or ground), and instant powders for reconstitution as mentioned above.

For water-based or solid dry consumables a useful concentration may be from 0.2 ppm (e.g., 0.2-300) ppm or more.

In certain products a higher sweetener concentration is usually necessary to reach similar sweetness intensity, for example in dairy products, dairy-derived products and dairy-alternative products. Dairy-derived food products contain milk or milk protein. Dairy-alternative products contain (instead of dairy protein derived from the milk of mammals) protein from botanical sources (soy, rice, and other protein-rich plant materials). Dairy products, dairy-derived products and dairy-alternative products include but are not limited to milk, fluid milk, cultured milk product, cultured and noncultured dairy-based drinks, cultured milk product cultured with lactobacillus, yoghurt, yoghurt-based beverage, smoothy, lassi, milk shake, acidified milk, acidified milk beverage, butter milk, kefir, milk-based beverage, milk/juice blend, fermented milk beverage, icecream, dessert, sour cream, dip, salad dressings, cottage cheese, frozen yoghurt, soy milk, rice milk, soy drink, rice milk drink.

Milk includes, but is not limited to, whole milk, skim milk, condensed milk, evaporated milk, reduced fat milk, low fat milk, nonfat milk, and milk solids (which may be fat or nonfat).

For dairy products, dairy-derived products and dairy-alternative products, a useful concentration will be from about 0.3 to 500 ppm or higher, and may be up to 550 ppm, 600 ppm, 650 ppm, 700 ppm, or 750 ppm.

The composition of the invention can also include one or more additional flavor ingredients, such as additional sweeteners. A non-limiting list of suitable flavor ingredients useful with the composition of the invention includes sucrose, fructose, glucose, high fructose corn syrup, xylose, arabinose, rhamnose, erythritol, xylitol, mannitol, sorbitol, inositol, AceK, aspartame, neotame, sucralose, saccharine, naringin dihydrochalcone (NarDHC), neohesperidin dihydrochalcone (NDHC), rubusoside, rebaudioside A, stevioside, stevia and trilobtain.

Sweeteners commonly used in consumables include:
Acesulfame K—Artificial Sweetener (E950)
Agave Syrup—Modified Sugar
Alitame—Artificial Sweetener (E956)
Aspartame—Artificial Sweetener (E951)
Aspartame-Acesulfame Salt—Artificial Sweetener (E962)
Barley Malt Syrup—Modified Sugar
Birch Syrup—Sugar Extract
Blackstrap Molasses—Sugar Extract
Brazzein—Natural Sweetener
Brown Rice Syrup—Modified Sugar
Cane Juice—Sugar Extract
Caramel—Modified sugar
Coconut Palm Sugar—Sugar Extract
Corn Sugar (HFCS)—Modified sugar
Corn Sweetener (HFCS)—Modified sugar
Corn Syrup (HFCS)—Modified sugar
Curculin—Natural Sweetener
Cyclamate—Artificial Sweetener (E952)
Dextrose—Sugar
Erythritol—Sugar Alcohol (E968)
Fructose Glucose Syrup (HFCS)—Modified sugar
Fructose—Sugar
Galactose—Sugar
Glucitol (Sorbitol)—Sugar Alcohol (E420)
Glucose—Sugar
Glucose Fructose Syrup (HFCS)—Modified sugar
Glycerol (Glycerin)—Sugar Alcohol (E422)
Glycyrrhizin—Natural Sweetener (E958)
Golden Syrup—Modified sugar
High Fructose Corn Syrup (HFCS)—Modified Sugar
HFCS-42—Modified Sugar
HFCS-55—Modified Sugar
HFCS-90—Modified Sugar
Honey—Natural Sugar
HSH—Sugar Alcohol
Hydrogenated Starch Hydrolysate (HSH)—Sugar Alcohol
Isoglucose (HFCS)—Modified sugar
Inulin—Sugar Fiber
Inverted Sugar—Modified sugar
Isomalt—Sugar Alcohol (E953)
Lactitol—Sugar Alcohol (E966)
Lactose—Sugar
Levulose (Fructose)—Sugar
Luo Han Guo—Natural Sweetener
Maltitol—Sugar Alcohol (E965)
Maltodextrin—Sugar
Maltose—Sugar
Mannitol—Sugar Alcohol (E421)
Maple Syrup—Sugar Extract
Miraculin—Natural Sweetener
Molasses—Sugar Extract
Monellin—Natural Sweetener
Monk Fruit (Luo Han Guo)—Natural Sweetener
Neohesperidin DC—Artificial Sweetener (E959)
Neotame—Artificial Sweetener (E961)
Oligofructose—Sugar Fiber
Palm Sugar—Sugar Extract
Pentadin—Natural Sweetener
Rapadura—Sugar Extract
Refiners Syrup—Modified Sugar
Saccharin,—Artificial Sweetener (E954)
Saccharose (Sucrose)—Sugar
Sorbitol—Sugar Alcohol (E420)
Sorghum Syrup—Sugar Extract
Stevia—Natural Sweetener
Stevioside—Natural Sweetener (E960)
Sucralose—Artificial Sweetener (E955)
Sucrose—Sugar
Tagatose—Modified Sugar
Thaumatin—Natural Sweetener (E957)
Trehalose—Sugar
Xylitol—Sugar Alcohol (E967)
Yacon Syrup—Natural Sweeten

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization-A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Experimental Procedures

### Gene Screen

In order to identify candidate Siraitia genes that may be involved in mogroside biosynthesis the present inventors have performed a detailed transcriptome analysis of 6 stages of developing Siraitia fruit. The fruit stages were 15, 34, 55, 77, 93 and 103 days after fruit set, which was accomplished by spraying the anthesis female flowers with a commercial fruit set hormone (20 ppm NAA naphthaleneacetic acid, commercial formulation Alphatop, Perelman Co. Tel Aviv, Israel) treatment commonly used for the production of parthenocarpic squash fruit. Developing fruits were sampled, stored at −80 C and used for further analyses. RNA from powdered fruit samples was extracted and transcripts were prepared using the Tru Seq® RNA Sample Preparation Kit v2 (Illumina San Diego, California, USA) according to manufacturer's directions. RNA-seq libraries were analyzed using Illumina HiSeq2500 technology at the University of Illinois Genome Research Center and reads were assembled into transcript contigs using standard de novo assembly packages. Transcripts were annotated against public genome databases including NCBI non-redundant proteins (nr), and cucurbit genomics databases such as the melon genome (https://melonomics(dot)net/) and cucumber genome (www(dot)cugi(dot)org). Transcripts annotated as candidate genes for the various enzymes involved in the metabolism of mogrosides (squalene epoxidase, cucurbitadienol synthase, epoxide hydrolase, cytochrome P450 and UDPglucose glucosyltransferase) were selected for heterologous expression and functional analysis. The same fruit samples were analyzed for mogroside content in order to determine the stages of successive additions of glucosyl groups.

### Tissue Sampling for Metabolic Profiling

Tissue preparation—For HPLC, fresh or frozen (−80° C.) fruit tissue was ground in liquid nitrogen using IKA A11 grinder. Then 600 μl of methanol: water (1:1) was added to 200 mg fine ground powder and the resulting mixture was vortexed for 30 seconds, sonicated for 15 min and vortexed again for 30 seconds. The sample was clarified of debris by centrifugation (20,000×g) and by filtration using Axiva syringe filters (PTFE, 0.2 μm).

HPLC-DAD—The analysis was carried out on an Agilent 1200 HPLC system with an Agilent 1200 Diode Array Detector (DAD). The analytical column: Zorbax Stable Bond-C18 column (4.6×150.0 mm, 5.0 μm, Agilent Technologies, USA). The mobile phase contained A, H2O with 0.1% formic acid; B, 100% HPLC grade acetonitrile. The column was equilibrated with 80% A, and then the sample was injected, reaching 90% B gradient after 10 min. The mobile phase flow was 1.5 ml min-1. Each substance was identified by co-migration with commercial standards and by matching the spectrum of each nucleoside peak against that of a standard.

HPLC-MS—The analysis was carried out on an Agilent 1290 Infinity series liquid chromatograph coupled with an Agilent 1290 Infinity DAD and Agilent 6224 Accurate Mass Time of Flight (TOF) mass spectrometer (MS). The analytical column was: Zorbax Extend-C18 Rapid Resolution HT column (2.1×50.0 mm, 1.8 μm, Agilent Technologies, Waldbronn, Germany) Mass spectrometry was performed using an Agilent 6224 Accurate Mass TOF LC/MS System equipped with a dual-sprayer orthogonal ESI source, with one sprayer for analytical flow and one for the reference compound (Agilent Technologies, Santa Clara, USA). The mobile phase contained A, H2O with 0.1% formic acid; B, 100% HPLC grade acetonitrile. The column was equilibrated with 80% A, and then the sample was injected, reaching 90% B gradient after 10 min. The mobile phase flow was 0.4 ml min-1. Each substance was identified by co-migration with commercial standards and by matching the mass spectrum of putative peak against that of a standard. The chromatogram was initially analyzed by MassHunter Qualitative Analysis software v.B.05.00 (Agilent) and further analyzed by MassHunter Mass Profiler software v.B.05.00 (Agilent).

### UGT Expression and Functional Analysis

For UGT expression, which was carried out in an E. coli expression system, the resulting plasmid was transformed to E. coli Arctic Express (Agilent). For expression of the UGT enzyme, a fresh overnight culture was diluted 1:100 in 25 ml LB medium with 50 μg/ml kanamycin and gentamicin, and incubated at 37° C. and 250 rpm until an A600 of 0.4 was reached. Subsequently, IPTG was added to a concentration of 0.5 mM, and the incubation was continued overnight at 18° C. and 250 rpm. The next day, cells were harvested by centrifugation, and the pellet resuspended in 2 ml of 50 mM Tris HCl PH=7.0 and 5 mM β-mercaptoethanol. After breaking the cells by sonication, insoluble material was removed by centrifugation, and the soluble fraction was used for characterization of the enzyme. Protein was stored at −20° C. until further analysis.

### UGT Assays:

Substrates (mogrosides) were dissolved to 1 mM in 50% DMSO. Enzyme assays were carried out in 50 mM Tris HCl pH=7.0 and 5 mM β-mercaptoethanol using 8 mM UDP-xylose and 0.1 mM substrate and 25 ul of enzyme crude extract (reaction in an end volume of 100 μl). After overnight incubation at 30° C., reactions were stopped by addition of 300 μl methanol and 0.1% formic acid. Samples were prepared by brief vortexing. Then the extracts were centrifuged for 15 min at 13,000 rpm and analyzed on LC-MS. The product was compared to a control incubation which contained an enzyme preparation of an E. coli harboring an empty pET28a.

### Example 1

### Temporal Pattern of Mogroside Accumulation

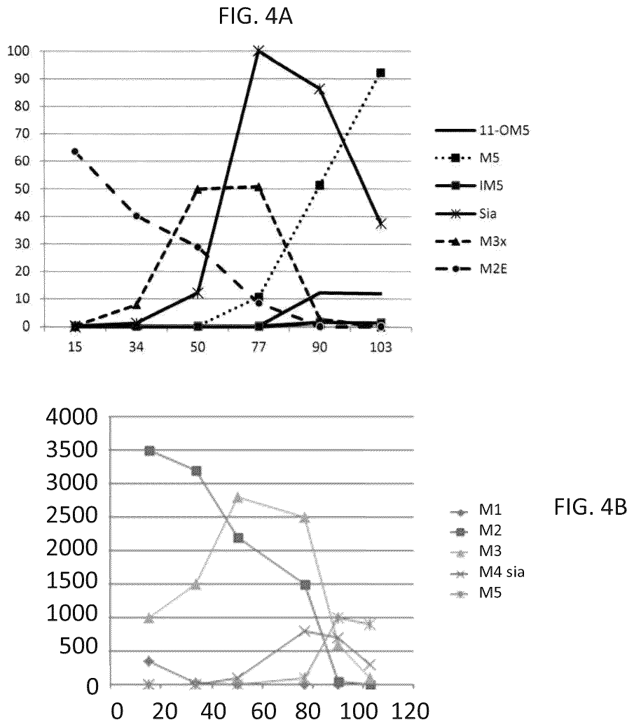

Mogroside accumulation during development of the Siraitia fruit is shown in FIGS. 4A and 4B. Targeted metabolic profiling of Siraitia mogrosides during fruit ripening was carried out on methanolic extracts of the frozen powders and analyzed by HPLC with photodiode array and mass spec detection. Results reveal their unique temporal distribution. Mogrosides were limited to the developing fruit and were not observed in the root, stem or leaf tissue.

Already in the youngest stage of immature fruit analyzed, at 15 DAA (Days After Anthesis), the majority of the mogrols were present in the di-glucosylated form in which the C-3 and C-24 mogrol carbons are each mono-glucosylated. Non-glucosylated, mono-glucosylated or alternative M2 compounds, in which the second glucosyl moiety was present as a branched glucose on one of the primary glucose moieties, were not observed, indicating that the initial metabolic steps of mogroside glucosylations are limited to the two primary glucosylations and that these occur early in fruit development.

The total mogroside levels in the developing fruitlets remained similar throughout development and there was no indication of a net accumulation of mogrosides with development. These results indicate a strong temporal division of mogroside metabolism and that the early steps of mogrol synthesis and the initial primary glucosylations are limited to early fruit development, preparing the reservoir of mogrosides for subsequent glucosylations.

Following the synthesis of M2 there is an additional branched 1-6 glycosylation at the C24 position leading to the accumulation of M3X. During the later stages (77 and 90 DAA) a number of M4 compounds appeared, primarily siaminoside which was confirmed by NMR as the third branched glucosylation at the C24 position. Alternative tetra-glucosylated mogrosides, such as M4A, were also present, but in low amounts. M5, with a second glucosylation at the C3 position, began to accumulate at the expense of the M4 compounds at 77 DAA and increased sharply during the final stages of ripening. In the ripe 103 DAA fruit M5, along with small traces of IM5, comprised the majority of fruit mogroside components. (FIG. 4B).

Thus, at the youngest stage analyzed there was already the full complement of mogroside metabolites up to the diglucosylated mogrol, M2. Expression of candidate genes for the early stages of mogroside synthesis, including specifically squalene epoxidase, epoxide hydrolase, cucurbitadienol synthase, cyp450 and the primary glucosylation UGTs, was then undertaken

### Gene Cloning and Synthesis

In general, synthetic genes were ordered from Gen9Bio (Cambridge MA, USA) and subcloned into pET28a vector using NheI and NotI restriction enzymes, and the inserts were verified by sequencing.

The following examples indicate the process used to identify the genes responsible for the pathway.

### Example 2

### Identification of Siraitia Cucurbitadienol Synthase (SgCDS) as the Enzyme which Cyclicizes Both 2,3-Monoepoxysqualene and 2,3;22,23-Diepoxysqualene, Leading to, Respectively, Cucurbitadienol and 24,25-Epoxycucurbitadienol

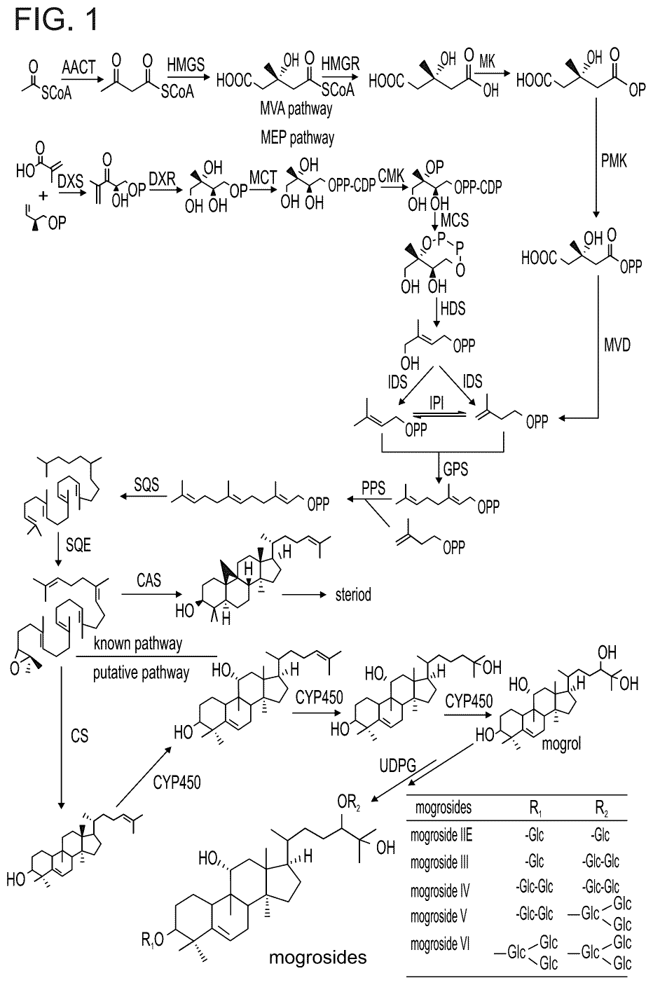

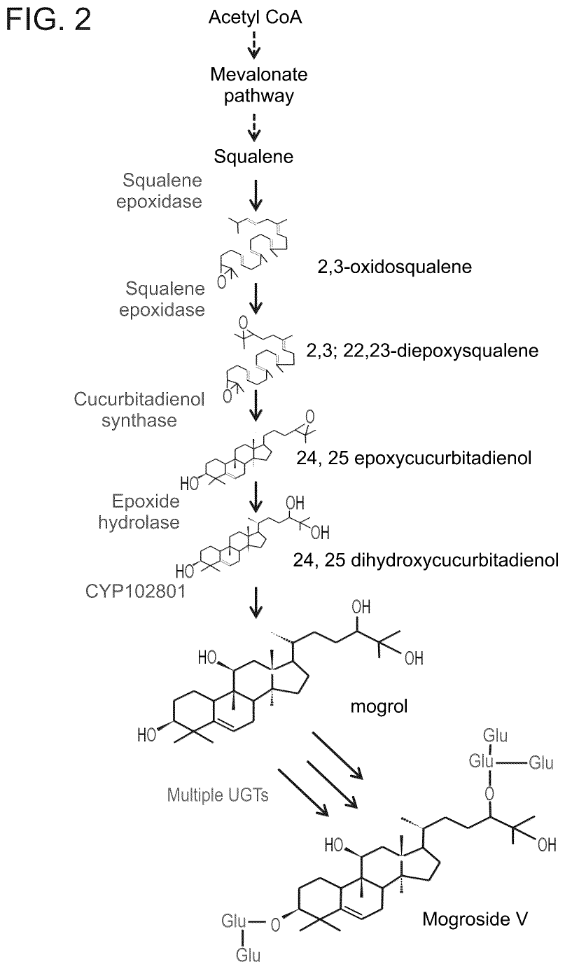

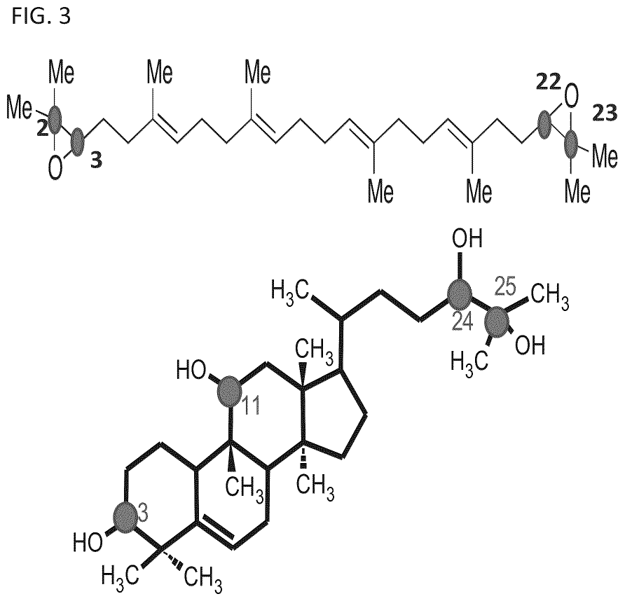

The preferred substrate for the synthesis of the novel trans-C24,C25-dihydroxycucurbitadienol is 2,3;22,23-diepoxysqualene which is symmetrically epoxidated at both ends of the squalene molecule at the squalene numbered positions of C2,3 and C22,23 (FIG. 3). 2,3;22,23-diepoxysqualene is synthesized by the enzyme squalene epoxidase (SQE) which is ubiquitous in squalene metabolizing organisms, including the yeast strain GIL77. The yeast strain GIL77 is one of the strains in which the yeast gene erg7 encoding lanosterol synthase is mutated and non-functional, thereby making available the 2,3-epoxysqualene precursor to the cucrbitadienol synthase cyclization reaction and allowing for the synthesis of cucurbitadienol. This has previously been shown for the Cucurbita species CDS gene (referred to as CPQ in Shibuya M et al 2004. Tetrahedron 60:6995-7003). While it is known that cucurbitadienol synthase can cyclicise 2,3epoxysqualene to cucurbitadienol (FIG. 1), it was not known whether it can cyclicize the 2,3;22,23-diepoxysqualene to the 24,25-diepoxycucurbitadienol, which is the key intermediate in the proposed mogroside synthesis pathway of Siraitia (FIG. 2).

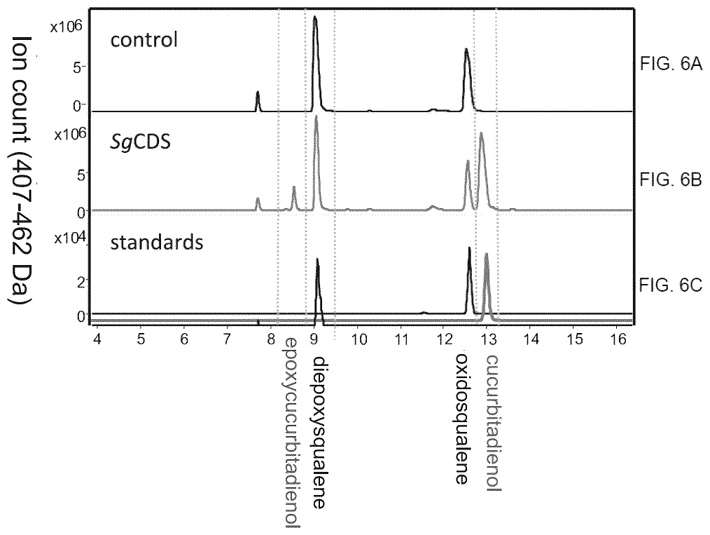

Surprisingly, it was found that the Siraitia gene coding for cucurbitadienol synthase SgCDS carries out the cyclization of both 2,3-epoxysqualene, leading to cucurbitadienol, and of 2,3;22,23-diepoxysqualene, leading to the critical substrate for the mogrol synthetic pathway, 24,25-epoxycucurbitadienol. The SgCDS gene (sequence gb/AEM42982) was heterologously expressed in the GIL77 yeast strain as described in Davidovich-Rikanati et al. (Yeast. 2015. 32 (1): 103-114). In brief, transformed yeast were cultured and the GAL1 promoter was induced by replacing the glucose carbon source by galactose. Following 2 days of induction the yeast were disrupted in presence of 20% KOH: 50% EtOH sterols were extracted with hexane. The resulting cell extracts were subjected to LC-TOF-MS analysis using APCI interphase and the chromatograms are presented in FIG. 6B. The GIL77 control culture (FIG. 6A) produced both 2,3-epoxysqualene (R.T. 12.6) and 2,3;22,23-diepoxysqualene (R.T. 9.0), due to endogenous yeast erg Isqualene epoxidase enzyme activity. Expression of SgCDS (FIG. 6B) led to the accumulation of not only cucurbitadienol but also to the accumulation of the 24,25-epoxycucurbitadienol, the appropriate substrate for the following reaction of epoxide hydrolase.

Squalene epoxidase enzymes have previously been reported to carry out both mono and diepoxidation of squalene. This has been shown to function in both animal systems (i.e., the synthesis of 24,25-epoxycholesterol in cholesterol metabolism, Nelson J A et al., Jour. Biol. Chem. 1981. 256, 1067-1068; Bai M, et al., Bioch. Biophys. Res. Comm. 1992. 185:323-329) and plant systems (i.e., Rasbery J M et al., Jour. Biol. Chem. 2007. 282:17002-17013).

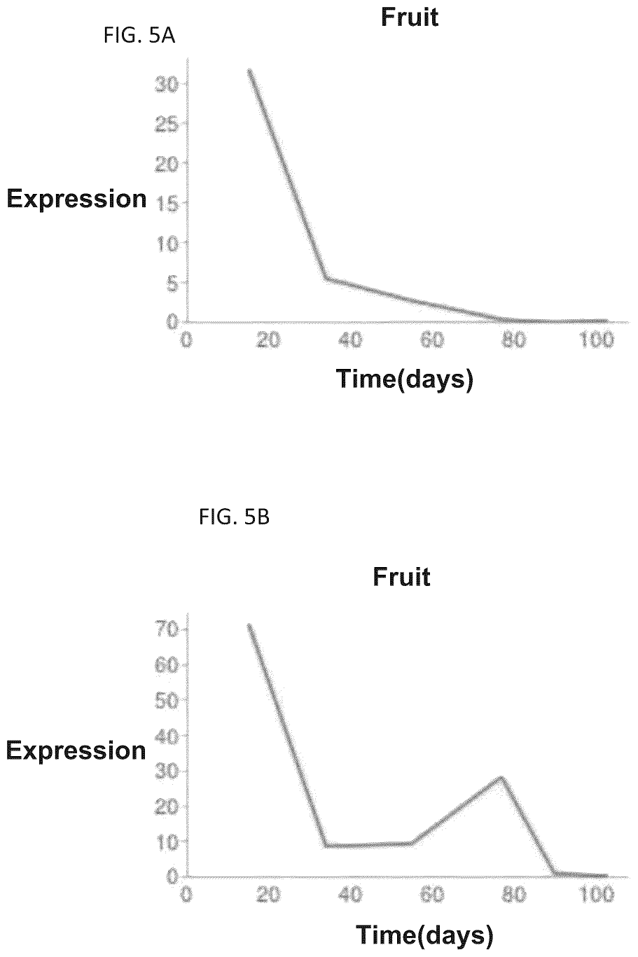

In order to identify candidate Siraitia squalene epoxidase genes that may be involved in mogrol biosynthesis a detailed transcriptome analysis of 6 stages of developing Siraitia fruit was performed. The fruit stages were 15, 34, 55, 77, 93 and 103 days after fruit set, and used for the production of transcriptome and mogroside metabolome that are described above. Data mining of Siraitia transcriptome led to the selection of 2 candidate squalene epoxidase enzymes (contigs 16760 and 18561) with high and early expression during fruiting (FIGS. 5A and 5B). These squalene epoxidase genes can be cloned and expressed in yeast, such as the line deprived of endogenic sterol biosynthesis (Gil77) as above) or a yeast line deficient in the endogenous squalene epoxidase erg1 gene such as described in Rasbery J M et al. (Jour. Biol. Chem. 2007. 282:17002-17013) and the products assayed for production of the mogrol precursor, 2,3;24,25-diepoxysqualene which can then be cyclized to 24,25-epoxycucurbitadienol and proceed through the mogrol biosynthetic pathway.

### Example 3

### Identification of S. grosvenorii Epoxy Hydratase Enzymes Catalyzing the Hydration of 24,25-Epoxycucurbitadienol in Mogrol Biosynthesis

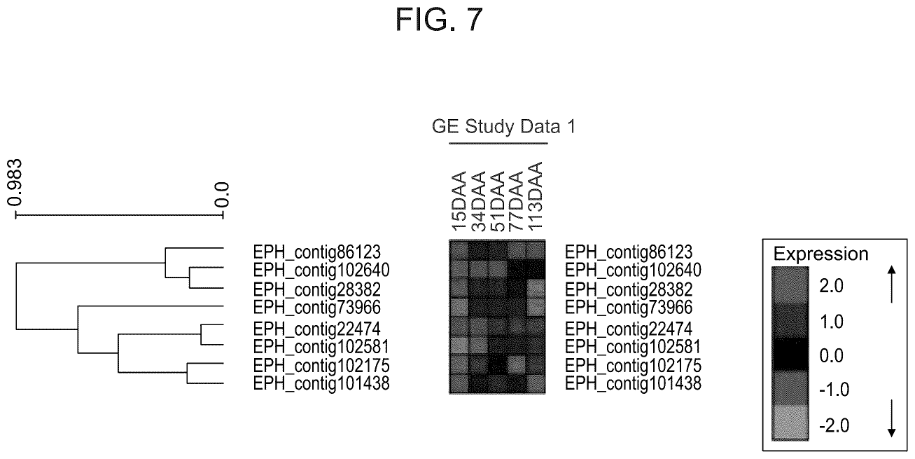

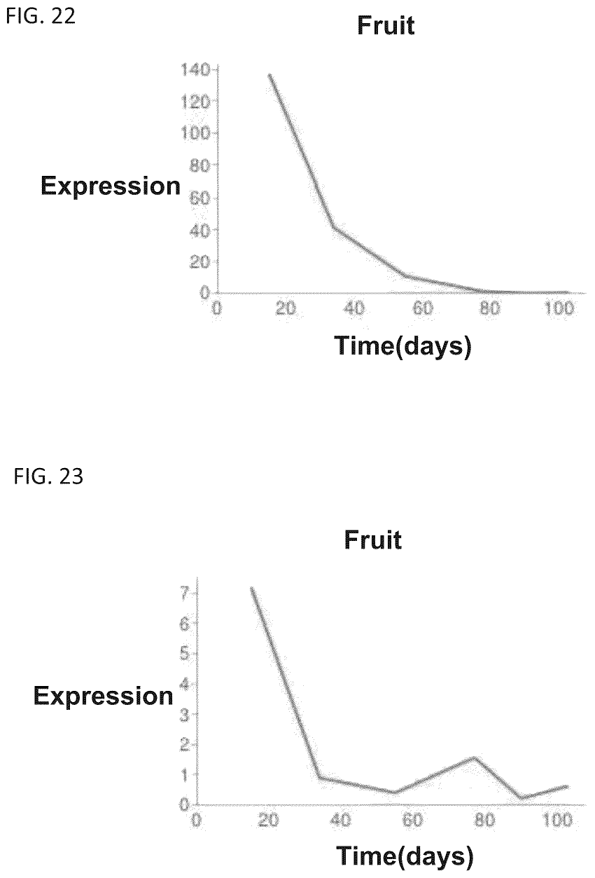

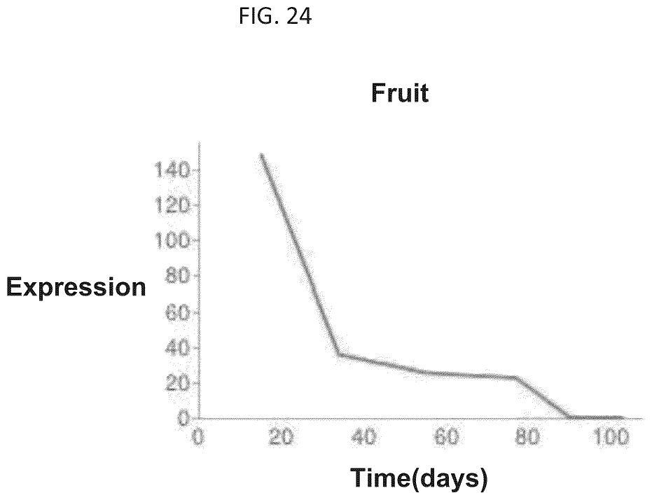

In order to identify candidate Siraitia epoxy hydratase genes that may be involved in mogrol biosynthesis a detailed transcriptome analysis of 6 stages of developing Siraitia fruit was performed. The fruit stages were 15, 34, 55, 77, 93 and 103 days after fruit set, and used for the productions of transcriptome and mogroside metabolome that are described above. Data mining of Siraitia transcriptome led to the identification and isolation of 4 candidate epoxy hydratase enzymes (contigs 73966, 86123, 102640 and 28382) with high levels of expression early in fruit development (FIGS. 7 and 21-24).

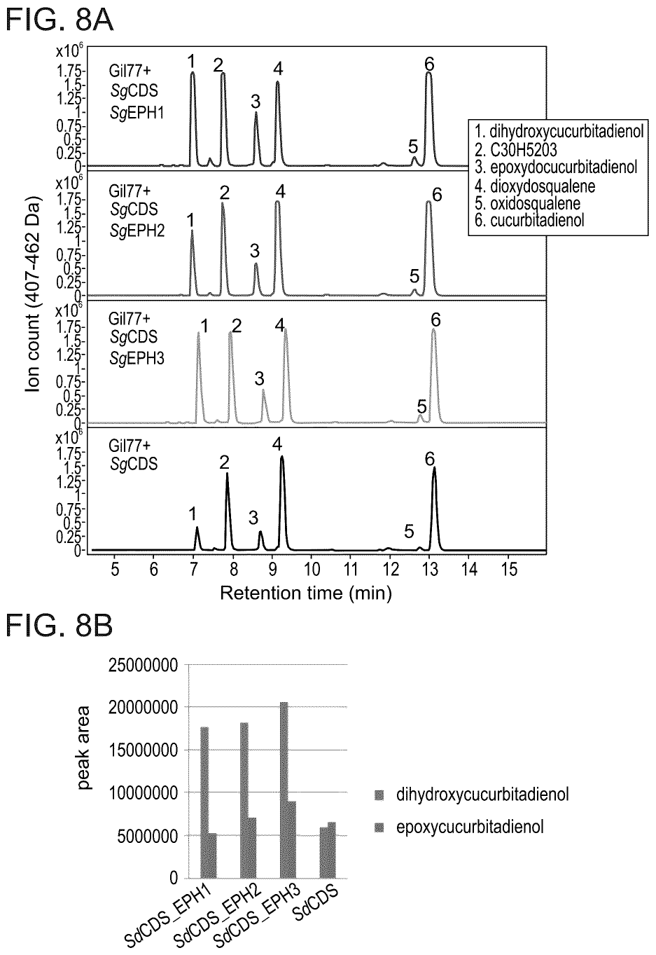

The epoxy hydratase genes were expressed in GIL77 yeast, and the products assayed for production of 24,25-dihydroxycucurbitadienol from 24,25-epoxycucurbitadienol, the product of the previously described SgCDS reaction. FIGS. 8A and 8B show the effect of heterologous expression the three EPH candidate genes (coding sequences EPH1-SEQ ID NO: 17, EPH2-SEQ ID NO: 19 and EPH3-SEQ ID NO: 21) in the GIL77 yeast strain harboring the SgCDS gene. Cmp1(peak) represents the 24,25-dihydroxycucurbitadienol product and Cmp3 (peak) represents the 24,25-epoxycucurbitadienol substrate. The results show that the expression of the S. grosvenorii SgEPH genes led to a large increase in the amount of the 24,25-dihydroxycucurbitadienol product (quantitative display—by area under peak—is shown in FIG. 8B). Due to endogenous yeast epoxide hydrolase activity, the control strain without the SgEPH) genes also accumulates 24,25-dihydroxycucurbitadienol, but to a much lower level (Gil77+SgCDS).

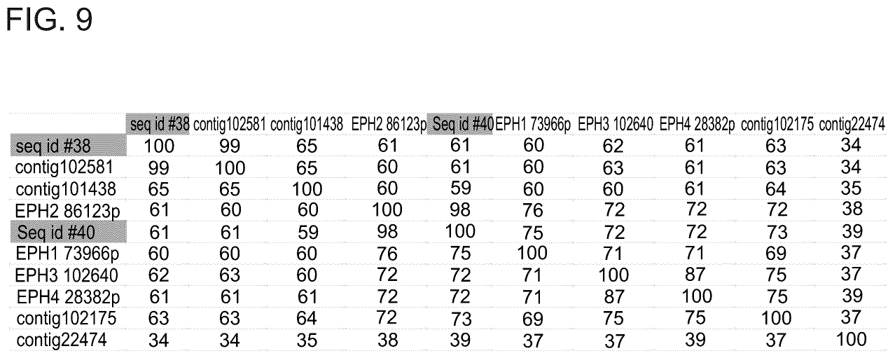

FIG. 9 shows the amino acid sequence identity matrix between the eight EPH genes of Siraitia which were identified in our transcriptomic and genomic analyses and the two EPH sequences reported by Tang et al., (2011) and subsequently used to produce tetrahydroxy squalene in WO2014086842 (identified as Seq Id Nos. 38 and 40 of WO2014086842).

Accordingly, the results of this example show that the genes identified as EPH genes in the Siraitia transcriptome are capable of carrying out the novel trans-24,25 dihydroxylation step following the CDS catalyzed cyclization of squalene diepoxide.

### Example 4

### Identification of Cucurbitadienol 11-Hydrolase

In order to identify candidate Siraitia cytochrome p450 genes that may be involved in mogrol biosynthesis a detailed transcriptome analysis of 6 stages of developing Siraitia fruit was performed. The fruit stages were 15, 34, 55, 77, 93 and 103 days after fruit set, and used for the productions of transcriptome and mogroside metabolome that are described above.

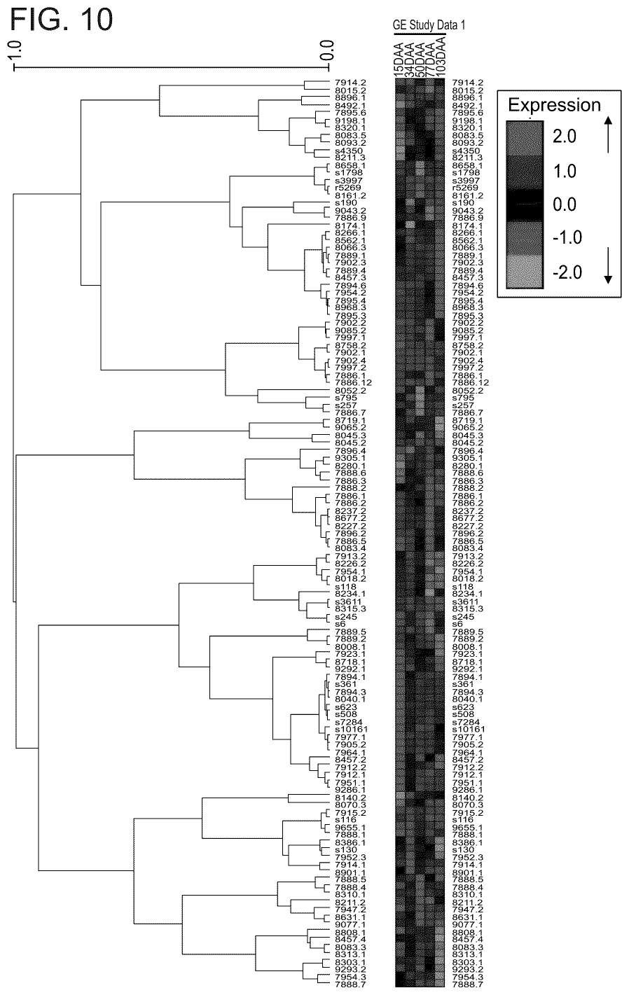

The Siraitia transcriptome indicated that the cyp450 family comprises over 100 members. Data mining of the Siraitia transcriptome based on homology analysis and expression patterns resulted in about 50 cytochrome CYP450 homologs that were expressed in developing fruits (FIG. 10) and therefore chosen for functional expression to test their activity in presence of cucurbitadienol.

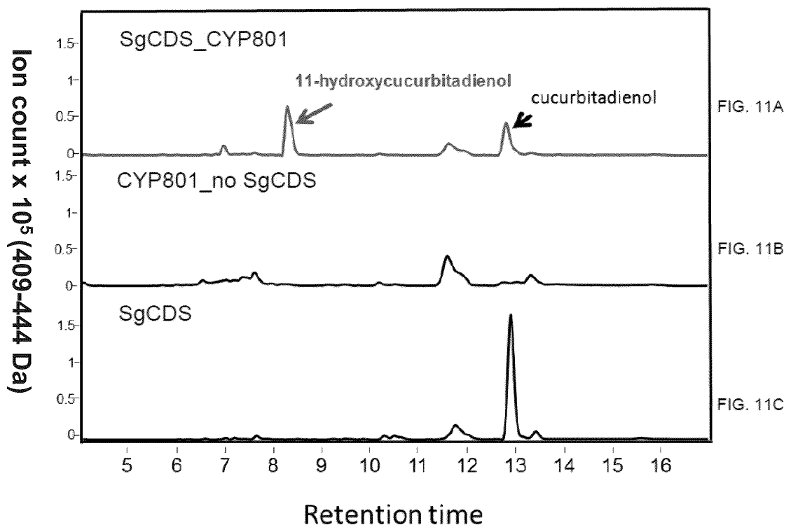

To test the possible involvement of the candidate p450s in mogrol biosynthesis and test their functionality, nucleotide sequences of all candidates were synthesized (Gen9Bio, Cambridge, MA, USA) according to their deduced full length open reading frames, and cloned in a yeast expression vector system. The candidate p450 were cloned into the dual expression pESC-URA vector system (Agilent Technologies) possessing two multiple cloning sites (MCS) for gene expression of two genes under the galactose inducible GAL1 and GAL10 promoters. Each candidate CYP was introduced into MCS 2 while the SgCDS was cloned in MCS1 and produced cucurbitadienol when induced. The resulting plasmids were transferred to S. cerevisiae strain BY4743_YHR072 (MATa/α his3Δ1/his3Δ1 leu2Δ0/leu2Δ0 LYS2/lys2Δ0 met15Δ0/MET15 ura3Δ0/ura3Δ0 kanMax::erg7/ERG7) originating from the yeast deletion project collection (Brachmann C B et al Yeast 14 (2): 115-32) that is heterozygous for lanosterol synthase, Erg7 (Corey E J et al. Proc Natl Acad Sci USA 91:2211-2215.). To aid p450 activity by supplying a proton source, all yeasts were transformed with the pESC-HIS vector harboring the Arabidopsis thaliana NADPH cytochrome p450 reductase (ArCPR1). Transformed yeast were cultured and the GAL1 promoter was induced by replacing the glucose carbon source by galactose and extracted as described in Example 2. The resulting cell extracts were subjected to LC-TOF-MS analysis using APCI interphase. The extracted ion chromatograms of the transformed yeast extracts are shown in FIG. 11A-11C. The heterologous expression of contig102801 next to SgCDS and AtCRP1 resulted in two major eluting compounds at 8.25 and 8.3 min with the designated molecular formula of C30H5002 and C30H4802 according to their exact mass of 443.3883 and 441.3727 respectively (FIG. 11A). The main product eluting at 8.3 min was further isolated for its chemical analysis by NMR to identify the OH position that was found to be on C11 of cucurbitadienol. The expression of the same contig without SgCDS resulted in no new compounds (FIG. 11B) indicating that the encoded enzyme acts on cucurbitadienol and not on lanosterol that is endogenically produced by yeast.

### Example 5

### Preparation of Mogroside Precursor Substrates for UGT Assays

Candidate UGT gene sequences were synthesized (BioGen9, Cambridge, MA, USA) and genes were individually expressed in E. coli cells. In parallel, substrates for each of the glucosylation reactions were purified, including mogrol, M1-E1 M2-A1, M2Δ, M3, M3×, siamenoside, M4, M5 (depicted in FIG. 12). These substrates were either purified from commercial mogroside powder (for compounds of M4 and above, described in (VSP Chaturvedula, I Prakash, Journal of Carbohydrate Chemistry, 2011 30:16-26 DOI: 10.1080/07328303.2011.583511 and additional mogrosides described in Sai Prakash Chaturvedula V. and Prakash I., IOSR Journal of Pharmacy. 2012 2 (4): 2250-3013) or by chemical and enzymatic hydrolysis of purified M5 and subsequent purification by HPLC.

### Primary Glucosylations

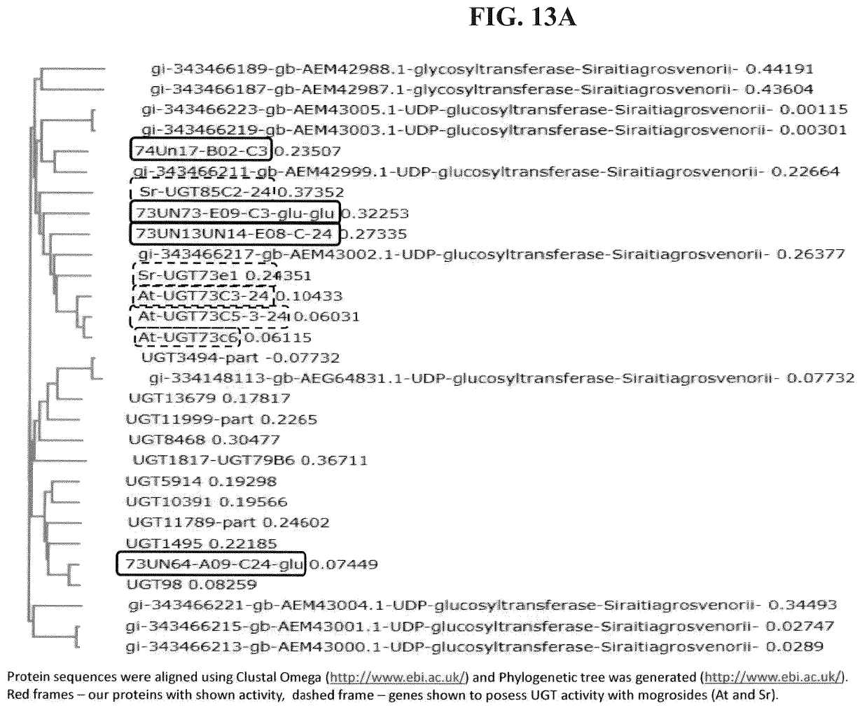

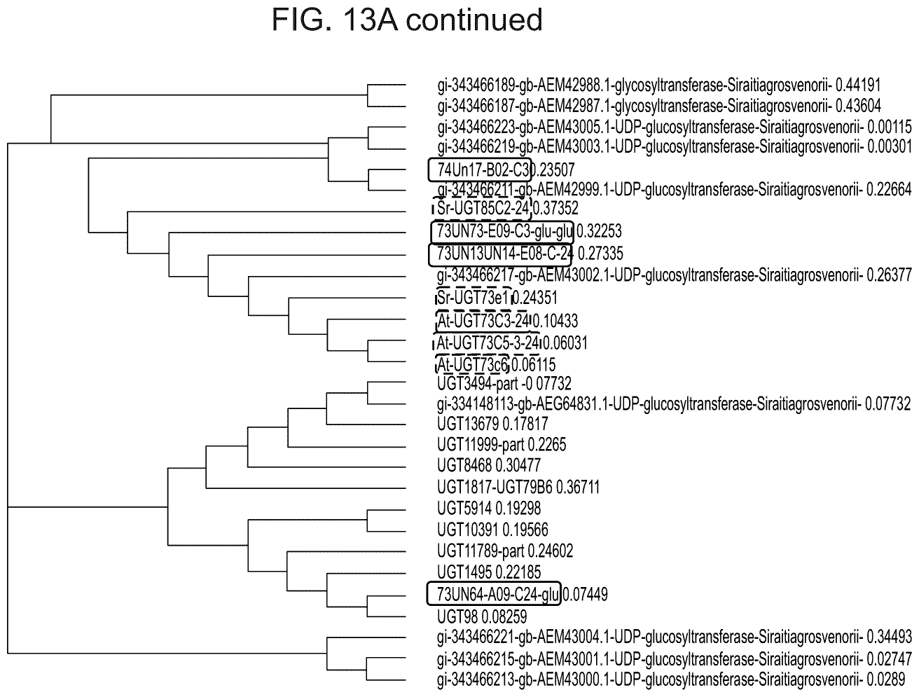

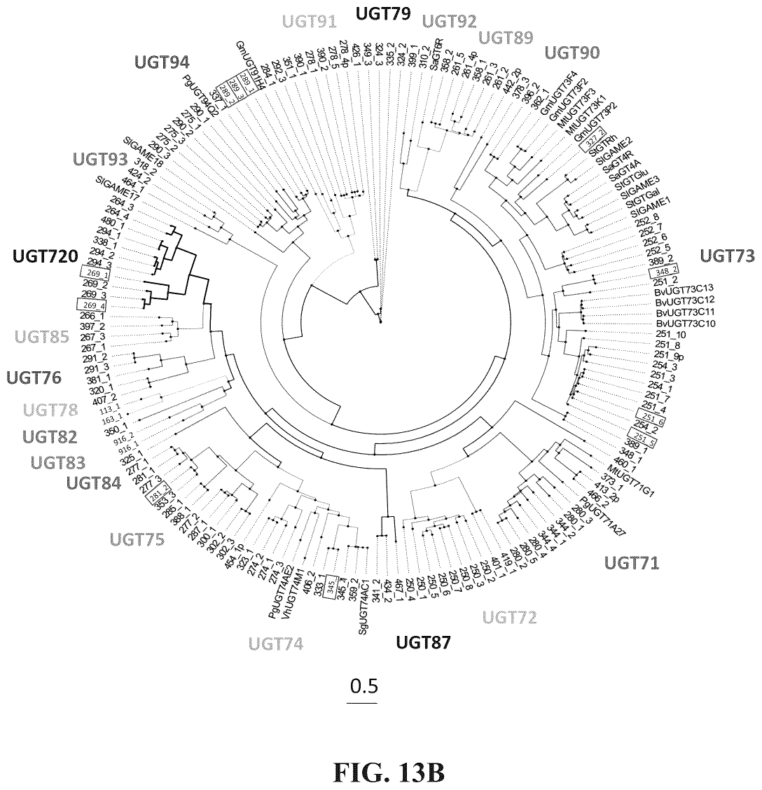

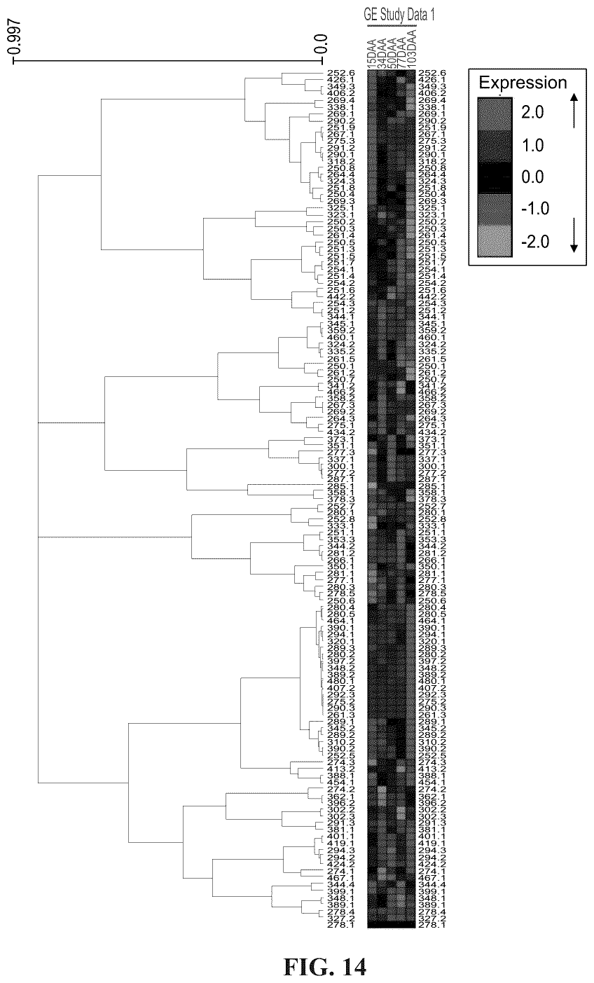

In order to identify the UGT family enzymes responsible for mogrol glucosylation, nearly 100 genes of the total about 160 UGTs in the Siraitia genome (FIGS. 13A and 13B) which showed expression in the developing fruit (FIG. 14) were functionally expressed in E. coli as described above. The extracted recombinant enzymes were assayed with 0.1 mM of each of the 10 substrates (M, M1-E1, M2-A1, M2Δ, M2-E, M3×, M3, Siamenoside, M4, and M5), and 8 mM UDP-glucose, as glucose donor.

The overall results for the screening are presented in the activity matrices in FIGS. 15A-15C. The results identified three genes that carried out strictly the primary C3 glucosylation, members of UGT families 74, 75 and 85 (FIG. 15A columns A-D). A fourth gene, UGT85E5 (SEQ ID NO: 33) was the only identified gene capable of strictly carrying out the specific C24 primary glucosylation (FIG. 15A, C1). Additional enzymes of the UGT73 family were identified which carried either C25 glucosylation or a mix of C24 and C25 glucosylation (FIG. 15A, columns E-G), as identified by NMR.

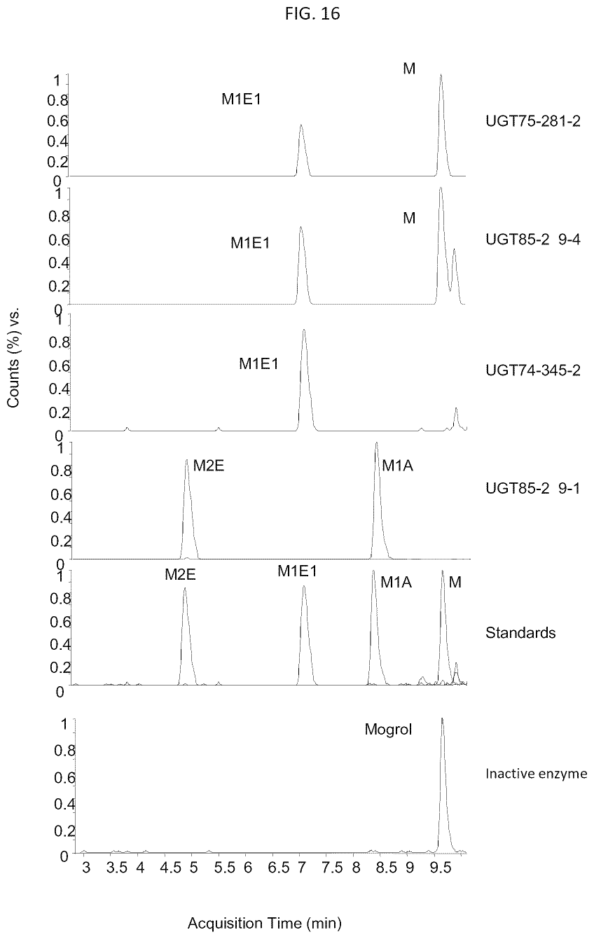

Significantly, UGT85-269-1 was not only capable of carrying out the primary C-24 glucosylation of mogrol, but subsequently also the C-3 primary glucosylation of C-24-glucosylated mogrol, thus accounting itself for the synthesis of the diglucosylated M2. Thus, the UGT85-269-1 enzyme yielded both M1-C24 and M2-C3, C24 when incubated with mogrol, but not M1-C3 (FIG. 15A, C2-3, FIG. 16). It can furthermore be seen in FIG. 15A that the enzymes performing primary C3 glucosylation are also capable of performing the reaction irrespective of the glucosylation status C24, whether 0, 1, 2 or 3 glucose moieties occupy the position (FIG. 15A, columns A-D, rows 2-6).

### Branched Glucosylations

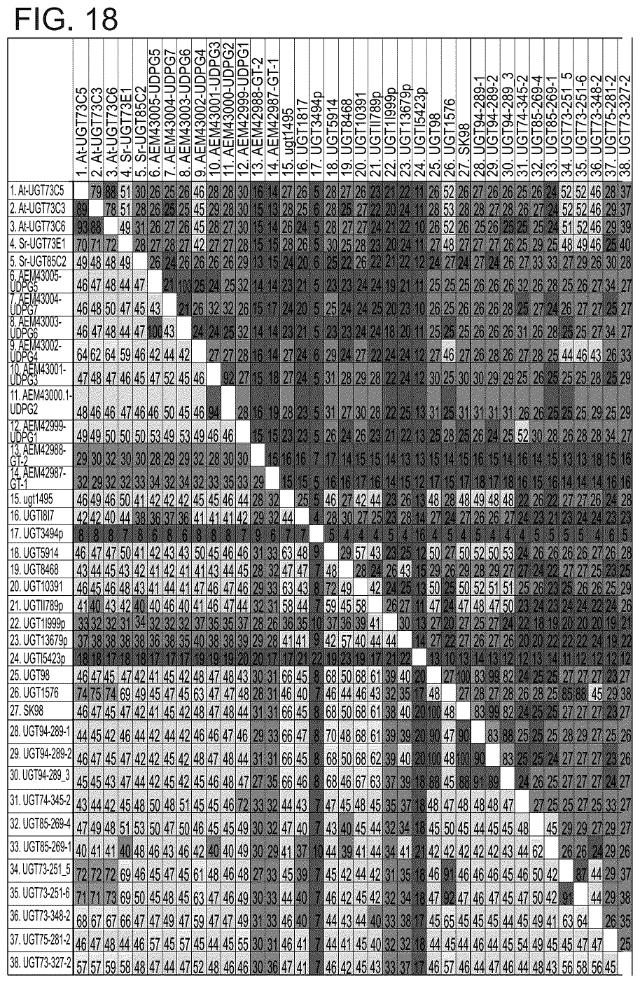

The subsequent secondary branching glycosylations were carried out by three members of a single UGT family, UGT94, which were specific for branching and did not perform primary glucosylations (FIG. 15B columns I,J,K; FIG. 15C, columns M,N,O). The three UGT94 enzymes show differences in substrate specificity and activity as depicted in FIGS. 15B and 15C. UGT94 (289-3) and UGT94 (289-1) appear to be the most versatile, each leading to the pentaglucosylated M5 from M4, while UGT94 (289-2), appears to be most limited in its substrate specificity. FIG. 18 shows the similarity and identity scores between each of the genes described herein and the prior known gene sequences from Siraitia, described in Tang et al (2011) and WO2013/076577. The matrix was determined using MatGAT 2.02 (www(dot)bitincka(dot)com/ledion/matgat/) run with BLOSUM62.

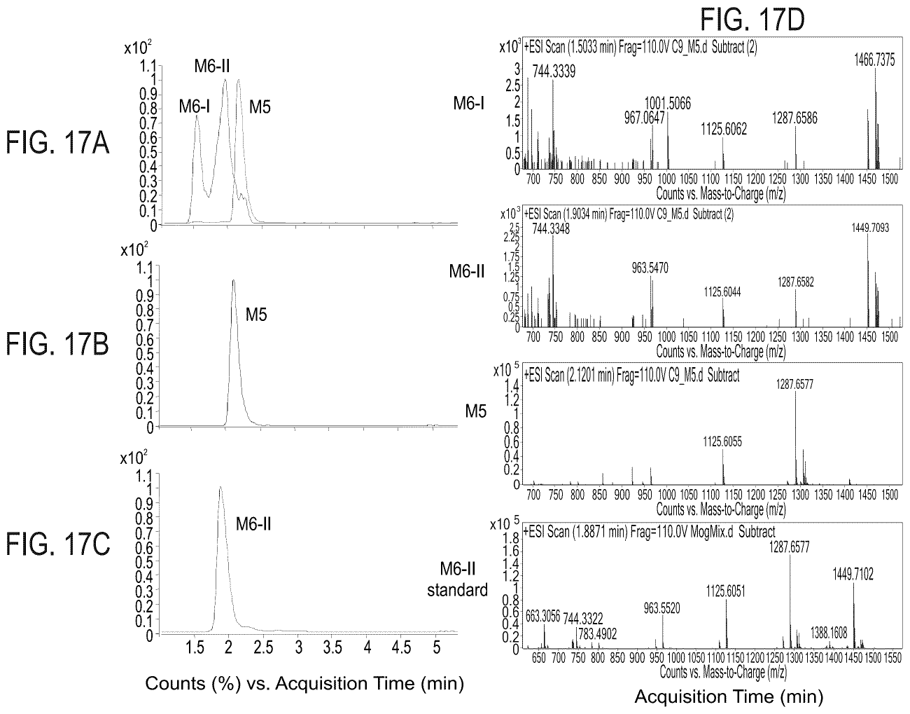

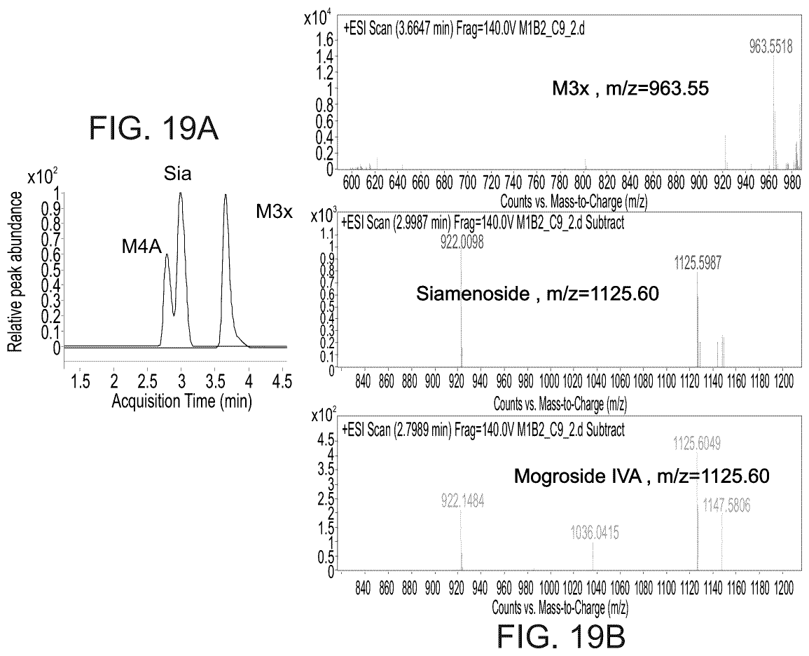

Surprisingly, in some of the reactions of UGT94 (289-3) with M5 as substrate we observed an M6 product (m/z 1642.5) (FIG. 17A). Furthermore, the branching enzyme UGT94 (289-3) was also capable of carrying out consecutive reactions of branching (FIG. 19A). When M1A1 was incubated with both UGT74-345-2 and UGT94-289-3 we observed M4 products. Since UGT94-289-3 can produce M5 from M4 substrates, as depicted in FIG. 15B, without wishing to be limited to a single hypothesis, it is possible that UGT94-289-3 can carry out the complete array of branching reactions if supplied with adequate substrate and optimal reaction conditions.

Surprisingly, UGT85E5 also showed branching activity, specifically on the C-3 primary glucose (FIG. 15B, column H)) and it too may contribute to the branching portion of the pathway, making it a key enzyme in mogroside synthesis.

In summary, based on the combined metabolic profiling, functional expression and protein modeling results the following metabolic pathway for mogroside biosynthesis is conceivable. During the initial stage of fruit development squalene is metabolized to the diglucosylated M2, via the progressive actions of squalene synthase, squalene epoxidase, cucurbitadienol synthase, epoxide hydrolase, cytochrome p450 (cyp102801) and UGT85. During fruit maturation there is the progressive activity of the UGT94 members, and perhaps also the UGT85, adding branched glucosyl groups to the primary glucosyl moieties of M2, leading to the sweet-flavored M4, M5 and M6 compounds.

The individual reactions summarized in FIGS. 15A-15C are described in the following individual examples.

### Example 6

### UGT74-345-2 Catalyzes the Addition of the Primary Glucose at Position C3

Reaction containing UGT74-345-2 recombinant enzyme provided 0.1 mM aglycone Mogrol as substrate and 8 mM UDP-Glucose as sugar donor resulted in accumulation of MI-E1 (FIG. 15A-A1), whilst the same reaction containing 0.1 mM of MI-A1 as a substrate, resulted in accumulation of MII-E (FIG. 15A-A2). Moreover, in reaction containing 0.1 mM of M2-A1 accumulation of M3x was measured and in that containing MII-A accumulation of M3 was observed (FIG. 15A-A4 and A5). Furthermore, in the presence of MIII-A1 siamenoside was produced (FIG. 15A-A6). The analysis of the products of those reactions points to ability of UGT74-345-2 to perform primary glucosylation, attaching glucose moiety on C-3 position of Mogrol/Mogroside.

### UGT75-281-2 Catalyzes the Addition of the Primary Glucose at Position C3.

Reaction containing UGT75-281-2 recombinant enzyme provided 0.1 mM aglycone Mogrol as substrate and 8 mM UDP-Glucose as sugar donor resulted in accumulation of MI-E1 (FIG. 15A-B1 and FIG. 16), whilst the same reaction containing 0.1 mM of MI-A1 as a substrate, resulted in accumulation of MII-E (FIG. 15A-B2 and FIG. 16). Moreover, in a reaction containing 0.1 mM of M2-A1 accumulation of M3x was measured and in that containing MII-A accumulation of M3 was observed (FIG. 15A-B4 and B5). The analysis of the products of those reactions points to ability of UGT75-281-2 to perform primary glucosylation, attaching glucose moiety on C-3 position of Mogrol/Mogroside.

### UGT85-269-1 is a Promiscuous Enzyme and Catalyzes the Primary and the Branched Addition of Glucose

Using 0.1 mM M, M1A1, M1E1, M2A1 or M2A as a substrate and 8 mM UDP-Glucose as sugar donor, accumulation of M1A1, M2E, M2E, M3x or M3, respectively, was observed when UGT85-269-1 recombinant enzyme was added into reaction (FIG. 15A-C1-C5 and FIG. 16). Therefore the UGT85-269-1 is a primary glucosyltransferase from Mogroside biosynthetic pathway, and is able to attach glucose (glucosylate) at C-3 or C-24 of Mogrol/mogroside. Given M2E, M3, M3x or Siamenoside as a substrate, UGT269-1-containing reaction mixes accumulated putative M3-C3 (1-6), isomogroside 4 and trace amounts of M4, M4A and isomogroside 5, respectively (FIG. 15B-H1-H3 and H4). Indicating that UGT85-269-1 can act as both a primary and branched glucosyltransferase from Mogroside biosynthetic pathway.

### UGT85-269-4 Catalyzes the Addition of the Primary Glucose at Position C3

Using 0.1 mM M, M1A1 M2A1 or M2A as a substrate and 8 mM UDP-Glucose as sugar donor, accumulation of M1E1, M2E, M3x or M3, respectively, was observed when UGT85-269-4 recombinant enzyme was added into reaction (FIG. 15A-D1-D5 and FIG. 16). Therefore the UGT85-269-4 is a primary glucosyltransferase from Mogroside biosynthetic pathway, and is able to attach glucose (glucosylate) at the C-3 position of mogrol.

### UGT73-251-5 Catalyzes the Addition of the Primary Glucose at Position C24 or C25

When the UGT73-251-5 recombinant enzyme was added to a reaction mix containing 0. 1 mM aglycone Mogrol as substrate and 8 mM UDP-Glucose as sugar donor, accumulation of M1-A1 and M1-B (FIG. 15A-E1) was observed, suggesting that UGT73-251-5 acts as C-24 and C-25 glucosyltransferase.

### UGT73-251-6 Catalyzes the Addition of the Primary Glucose at Position C25

When the UGT73-251-6 recombinant enzyme was added to a reaction mix containing 0.1 mM aglycone Mogrol as substrate and 8 mM UDP-Glucose as sugar donor, accumulation of M1-B (FIG. 15A-D1) was observed, suggesting that UGT73-348-2 is C-glucosyltransferase.

### UGT73-348-2 Catalyzes the Addition of the Primary Glucose at Position C24

^{a}When the UGT73-348-2 recombinant enzyme was added to a reaction mix containing 0.1 mM aglycone Mogrol as substrate and 8 mM UDP-Glucose as sugar donor, accumulation of M1-A1 and M1-B (FIG. 15A-G1) was observed, suggesting that UGT73-348-2 is C-24 and C-25 glucosyltransferase.

### UGT94-289-1 Catalyzes the Branched Additions of Glucose to the Primary Glucose at Position C24 and C3 in a 1-6 Position

Using 0.1 mM Mogroside IIE as a substrate and 8 mM UDP-Glucose as sugar donor, accumulation of M3x was observed when UGT94-289-1 recombinant enzyme was added into reaction (FIG. 15B-K1). When M3 was used as a substrate, Siamenoside and trace amount of M4 accumulated in the reaction mix (FIG. 15B-K2). Finally, when M4 was used as a substrate, M5 was found to accumulate in reaction mix (FIG. 15B-K4). In addition, when M1A1, M2A1 or M2A were added as substrate for glucosylation, M2A1, M3-A1 and M3-A1 accumulated, respectively (FIG. 15C-O1, O3 and O4). Therefore the UGT94-289-1 is a branching glucosyltransferase from Mogroside biosynthetic pathway, and is able to attach glucose at (1-6) and (1-2) position on C-24 and C-3 glucosylated mogroside.

### UGT94-289-2 Catalyzes the Branched Additions of Glucose to the Primary Glucose at Position C24 in a 1-6 Position

Using 0.1 mM Mogroside IIE as a substrate and 8 mM UDP-Glucose as sugar donor, accumulation of M3x was observed when UGT94-289-2 recombinant enzyme was added into reaction (FIG. 15B-J1), whilst when M3 was used as substrate, accumulation of Siamenoside was observed in reaction mix (FIG. 15B-J2). In addition, when M1A1 or M2A were added as substrate for glucosylation, M2A1 and M3-A1 accumulated, respectively (FIG. 15C-N1 and N4). Therefore the UGT94-289-2 is a branching glucosyltransferase from Mogroside biosynthetic pathway, and is able to attach glucose at (1-6) position on C-24 glucosylated mogroside.

### UGT94-289-3 is a Promiscuous Enzyme Catalyzes the Branched Additions of Glucose to the Primary Glucose at Position C24 and C3 in a 1-6 or 1-2 Position

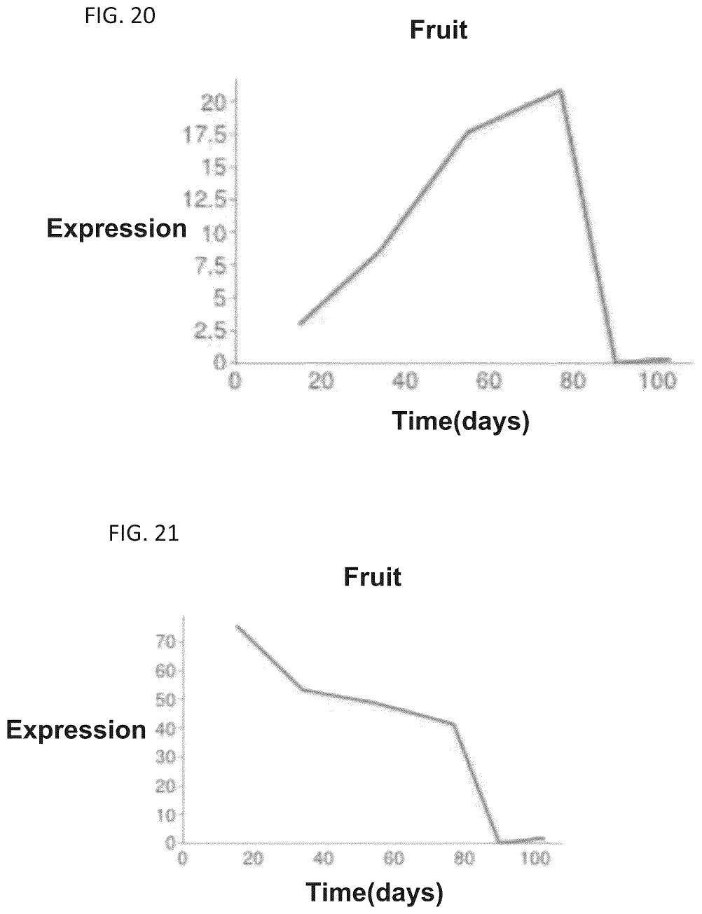

Using 0.1 mM Mogroside IIE as a substrate and 8 mM UDP-Glucose as sugar donor, accumulation of M3x was observed when UGT94-289-3 recombinant enzyme was added into reaction (FIG. 15B-I1). When M3, M3x M4 or Siamenoside were used as substrates, Siamenoside (with trace amounts of M4), M4A with Siamenoside, M5 and M5 were found in reaction mix, respectively (FIG. 15B-I2-I5 and FIG. 20). In addition, when M1A1, M1E1, M2Δ1 or M2 were added as substrate for glucosylation, M2Δ1, M2-A2, M3-A1 and M3-A1 accumulated, respectively (FIG. 165-M1-M4). Therefore the UGT94-289-3 is branching glucosyltransferase from Mogroside biosynthetic pathway, and is able to attach glucose at (1-6) and (1-2) positions on C-24 or C-3 glucosylated mogroside. In some of the reactions of UGT94-289-3 with M5 as substrate we observed an M6 product (m/z 1449.7113) (FIG. 15B-I6 and FIG. 17A).

UGT73-327-2 Catalyzes the Branched Addition of Glucose to the Primary Glucose at Position C3 in a 1-2 Position to Yield M6 from M5

Enzyme UGT73-327-2 was found to catalyze the final step in biosynthesis of Mogroside VI. When heterologously expressed UGT73-327-2 protein was added to reaction containing 0.1 mM Mogroside V and 8 mM UDP-Glucose, Mogroside VI was found among the reaction products, therefore designating UGT73-327-2 as a likely (1-2) C-3-Glu glucosyltransferase (FIG. 15B-L6).

### Example 7

### Phylogenetic Tree of the UGT Enzymes

Similarity and identity scores between each of the genes described herein and the nine prior known gene sequences from Siraitia were determined using MatGAT 2.02 (www(dot)bitincka(dot)com/ledion/matgat/) run with BLOSUM62. FIGS. 13A-B describe phylogenetic trees of the currently known UGTs as well as the novel UGTs of some embodiments of the invention. Alignments were carried out using the Clustal X software using default settings. Bootstrap values were also carried out using the Clustal X software (1000 iterations). The tree was visualized using the NJPLOT software. Numbers on tree branches show bootstrap proportions, which are the frequencies with which groups are encountered in analyses of replicate data sets and therefore provide an index of support for those groups. The length of the branches correspond to the numbers of substitutions per site.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. An isolated uridine diphospho-glucosyl transferase enzyme (UGT) polypeptide wherein said polypeptide catalyzes glucosylation of mogrol or mogroside and wherein said polypeptide is selected from the group consisting of:
a polypeptide having an amino acid sequence at least 90% identical to SEQ ID NO: 2, a polypeptide having an amino acid sequence at least 90% identical to SEQ ID NO: 4, a polypeptide having an amino acid sequence identical to SEQ ID NO: 6, a polypeptide having an amino acid sequence at least 90% identical to SEQ ID NO: 8, a polypeptide having an amino acid sequence at least 90% identical to SEQ ID NO: 26, a polypeptide having an amino acid sequence at least 90% identical to SEQ ID NO: 28, a polypeptide having an amino acid sequence at least 90% identical to SEQ ID NO: 30, a polypeptide having an amino acid sequence at least 90% identical to SEQ ID NO: 32, a polypeptide having an amino acid sequence at least 90% identical to SEQ ID NO: 36 and a polypeptide having an amino acid sequence at least 90% identical to SEQ ID NO: 38.

2. An isolated polynucleotide comprising a nucleic acid sequence encoding the polypeptide of claim 1.

3. The isolated polynucleotide of claim 2, wherein said polypeptide has an amino acid sequence at least 90% identical to SEQ ID NO: 2

4. The isolated polynucleotide of claim 2, wherein said polypeptide has an amino acid sequence at least 90% identical to SEQ ID NO: 4.

5. The isolated polynucleotide of claim 2, wherein said polypeptide has an amino acid sequence identical to SEQ ID NO: 6.

6. The isolated polynucleotide of claim 2, wherein said polypeptide has an amino acid sequence at least 90% identical to SEQ ID NO: 8.

7. The isolated polynucleotide of claim 2, wherein said polypeptide has an amino acid sequence at least 90% identical to SEQ ID NO: 26.

8. The isolated polynucleotide of claim 2, wherein said polypeptide has an amino acid sequence at least 90% identical to SEQ ID NO: 28.

9. The isolated polynucleotide of claim 2, wherein said polypeptide has an amino acid sequence at least 90% identical to SEQ ID NO: 30.

10. The isolated polynucleotide of claim 2, wherein said polypeptide has an amino acid sequence at least 90% identical to SEQ ID NO: 32.

11. The isolated polynucleotide of claim 2, wherein said polypeptide has an amino acid sequence at least 90% identical to SEQ ID NO: 36.

12. The isolated polynucleotide of claim 2, wherein said polypeptide having an amino acid sequence at least 90% identical to SEQ ID NO: 38.

13. A host cell heterologously expressing the isolated polypeptide of claim 1.

14. The host cell of claim 13, further expressing a polynucleotide having the nucleotide sequence of SEQ ID NO: 33.

15. The host cell of claim 13, expressing at least two of:
(i) a polynucleotide encoding a polypeptide having an amino acid sequence at least 90% identical to SEQ ID NO: 2;
(ii) a polynucleotide encoding a polypeptide having an amino acid sequence at least 90% identical to SEQ ID NO: 4;
(iii) a polynucleotide encoding a polypeptide having an amino acid sequence identical to SEQ ID NO: 6;
(iv) a polynucleotide encoding a polypeptide having an amino acid sequence at least 90% identical to SEQ ID NO: 8;
(v) a polynucleotide encoding a polypeptide having an amino acid sequence at least 90% identical to SEQ ID NO: 26;
(vi) a polynucleotide encoding a polypeptide having an amino acid sequence at least 90% identical to SEQ ID NO: 28;
(vii) a polynucleotide encoding a polypeptide having an amino acid sequence at least 90% identical to SEQ ID NO: 30;
(viii) a polynucleotide encoding a polypeptide having an amino acid sequence at least 90% identical to SEQ ID NO: 32;
(ix) a polynucleotide encoding a polypeptide having an amino acid sequence at least 90% identical to SEQ ID NO: 34;
(x) a polynucleotide encoding a polypeptide having an amino acid sequence at least 90% identical to SEQ ID NO: 36, and
(xi) a polynucleotide encoding a polypeptide having an amino acid sequence at least 90% identical to SEQ ID NO: 38.

16. The host cell of claim 15, expressing a combination of polynucleotides selected from the group consisting of (iii) and (iv); (iii) and (ix); (iii) and (xi); (iv) and (ix); (iv) and (xi); (ix) and (xi); (iii) and (iv) and (ix); (iii) and (iv) and (xi); (iii) and (ix) and (xi); (iv) and (ix) and (xi); and (iii) and (iv) and (ix) and (xi).

17. The host cell of claim 13, wherein said host cell is selected from the group consisting of a yeast cell, a bacteria and a plant cell.

18. A nucleic acid construct comprising the isolated polynucleotide of claim 2 and a cis-acting regulatory element for directing expression of the isolated polynucleotide.

19. A cell lysate of the host cell of claim 13.

20. A method of synthesizing a mogroside, the method comprising contacting the isolated UGT polypeptide of claim 1 with at least one mogrol or mogroside UGT substrate.


SEQUENCE LISTING (59 sequences)

SEQ ID NO: 1 - DNA, 1647 bp, Siraitia grosvenori
  source 1..1647: mol_type = genomic DNA
atggacgaaaattataattttttaaaagttcaaaccttcaatctcaagaaattcaacaaaaattttccatcgcaacgacgccgttcacctgcaccaagtaactacttcatgtgtacttcacttgctgaattccttccgtgtaaaacgtggttccagtttgtctcagcaatattctatttaaatggtccttcgcacgatttccaccatttgcctgctgcaccgggcggcgattccgacggaagagagaagagccgatcgccggaaatggacgagacgacggtgaacggagggcggagagcgagtgatgtggtggtgttcgctttcccgaggcacggccatatgagtccgatgctccaattctccaagcgtttggtctccaaaggcctccgcgtcacgtttctcatcaccacctctgcaactgaatccctccgactaaatcttcctccctcttcttctctcgatcttcaagttatctccgacgtccctgaaagtaacgacatcgcgacgctcgaagggtatcttcgaagcttcaaggccactgtttccaaaaccttggcggatttcatcgacggaatcggaaatcctccaaagttcatcgtttacgattcggtcatgccgtgggtgcaggaggtagccagagggcgcggcctcgatgcggcgccgtttttcactcaatcgtccgccgttaatcacatcctcaatcatgtgtacggaggatctttgagcattccggccccggagaacacggcagtttcgcttccttcgatgccggttcttcaagccgaggatctgccggccttccccgacgacccagaagtggttatgaacttcatgaccagtcaattctccaatttccaggatgcaaaatggattttcttcaacacattcgatcagctggagtgcaagaaacaaagtcaggttgttaattggatggccgacagatggcccatcaagacagtgggaccgaccattccatcggcatatttggacgacggtcggttggaggatgacagggcgtttggtctgaatctcctaaaacctgaagatgggaagaacactaggcagtggcagtggttagactcaaaagacactgcttctgtcctttatatttcatttggaagcttggctatcttacaagaagaacaagtgaaggaactggcatatttcctcaaagacaccaatctttccttcttatgggtccttagagactcagaactccaaaagcttccccacaactttgtacaagagacatcacacagaggtctggttgtaaactggtgctctcaactacaagttctgtctcacagggctgtaagttgctttgtgactcattgtggttggaattcgacgctcgaagctttgagcttgggggtgccgatggtcgcaattccacagtgggttgatcaaacgacaaacgccaagttcgttgcagatgtttggagagtgggagttagagtgaagaagaaggacgaacgcatcgtaaccaaggaagaactagaagcctccatccgacaggttgttcaaggagaggggagaaatgagtttaaacataatgcaatcaagtggaagaagctggctaaagaagcagtggatgaaggtggcagctctgataaaaacattgaagaatttgtcaagacaattgcatga

SEQ ID NO: 2 - protein, 456 aa, Siraitia grosvenori
MDETTVNGGRRASDVVVFAFPRHGHMSPMLQFSKRLVSKGLRVTFLITTSATESLRLNLPPSSSLDLQVISDVPESNDIATLEGYLRSFKATVSKTLADFIDGIGNPPKFIVYDSVMPWVQEVARGRGLDAAPFFTQSSAVNHILNHVYGGSLSIPAPENTAVSLPSMPVLQAEDLPAFPDDPEVVMNFMTSQFSNFQDAKWIFFNTFDQLECKVVNWMADRWPIKTVGPTIPSAYLDDGRLEDDRAFGLNLLKPEDGKNTRQWQWLDSKDTASVLYISFGSLAILQEEQVKELAYFLKDTNLSFLWVLRDSELQKLPHNFVQETSHRGLVVNWCSQLQVLSHRAVSCFVTHCGWNSTLEALSLGVPMVAIPQWVDQTTNAKFVADVWRVGVRVKKKDERIVTKEELEASIRQVVQGEGRNEFKHNAIKWKKLAKEAVDEGGSSDKNIEEFVKTIA

SEQ ID NO: 3 - DNA, 1479 bp, Siraitia grosvenori
  source 1..1479: mol_type = genomic DNA
atggcttctcttctcagccagattcacattgttgtgattccattgatgactcaaggtcacctgatccctgcagccgacatggcaaagctattggcagagcgcggcgttaccgtcactatcatcaccacccctctcaacgccaagcggattcagacgctcgttgatcgcgctcgagaggccaatctcgatctccgacttgtcgaccgactcaacattccgctcgctgagttcggcttgccggaagggtgtgagagcgtagatcgagtcccctcgcgggaactgtttaagaatttctttttggctctcaacgacttgcaaaaacccctcgagaggctcgtcgctaggttgcaaccacgccccagctgcgtaattgctgataaaaatctgccatgggtggtaggtgtttgtgaaaagttccaagtgacgaggttttcgtttgatggcactagttgtttttctctgttatgttctaacaacatacgtgcgtctaaggtcctcgagagtgtgaattcggtatcagagagctttttggttcctgggttacctgataggattgaagttactgcagctcaattaccagcagacttgaatccaggttcatatttaaaagagctacatgaaagtggaagaattgctcatgagaatgcctatgggttgctggttaatagtttcgaggagttggaatctgaatacttgaaggaatatcgaaaggtgaaaggcgataaaatctggtgcattggccctgtgtccctatccaataagacaggcgtggaaagggcccaacgcggcggcatagccgcacaagatgctgacaagtgcttgcgctggctcgattcatggcctaagagctctgttgtttacgtttgtattgggagcctcagccggctttcatctcaacaaagttcagagcttgctttagccttagaagaatcaaaccaaccattcatttgggtcgtaaaggaagaggagaagcatgaaacatcaaagactacatcgacggtcggatccatgagagcttttgaagaaaggacgaagggaagagggattctgctgaagggttgggctccgcagctgcagatcttgtcgcacccggccgtcggagcatttctaacccactgcggctggaactctgttctggaaggcgtctgcgccggtgttccattgatcacatggcccatgttcgccgaccagttctttaacgagaaggaagttgttgaagttttgaagattggggaagaagttggaaacaagaagacggtgccattgggggatgaagagaagagcgaggtggtgatcggcagagaggagattaagaaggctattggtgcggtaatggaggaaggagcagaggcagagagaagaaagagagcgagaaggctggctgagaaggcaaacaaagctatggaagatggggggagttcttatgttaatgttacacgattggttgaacatatcaagcaactggtgcttgaaaaatga

SEQ ID NO: 4 - protein, 492 aa, Siraitia grosvenori
MASLLSQIHIVVIPLMTQGHLIPAADMAKLLAERGVTVTIITTPLNAKRIQTLVDRAREANLDLRLVDRLNIPLAEFGLPEGCESVDRVPSRELFKNFFLALNDLQKPLERLVARLQPRPSCVIADKNLPWVVGVCEKFQVTRFSFDGTSCFSLLCSNNIRASKVLESVNSVSESFLVPGLPDRIEVTAAQLPADLNPGSYLKELHESGRIAHENAYGLLVNSFEELESEYLKEYRKVKGDKIWCIGPVSLSNKTGVERAQRGGIAAQDADKCLRWLDSWPKSSVVYVCIGSLSRLSSQQSSELALALEESNQPFIWVVKEEEKHETSKTTSTVGSMRAFEERTKGRGILLKGWAPQLQILSHPAVGAFLTHCGWNSVLEGVCAGVPLITWPMFADQFFNEKEVVEVLKIGEEVGNKKTVPLGDEEKSEVVIGREEIKKAIGAVMEEGAEAERRKRARRLAEKANKAMEDGGSSYVNVTRLVEHIKQLVLEK

SEQ ID NO: 5 - DNA, 1362 bp, Siraitia grosvenori
  source 1..1362: mol_type = genomic DNA
atggatgcccagcgaggtcacaccacaaccattttgatgtttccatggctcggctatggccatctttcggctttcctagagttggccaaaagcctctcaaggaggaacttccatatctacttctgttcaacctctgttaacctcgacgccattaaaccaaagcttccttcttcttcctcttctgattccatccaacttgtggaactttgtcttccatcttctcctgatcagctccctcctcatcttcacacaaccaacgccctcccccctcacctcatgcccactctccaccaagccttctccatggctgcccaacactttgctgccattttacacacacttgctccgcatctcctcatttacgactctttccaaccttgggctcctcaactagcttcatccctcaacattccagccatcaacttcaatactacgggagcttcagtcctgacccgaatgcttcacgctactcactacccaagttctaaattcccaatttcagagtttgttctccacgattattggaaagccatgtacagcgccgccggtggggctgttacaaaaaaagaccacaaaattggagaaacacttgcgaattgcttgcatgcttcttgtagtgtaattctaatcaatagtttcagagagctcgaggagaaatatatggattatctctccgttctcttgaacaagaaagttgttccggttggtcctttggtttacgaaccgaatcaagacggggaagatgaaggttattcaagcatcaaaaattggcttgacaaaaaggaaccgtcctccaccgtcttcgtttcatttggaagcgaatacttcccgtcaaaggaagaaatggaagagatagcccatgggttagaggcgagcgaggttcatttcatctgggtcgttaggtttcctcaaggagacaacaccagcgccattgaagatgccttgccgaaggggtttctggagagggtgggagagagagggatggtggtgaagggttgggctcctcaggcgaagatactgaagcattggagcacagggggattcgtgagccactgtggatggaactcggtgatggaaagcatgatgtttggcgttcccataataggggttccgatgcatctggaccagccctttaacgccggactcgcggaagaagctggcgtcggcgtggaggccaagcgagatccagacggcaaaattcaaagagacgaagttgcaaagttgatcaaagaagtggtggttgagaaaaccagagaagacgtgcggaagaaagcaagagaaatgagtgagattttgaggagcaaaggagaggagaagatggatgagatggtggctgcaatctctctctttcttaaaatatga

SEQ ID NO: 6 - protein, 453 aa, Siraitia grosvenori
MDAQRGHTTTILMFPWLGYGHLSAFLELAKSLSRRNFHIYFCSTSVNLDAIKPKLPSSSSSDSIQLVELCLPSSPDQLPPHLHTTNALPPHLMPTLHQAFSMAAQHFAAILHTLAPHLLIYDSFQPWAPQLASSLNIPAINFNTTGASVLTRMLHATHYPSSKFPISEFVLHDYWKAMYSAAGGAVTKKDHKIGETLANCLHASCSVILINSFRELEEKYMDYLSVLLNKKVVPVGPLVYEPNQDGEDEGYSSIKNWLDKKEPSSTVFVSFGSEYFPSKEEMEEIAHGLEASEVHFIWVVRFPQGDNTSAIEDALPKGFLERVGERGMVVKGWAPQAKILKHWSTGGFVSHCGWNSVMESMMFGVPIIGVPMHLDQPFNAGLAEEAGVGVEAKRDPDGKIQRDEVAKLIKEVVVEKTREDVRKKAREMSEILRSKGEEKMDEMVAAISLFLKI

SEQ ID NO: 7 - DNA, 1476 bp, Siraitia grosvenori
  source 1..1476: mol_type = genomic DNA
atgggctccgccggcgtcgaactgaaggtggctttcctgccatttgcagctccaggtcacatgattcccttgatgaacatagccagactcttcgccatgcacggcgccgacgtcaccttcatcaccaccccggccactgcctcccgcttccaaaacgtcgtcgactccgatctccgacgcggccacaaaatcaaactccatacatttcaactgccctctgcagaagccggtctcccccccggcgtcgagagcttcaacgaatgcacttctaaagagatgaccgaaaaactcttcggcgcatttgaaatgctcaacggagacatcgaacagttcctcaaaggggctaaagtcgactgcattgtgagcgatacgattctcgtttggaccttggacgccgctgcaaggctcgggattccgaggatagctttccgatcttcaggattcttttcggaatgtattcatcactctttaaggtgtcacaagcctcacaagaaggtgggatccgatacagagccgtttatatttcctggtttaccgcataagattgagataacgagattgaatataccacaatggtattcagaagaaggctatattcagcatattgaaaagatgaaagaaatggacaaaaagagttatgcggtactgctaaataccttctatgagcttgaggctgattatgttgaatattttgaatctgttattgggttgaaaacatggatcgtagggccagtttccttatgggctaacgagggtggaggcaaaaacgactcaagaactgagaacaacaacgctgagttgatggaatggctggactccaaacagcctaattcagttctgtatgttagttttggtagcatgacgaagttcccatctgctcaggtgctcgaaatagctcacggccttgaagattctggctgccatttcatttgggtggttcgaaagatgaacgaaagtgaagcagctgatgaagaatttccagaggggttcgaggagagagtgagagagagcaagagaggtttgatcataagagattgggcgccgcaagaattgattttgaatcatgcagctgttggggggtttgtcactcactgtggctggaattcaattctcgaaagtgtatgtgctggtcggccgatcatcgcgtggccgttgtcggcggaacagtttttcaacgagaagtttgtaactcgtgtattgaaagttggagtttcaattggtgtaagaaaatggtggggctcgacgagttcagaaactttagatgtggtgaaaagggatcgaattgcagaagcagtggcgaggttgatgggagatgacagagaggtggttgaaatgagagatggagttagagaactttcacatgcagcgaagagagcaataaaggaaggtggatcttctcactcaaccttgctctcattgatccatgaactcaagaccatgaaatttaaacgccaaagtagtaatgtggatggataa

SEQ ID NO: 8 - protein, 491 aa, Siraitia grosvenori
MGSAGVELKVAFLPFAAPGHMIPLMNIARLFAMHGADVTFITTPATASRFQNVVDSDLRRGHKIKLHTFQLPSAEAGLPPGVESFNECTSKEMTEKLFGAFEMLNGDIEQFLKGAKVDCIVSDTILVWTLDAAARLGIPRIAFRSSGFFSECIHHSLRCHKPHKKVGSDTEPFIFPGLPHKIEITRLNIPQWYSEEGYIQHIEKMKEMDKKSYAVLLNTFYELEADYVEYFESVIGLKTWIVGPVSLWANEGGGKNDSRTENNNAELMEWLDSKQPNSVLYVSFGSMTKFPSAQVLEIAHGLEDSGCHFIWVVRKMNESEAADEEFPEGFEERVRESKRGLIIRDWAPQELILNHAAVGGFVTHCGWNSILESVCAGRPIIAWPLSAEQFFNEKFVTRVLKVGVSIGVRKWWGSTSSETLDVVKRDRIAEAVARLMGDDREVVEMRDGVRELSHAAKRAIKEGGSSHSTLLSLIHELKTMKFKRQSSNVDG

SEQ ID NO: 9 - DNA, 1422 bp, Siraitia grosvenori
  source 1..1422: mol_type = genomic DNA
atgtggactgtcgtgctcggtttggcgacgctgtttgtcgcctactacatccattggattaacaaatggagagattccaagttcaacggagttctgccgccgggcaccatgggtttgccgctcatcggagagacgattcaactgagtcgacccagtgactccctcgacgttcaccctttcatccagaaaaaagttgaaagatacgggccgatcttcaaaacatgtctggccggaaggccggtggtggtgtcggcggacgcagagttcaacaactacataatgctgcaggaaggaagagcagtggaaatgtggtatttggatacgctctccaaatttttcggcctcgacaccgagtggctcaaagctctgggcctcatccacaagtacatcagaagcattactctcaatcacttcggcgccgaggccctgcgggagagatttcttccttttattgaagcatcctccatggaagcccttcactcctggtctactcaacctagcgtcgaagtcaaaaatgcctccgctctcatggtttttaggacctcggtgaataagatgttcggtgaggatgcgaagaagctatcgggaaatatccctgggaagttcacgaagcttctaggaggatttctcagtttaccactgaattttcccggcaccacctaccacaaatgcttgaaggatatgaaggaaatccagaagaagctaagagaggttgtagacgatagattggctaatgtgggccctgatgtggaagatttcttggggcaagcccttaaagataaggaatcagagaagttcatttcagaggagttcatcatccaactgttgttttctatcagttttgctagctttgagtccatctccaccactcttactttgattctcaagctccttgatgaacacccagaagtagtgaaagagttggaagctgaacacgaggcgattcgaaaagctagagcagatccagatggaccaattacttgggaagaatacaaatccatgacttttacattacaagtcatcaatgaaaccctaaggttggggagtgtcacacctgccttgttgaggaaaacagttaaagatcttcaagtaaaaggatacataatcccggaaggatggacaataatgcttgtcaccgcttcacgtcacagagacccaaaagtctataaggaccctcatatcttcaatccatggcgttggaaggacttggactcaattaccatccaaaagaacttcatgccttttgggggaggcttaaggcattgtgctggtgctgagtactctaaagtctacttgtgcaccttcttgcacatcctctgtaccaaataccgatggaccaaacttgggggaggaaggattgcaagagctcatatattgagttttgaagatgggttacatgtgaagttcacacccaaggaatga

SEQ ID NO: 10 - protein, 473 aa, Siraitia grosvenori
MWTVVLGLATLFVAYYIHWINKWRDSKFNGVLPPGTMGLPLIGETIQLSRPSDSLDVHPFIQKKVERYGPIFKTCLAGRPVVVSADAEFNNYIMLQEGRAVEMWYLDTLSKFFGLDTEWLKALGLIHKYIRSITLNHFGAEALRERFLPFIEASSMEALHSWSTQPSVEVKNASALMVFRTSVNKMFGEDAKKLSGNIPGKFTKLLGGFLSLPLNFPGTTYHKCLKDMKEIQKKLREVVDDRLANVGPDVEDFLGQALKDKESEKFISEEFIIQLLFSISFASFESISTTLTLILKLLDEHPEVVKELEAEHEAIRKARADPDGPITWEEYKSMTFTLQVINETLRLGSVTPALLRKTVKDLQVKGYIIPEGWTIMLVTASRHRDPKVYKDPHIFNPWRWKDLDSITIQKNFMPFGGGLRHCAGAEYSKVYLCTFLHILCTKYRWTKLGGGRIARAHILSFEDGLHVKFTPKE

SEQ ID NO: 11 - DNA, 2277 bp, synthetic construct
  misc_feature 1..2277: note = Optimised Nucleic Acid sequence encoding SgCDS cucurbitadienol synthase
  source 1..2277: mol_type = other DNA
atgtggaggttaaaggtcggagcggaatccgttggtgagaacgacgaaaaatggttaaagtcaatatcaaaccatttgggtaggcaagtttgggaattttgtccagatgcgggtactcaacaacagctgctacaagttcataaagcacgtaaagctttccacgatgaccgtttccacagaaaacagtcatcagacttgttcatcacgatccagtacggcaaagaggttgaaaacggaggcaaaaccgctggagtaaaattaaaagaaggcgaggaagtcaggaaagaagccgtagaaagctcacttgaaagagctctatctttttactcatctatacaaacgagtgacggaaactgggcttccgatctaggtggaccaatgttcttactgcctggtttagttattgcactttatgtcacaggggttttaaactccgtattatctaaacatcatagacaagagatgtgtagatacgtttataatcatcaaaacgaggatggcggatgggggttacacatcgagggaccttcaacaatgtttggttcagctctaaactatgtagctcttaggttattgggcgaagatgctaatgctggtgcaatgcccaaagcaagagcatggatcttagatcatggaggtgccacggggattacaagttgggggaaattgtggctaagtgttctgggtgtatatgagtggtccggaaataatccactaccacccgaattctggttatttccatactttctgccttttcatccaggcagaatgtggtgtcattgcagaatggtctatttacctatgagttacctatacggtaaaagattcgtaggtccaattactcccatcgtgttgtctttgagaaaggaattatacgcagttccgtatcacgaaattgactggaataaatctagaaatacttgtgctaaagaagatctatactatcctcatcccaagatgcaagacattttgtggggaagtttacaccatgtctatgaacccttatttacaagatggcctgcgaaaagattgagagaaaaagcgctacagactgccatgcagcatattcattatgaagatgaaaacacaagatatatttgtttaggacctgtaaataaagtattgaatcttttatgttgttgggttgaagacccttattcagacgccttcaagttgcatttacaaagagtacatgactacttatgggtcgctgaagacggaatgaaaatgcaaggctataatggaagtcagctgtgggacacagccttttcaatacaagcaattgtttctaccaagctagtagataactacggcccaactttgagaaaggcccatgactttgttaagtccagccagatccaacaagattgtcctggtgatccaaacgtctggtataggcatattcacaagggtgcctggccctttagcactagagaccatggttggttgatttccgactgtacggccgaaggcttaaaagctgcattgatgctaagtaagttaccctccgaaacagtaggggagagtttagaaagaaatagactatgcgacgctgtaaatgtcttattatctttacaaaacgacaatggaggttttgcttcatacgaattaacaagatcctacccttggttagaactgattaacccagctgaaacttttggtgatattgtcatcgattatccctatgttgaatgtacgtctgcgactatggaagccttgactttatttaagaaacttcatccaggccacaggactaaggagatagatactgctattgttcgtgcggctaacttcttggaaaacatgcaaagaactgatggaagttggtacggttgttggggggtgtgtttcacatatgctggctggtttggaataaagggtttggttgccgctgggagaacgtataataattgtttagcaataaggaaagcttgcgactttcttttgagtaaggaattacctggcggtggatggggagagtcttacctttcatgccaaaataaggtgtacacgaacctagaaggtaatagacctcacttggtaaataccgcctgggttttaatggccttgatcgaagcaggacaagccgagagagatccaacaccattgcatcgtgctgccagactattaataaatagtcaactagagaacggtgacttcccacagcaagaaatcatgggtgtttttaataaaaactgtatgataacttatgccgcatatcgtaatatattcccaatttgggcgttaggagagtattgtcacagagtacttactgaa

SEQ ID NO: 12 - protein, 759 aa, Siraitia grosvenori
MWRLKVGAESVGENDEKWLKSISNHLGRQVWEFCPDAGTQQQLLQVHKARKAFHDDRFHRKQSSDLFITIQYGKEVENGGKTAGVKLKEGEEVRKEAVESSLERALSFYSSIQTSDGNWASDLGGPMFLLPGLVIALYVTGVLNSVLSKHHRQEMCRYVYNHQNEDGGWGLHIEGPSTMFGSALNYVALRLLGEDANAGAMPKARAWILDHGGATGITSWGKLWLSVLGVYEWSGNNPLPPEFWLFPYFLPFHPGRMWCHCRMVYLPMSYLYGKRFVGPITPIVLSLRKELYAVPYHEIDWNKSRNTCAKEDLYYPHPKMQDILWGSLHHVYEPLFTRWPAKRLREKALQTAMQHIHYEDENTRYICLGPVNKVLNLLCCWVEDPYSDAFKLHLQRVHDYLWVAEDGMKMQGYNGSQLWDTAFSIQAIVSTKLVDNYGPTLRKAHDFVKSSQIQQDCPGDPNVWYRHIHKGAWPFSTRDHGWLISDCTAEGLKAALMLSKLPSETVGESLERNRLCDAVNVLLSLQNDNGGFASYELTRSYPWLELINPAETFGDIVIDYPYVECTSATMEALTLFKKLHPGHRTKEIDTAIVRAANFLENMQRTDGSWYGCWGVCFTYAGWFGIKGLVAAGRTYNNCLAIRKACDFLLSKELPGGGWGESYLSCQNKVYTNLEGNRPHLVNTAWVLMALIEAGQAERDPTPLHRAARLLINSQLENGDFPQQEIMGVFNKNCMITYAAYRNIFPIWALGEYCHRVLTE

SEQ ID NO: 13 - DNA, 1575 bp, synthetic construct
  misc_feature 1..1575: note = Optimised Nucleic Acid sequence encoding squalene epoxidase
  source 1..1575: mol_type = other DNA
atggtcgatcagtgtgctctgggctggatcctggcgtcagtgcttggtgcagctgcattatatttcctatttggaagaaaaaacgggggagtttcaaacgaaagaagacatgaatcaattaagaacattgcaactacgaatggtgaatacaagtcttccaactctgatggtgatatcattatcgtgggcgccggtgtagcaggatctgctttggcatatacattagggaaagacggtcgtagagttcatgtgatagagagagatcttacggaaccggacaggattgttggtgaacttttgcagccgggcggatacttaaaactgacggaactaggtttagaggattgcgttgatgacattgatgctcaaagagtttacggctacgccttgttcaaagacggtaaagatactaggttaagttatcctctagaaaaatttcattccgacgttgctggcagatcctttcacaacggaagatttatacaaagaatgagagaaaaagccgctagtctaccaaatgtatccttggaacaaggtacagttacatccttattagaagagaatggtattatcaaaggcgtgcaatataaaacgaaaaccggacaagaaatgactgcttatgccccattaacaatcgtgtgtgatggctgcttctctaatttgagaagatctctttgtaatccaaaggttgacgttcctagctgctttgttggtttggttttggaaaattgcgatttaccgtatgctaaccatggacatgttatcctagcagatccgtctccaattctgttctacagaattagttcaactgaaattagatgtttggttgatgtccctggtcagaaggttccaagtatctccaacggtgaaatggctaattacctaaaaaacgttgttgctccgcaaattcccagccagttgtacgactctttcgttgccgcgatagacaaaggtaatatcagaacgatgccgaataggtccatgcctgctgacccatatcccaccccaggagcgttattgatgggtgatgcttttaatatgagacatccattaacaggcggagggatgactgttgctttgtctgatgttgtcgtcttgagagatttattaaaaccgcttcgtgatctaaacgatgcacctacattgtcaaagtatttagaggccttttacacgttgaggaagcctgttgctagtaccattaatacgttggctggagctctgtataaggtgttttgcgcctcaccagaccaagctagaaaagaaatgcgtcaagcttgtttcgactacctaagtctgggtggtatatttagtaacggtcctgtctctctattgtcagggctaaacccacgtcctattagtcttgtcttgcacttcttcgcagtggcgatttatggtgttggtaggttgctgattccgtttcccagtcctaaacgtgtgtggataggtgcaagaattatctctggtgcgtcagcgattatttttccaattattaaggctgaaggtgtgagacaaatgtttttccctgctactgttgccgcgtattacagagcaccaagggttgtcaagggcagataa

SEQ ID NO: 14 - protein, 524 aa, Siraitia grosvenori
MVDQCALGWILASVLGAAALYFLFGRKNGGVSNERRHESIKNIATTNGEYKSSNSDGDIIIVGAGVAGSALAYTLGKDGRRVHVIERDLTEPDRIVGELLQPGGYLKLTELGLEDCVDDIDAQRVYGYALFKDGKDTRLSYPLEKFHSDVAGRSFHNGRFIQRMREKAASLPNVSLEQGTVTSLLEENGIIKGVQYKTKTGQEMTAYAPLTIVCDGCFSNLRRSLCNPKVDVPSCFVGLVLENCDLPYANHGHVILADPSPILFYRISSTEIRCLVDVPGQKVPSISNGEMANYLKNVVAPQIPSQLYDSFVAAIDKGNIRTMPNRSMPADPYPTPGALLMGDAFNMRHPLTGGGMTVALSDVVVLRDLLKPLRDLNDAPTLSKYLEAFYTLRKPVASTINTLAGALYKVFCASPDQARKEMRQACFDYLSLGGIFSNGPVSLLSGLNPRPISLVLHFFAVAIYGVGRLLIPFPSPKRVWIGARIISGASAIIFPIIKAEGVRQMFFPATVAAYYRAPRVVKGR

SEQ ID NO: 15 - DNA, 1587 bp, synthetic construct
  misc_feature 1..1587: note = Optimised Nucleic Acid sequence encoding squalene epoxidase
  source 1..1587: mol_type = other DNA
atggttgatcaatgtgccttaggttggatcttagcttccgctttgggtttagtcattgctttgtgtttcttcgttgccccaagaagaaaccatagaggtgtcgattccaaagaaagagatgaatgtgtccaatctgctgctactaccaagggtgaatgcagatttaacgacagagatgtcgatgtcattgttgttggtgctggtgttgccggttctgctttggctcacactttgggtaaggatggtagaagagttcatgttatcgaaagagacttaaccgaaccagacagaattgttggtgagttgttgcaaccaggtggttacttgaaattgattgagttgggtttgcaagactgtgttgaagagatcgacgctcaaagagtttacggttatgctttattcaaggacggtaagaatacccgtttatcctacccattggaaaatttccattctgacgtttctggtagatctttccacaacggtagatttattcaaagaatgagagaaaaagccgcttctttaccaaacgttagattggaacaaggtactgttacttctttgttagaagaaaaaggtaccattaaaggtgttcaatacaagtccaaaaacggtgaagaaaagaccgcttacgctcctttgaccatcgtttgtgacggttgtttttctaacttgagaagatctttgtgtaaccctatggttgatgttccatcttactttgtcggtttggttttggaaaattgtgaattgccatttgccaaccatggtcacgttattttgggtgacccttccccaattttgttctaccaaatttcccgtactgaaatcagatgtttggtcgatgttccaggtcaaaaagttccttctatcgccaacggtgaaatggagaagtatttaaagaccgtcgttgctccacaagtccctccacaaatttacgactccttcatcgctgctattgacaagggtaacatcagaactatgccaaatagatccatgccagctgctccacacccaaccccaggtgccttattaatgggtgacgcttttaacatgcgtcacccattgaccggtggtggtatgactgtcgctttgtctgacattgttgtcttgcgtaacttattgaagccattgaaggacttgtctgacgcctctaccttgtgtaagtacttggaatccttctacactttgagaaagccagttgcttccactatcaacaccttggctggtgccttgtacaaggttttctgtgcttctccagaccaagctagaaaagaaatgagacaagcttgttttgactacttatctttgggtggtattttctctaacggtccagtttccttgttgtccggtttgaaccctagaccattgtccttagttttgcactttttcgccgtcgctatctacggtgttggtagattgttgttgccattcccttctgtcaagggtatctggattggtgctagattgatctactctgcttctggtattatcttcccaatcattagagccgaaggtgtcagacaaatgttcttcccagccactgttcctgcttactaccgttccccaccagttttcaagccaatcgtttaa

SEQ ID NO: 16 - protein, 528 aa, Siraitia grosvenori
MVDQCALGWILASALGLVIALCFFVAPRRNHRGVDSKERDECVQSAATTKGECRFNDRDVDVIVVGAGVAGSALAHTLGKDGRRVHVIERDLTEPDRIVGELLQPGGYLKLIELGLQDCVEEIDAQRVYGYALFKDGKNTRLSYPLENFHSDVSGRSFHNGRFIQRMREKAASLPNVRLEQGTVTSLLEEKGTIKGVQYKSKNGEEKTAYAPLTIVCDGCFSNLRRSLCNPMVDVPSYFVGLVLENCELPFANHGHVILGDPSPILFYQISRTEIRCLVDVPGQKVPSIANGEMEKYLKTVVAPQVPPQIYDSFIAAIDKGNIRTMPNRSMPAAPHPTPGALLMGDAFNMRHPLTGGGMTVALSDIVVLRNLLKPLKDLSDASTLCKYLESFYTLRKPVASTINTLAGALYKVFCASPDQARKEMRQACFDYLSLGGIFSNGPVSLLSGLNPRPLSLVLHFFAVAIYGVGRLLLPFPSVKGIWIGARLIYSASGIIFPIIRAEGVRQMFFPATVPAYYRSPPVFKPIV

SEQ ID NO: 17 - DNA, 951 bp, synthetic construct
  misc_feature 1..951: note = Optimised Nucleic Acid sequence encoding epoxy hydratase
  source 1..951: mol_type = other DNA
atggaaaacatcgaacacaccactgtccaaactaacggtatcaagatgcacgtcgctgctattggtactggtccaccagttttgttgttgcatggtttcccagaattgtggtattcttggagacaccaattgttgtacttgtcttctgctggttacagagctatcgctccagacttgagaggttacggtgataccgacgctccaccttccccatcttcctataccgctttacacattgtcggtgatttggtcggtttgttggacgtcttgggtatcgaaaaagtcttcttaatcggtcacgactggggtgccatcatcgcctggtacttctgtttattcagacctgatagaatcaaagctttggttaacttgtctgttcaattcttcccaagaaacccaaccaccccatttgttaagggtttcagagccgtcttgggtgatcaattttacatggttagattccaagaaccaggtaaagctgaagaagaattcgcttccgttgatattagagaattcttcaagaatgttttgtccaacagagatccacaagctccatatttgccaaatgaagttaagttcgaaggtgtcccaccaccagctttggctccatggttgaccccagaagatatcgatgtctacgctgacaaattcgctgaaactggtttcactggtggtttgaactactacagagcctttgacagaacctgggaattaactgctccatggaccggtgcccgtattggtgtcccagtcaagttcattgtcggtgatttggacttgacttaccactttccaggtgctcaaaaatacattcacggtgaaggtttcaagaaggctgtcccaggtttggaagaagttgtcgttatggaggatacctctcacttcattaaccaagaaagaccacacgaaattaattctcacatccacgatttcttctctaagttctgttaa

SEQ ID NO: 18 - protein, 316 aa, Siraitia grosvenori
MENIEHTTVQTNGIKMHVAAIGTGPPVLLLHGFPELWYSWRHQLLYLSSAGYRAIAPDLRGYGDTDAPPSPSSYTALHIVGDLVGLLDVLGIEKVFLIGHDWGAIIAWYFCLFRPDRIKALVNLSVQFFPRNPTTPFVKGFRAVLGDQFYMVRFQEPGKAEEEFASVDIREFFKNVLSNRDPQAPYLPNEVKFEGVPPPALAPWLTPEDIDVYADKFAETGFTGGLNYYRAFDRTWELTAPWTGARIGVPVKFIVGDLDLTYHFPGAQKYIHGEGFKKAVPGLEEVVVMEDTSHFINQERPHEINSHIHDFFSKFC

SEQ ID NO: 19 - DNA, 951 bp, synthetic construct
  misc_feature 1..951: note = Optimised Nucleic Acid sequence encoding epoxy hydratase
  source 1..951: mol_type = other DNA
atggatcaaattgaacacatcactattaacaccaacggtatcaaaatgcatatcgcctctgttggtactggtcctgttgttttgttgttgcatggtttcccagaattgtggtactcttggcgtcatcaattgttgtatttgtcctctgtcggttacagagctattgctccagatttaagaggttacggtgatactgactctccagcctctccaacttcttataccgctttgcatatcgttggtgacttggtcggtgctttggatgaattgggtattgaaaaggtcttcttggtcggtcatgattgggctgccatcatcgcttggtacttttgtttgtttagaccagatcgtattaaagctttagttaatttgtctgttcaattcatcccaagaaacccagctatcccatttattgaaggtttcagaaccgcttttggtgatgatttctacatgtgtagattccaagttccaggtgaagctgaagaagactttgcttctattgatactgctcaattgttcaaaacctccttgtgtaacagatcctccgctccaccatgcttgccaaaagaaatcggtttcagagctattccaccaccagaaaatttgccatcttggttgaccgaggaagacattaattactacgctgctaagttcaagcaaaccggtttcactggtgctttaaactactatagagctttcgatttgacctgggaattaactgctccatggaccggtgctcaaattcaagtcccagtcaagttcattgttggtgattctgacttgacttaccattttccaggtgctaaggaatacatccacaacggtggtttcaagaaggacgttccattgttggaagaagttgttgttgtcaaggacgcttgtcacttcatcaaccaagaaagaccacaagaaattaacgctcacattcatgactttattaacaagttctaa

SEQ ID NO: 20 - protein, 316 aa, Siraitia grosvenori
MDQIEHITINTNGIKMHIASVGTGPVVLLLHGFPELWYSWRHQLLYLSSVGYRAIAPDLRGYGDTDSPASPTSYTALHIVGDLVGALDELGIEKVFLVGHDWAAIIAWYFCLFRPDRIKALVNLSVQFIPRNPAIPFIEGFRTAFGDDFYMCRFQVPGEAEEDFASIDTAQLFKTSLCNRSSAPPCLPKEIGFRAIPPPENLPSWLTEEDINYYAAKFKQTGFTGALNYYRAFDLTWELTAPWTGAQIQVPVKFIVGDSDLTYHFPGAKEYIHNGGFKKDVPLLEEVVVVKDACHFINQERPQEINAHIHDFINKF

SEQ ID NO: 21 - DNA, 945 bp, synthetic construct
  misc_feature 1..945: note = Optimised Nucleic Acid sequence encoding epoxy hydratase
  source 1..945: mol_type = other DNA
atggagaagattgaacactctactatcgctactaatggtatcaatatgcacgttgcctctgctggttctggtccagctgttttgtttttgcacggtttcccagaattatggtattcctggagacaccaattgttgtacttgtcttctttgggttacagagctattgctccagatttgagaggtttcggtgacaccgatgctccaccatctccatcctcctacaccgcccaccacatcgttggtgatttggtcggtttgttggatcaattaggtgtcgatcaagtctttttggttggtcatgattggggtgctatgatggcctggtacttctgtttgttccgtccagacagagtcaaggccttagttaatttatctgtccacttcaccccacgtaacccagctatctctccattagatggtttccgtttgatgttgggtgatgatttctacgtttgtaagtttcaagaaccaggtgtcgctgaagccgatttcggttctgttgatactgccactatgtttaaaaagttcttgaccatgagagatccacgtccacctattattccaaacggtttcagatccttggccaccccagaagctttgccatcctggttgactgaagaggatatcgattactttgctgccaaattcgctaagactggttttactggtggtttcaactactacagagctatcgacttgacctgggagttgactgctccatggtccggttctgaaatcaaggttccaactaagtttattgttggtgacttagacttggtttaccatttcccaggtgttaaggaatacattcacggtggtggtttcaagaaggacgttccattcttggaagaagttgtcgtcatggaaggtgctgctcattttatcaaccaagaaaaagctgacgaaattaattctttgatctatgacttcattaaacaattctag

SEQ ID NO: 22 - protein, 314 aa, Siraitia grosvenori
MEKIEHSTIATNGINMHVASAGSGPAVLFLHGFPELWYSWRHQLLYLSSLGYRAIAPDLRGFGDTDAPPSPSSYTAHHIVGDLVGLLDQLGVDQVFLVGHDWGAMMAWYFCLFRPDRVKALVNLSVHFTPRNPAISPLDGFRLMLGDDFYVCKFQEPGVAEADFGSVDTATMFKKFLTMRDPRPPIIPNGFRSLATPEALPSWLTEEDIDYFAAKFAKTGFTGGFNYYRAIDLTWELTAPWSGSEIKVPTKFIVGDLDLVYHFPGVKEYIHGGGFKKDVPFLEEVVVMEGAAHFINQEKADEINSLIYDFIKQF

SEQ ID NO: 23 - DNA, 951 bp, synthetic construct
  misc_feature 1..951: note = Optimised Nucleic Acid sequence encoding epoxy hydratase
  source 1..951: mol_type = other DNA
atggaaaagattgaacacaccactatttctaccaatggtatcaacatgcatgttgcctctattggttctggtccagctgtcttgttcttgcacggtttcccagaattgtggtattcttggagacaccaattattattcttgtcttccatgggttacagagctatcgctccagacttaagaggttttggtgacaccgacgctccaccatctccatcttcttacaccgctcaccacattgtcggtgacttggtcggtttgttagaccaattgggtattgaccaagtttttttggttggtcacgactggggtgctatgatggcctggtacttttgtttgttccgtccagatagagttaaggctttggtcaatttatctgtccacttcttacgtagacacccatctatcaaatttgttgatggtttcagagccttattaggtgatgatttttacttctgtcaattccaagaaccaggtgtcgctgaagccgacttcggttctgtcgatgttgctaccatgttgaagaaattcttgaccatgagagatccaagacctccaatgattcctaaggaaaagggtttcagagccttggaaactccagatccattgccagcctggttaactgaagaagacattgactacttcgccggtaagtttcgtaagaccggttttaccggtggttttaattactacagagccttcaacttgacttgggagttgaccgctccatggtctggttctgaaatcaaggtcgctgccaagttcattgttggtgatttagacttggtttatcacttccctggtgccaaggagtatatccatggtggtggtttcaaaaaggacgtccctttgttggaggaagttgttgttgttgatggtgctgctcacttcatcaaccaagaaagaccagctgaaatttcttccttgatttacgactttatcaagaagttctaa

SEQ ID NO: 24 - protein, 316 aa, Siraitia grosvenori
MEKIEHTTISTNGINMHVASIGSGPAVLFLHGFPELWYSWRHQLLFLSSMGYRAIAPDLRGFGDTDAPPSPSSYTAHHIVGDLVGLLDQLGIDQVFLVGHDWGAMMAWYFCLFRPDRVKALVNLSVHFLRRHPSIKFVDGFRALLGDDFYFCQFQEPGVAEADFGSVDVATMLKKFLTMRDPRPPMIPKEKGFRALETPDPLPAWLTEEDIDYFAGKFRKTGFTGGFNYYRAFNLTWELTAPWSGSEIKVAAKFIVGDLDLVYHFPGAKEYIHGGGFKKDVPLLEEVVVVDGAAHFINQERPAEISSLIYDFIKKF

SEQ ID NO: 25 - DNA, 1599 bp, Siraitia grosvenori
  source 1..1599: mol_type = genomic DNA
atgctgatgcacgctctcacccctacagctcctttcttctctataaaacctaacacagaacccccttctgctaccacacggcagccacccatggattccccaccccaaaaacctcacttccttctcttccctttcatggctcagggccacatgatccccatgattgaccttgccaagcttctggcccagcgaggagccattattactgtcgtcaccacgccccacaatgctgctcgctaccactctgttctcgctcgcgccattgattctgggttacacatccatgtcctccaacttcagtttccatgcaacgaaggcgggttgccagaagggtgcgagaatttcgacttgttaccttcacttggttctgcctccacattcttcagagcaacattcctcctttacgaaccatcggaaaaagtgttcgaggaactcatcccccgccccacctgcataatctccgatatgtgtctgccctggaccgtacgacttgctcagaaatatcacgtcccaaggctcgttttctacagtttgagctgcttctttcttctctgtatgcggagtttaaaaaacaatcaagctcttataagctccaagtctgattctgagttggtaactttctcagacttgcctgatccagtcgagtttctcaagtcgcagctgcctaaatccaacgatgaagaaatggcaaagtttggttatgaaataggggaggccgatcggcaatcacacggcgttattgtgaatgtatttgaggagatggagccgaagtatcttgcggagtatagaaaggaaagagaatcgccggaaaaagtgtggtgcgtcggcccagtttcgctttgcaacgacaacaaactcgacaaggctcagagaggcaacaaagcctccatcgacgaacgcgaatgcatcgagtggctcgacgggcagcagccgtcttcagtggtttacgtgtctttaggaagtctgtgcaatttggtgacggcgcaacttattgagctgggtttgggtttggaggcatcaaacaaaccattcatttgggtcatacgaaaaggaaacataacagaggagttacagaaatggctggtggagtatgatttcgaggagaaaactaaagggagagggctcgtgattcttggctgggctccccaagttctgatactatcgcaccctgcaatcggatgctttttgacgcactgcggttggaactcaagcatcgaaggaatatcggccggcatgcccatgatcacttggccactttttgccgatcaagtcttcaacgagaagctaatcgtagagatactcagaatcggtgtaagtgtgggcatggaaacagctatgcactggggagaggaagaggagaaaggggtggtggtaaagagagagaaagtgagagaagccatagaaagggcgatggatggagatgagagagaagagaggagggagagatgcaaagagcttgctgaaatggcgaagagagccgtagaagaaggggggtcgtctcatcggaacctgacgctgctaactgaagatattcttgttaatggaggaggtcaagagagaatggatgatgctgatgattttcctactatagttaattga

SEQ ID NO: 26 - protein, 502 aa, Siraitia grosvenori
MDSPPQKPHFLLFPFMAQGHMIPMIDLAKLLAQRGAIITVVTTPHNAARYHSVLARAIDSGLHIHVLQLQFPCNEGGLPEGCENFDLLPSLGSASTFFRATFLLYEPSEKVFEELIPRPTCIISDMCLPWTVRLAQKYHVPRLVFYSLSCFFLLCMRSLKNNQALISSKSDSELVTFSDLPDPVEFLKSQLPKSNDEEMAKFGYEIGEADRQSHGVIVNVFEEMEPKYLAEYRKERESPEKVWCVGPVSLCNDNKLDKAQRGNKASIDERECIEWLDGQQPSSVVYVSLGSLCNLVTAQLIELGLGLEASNKPFIWVIRKGNITEELQKWLVEYDFEEKTKGRGLVILGWAPQVLILSHPAIGCFLTHCGWNSSIEGISAGMPMITWPLFADQVFNEKLIVEILRIGVSVGMETAMHWGEEEEKGVVVKREKVREAIERAMDGDEREERRERCKELAEMAKRAVEEGGSSHRNLTLLTEDILVNGGGQERMDDADDFPTIVN

SEQ ID NO: 27 - DNA, 1575 bp, Siraitia grosvenori
  source 1..1575: mol_type = genomic DNA
atggagaaactgcaggagaagcttcggaaaataaaacgtgctgtccacggggctggcactctccaaagaccaaaatgctattgtcaagtcaagctcttcccatctccccaagcaattaaaagagaaagacctcactttctgctcttccctttcatggctcagggccacatgatccccatgattgacctcgccaagcttctggctcagcgaggagccattgtcactatcctcaccacgccccacaatgctgctcgcacccactcagttcttgctcgcgccatcgattctgggttacaaatccgtgtccgcccacttcagtttccatgcaaagaagccggcctgccagaagggtgcgagaatctcgacttgttaccttcacttggttctgcctccacattcttcagagcaacatgtctcctttacgacccatcggaaaaactgttcgaggaactcagcccccggccgacttgcataatctccgatatgtgtctgccctggaccatacgacttgctcagaaatatcacgtaccaaggctcgttttctacagtttgagctgcttctttcttctctgtatgcggagtttaaaaaacaatccagcgcttattagctccaagtctgattctgagttcgtaactttctctgacttgcctgatccagtcgagtttctcaagtcggagctacctaaatccaccgatgaagacttggtgaagtttagttatgaaatgggggaggccgatcggaagtcatacggcgttattttaaatatatttgaggagatggaaccaaagtatcttgcggaatatggaaacgaaagagaatcgccggaaaaagtctggtgcgtcggcccagtttcgctttgcaacgacaacaaactcgacaaggctcagagaggcaacaaagcctccatcgacgaacgtgaatgcatcaagtggctcggcgggcagcagccgtcttcagtggtttacgcgtctttaggaagcttatgcaatctggttacggcgcaattcatagagttgggtttgggtttggaggcatcaaataaaccatttatttgggtcattagaaaaggaaacataacagaagagctacaaaaatggcttgtggagtatgatttcgaggagaaaactaaagggagagggctggtgattcttggctgggctccccaagttctgatactgtcccacccttcaatcggatgctttttgacgcactgtggttggaactcaagcatcgaagggatatcggccggcgtgccaatggtcacctggccgcttttttcggatcaagtcttcaacgagaagctaattgtacaaatactcagaatcggcgtaagtgtaggcgcggaaactgctatgaactggggagaggaagaggagaaaggggtagtagtgaagagagagaaagtgagggaagccatagaaaggatgatggatggagatgagagagaagagaggagagagagatgcaaagagcttgctgagacggcgaagagagctatagaagaagggggctcgtctcaccggaacctcacgctgttgattgaagatataggtacttcattgaggaggttgtga

SEQ ID NO: 28 - protein, 488 aa, Siraitia grosvenori
MDSPPHRPHFLLFPFMAQGHMIPMIDLAKLLAQRGAIVTILTTPHNAARTHSVLARAIDSGLQIRVRPLQFPCKEAGLPEGCENLDLLPSLGSASTFFRATCLLYDPSEKLFEELSPRPTCIISDMCLPWTIRLAQKYHVPRLVFYSLSCFFLLCMRSLKNNPALISSKSDSEFVTFSDLPDPVEFLKSELPKSTDEDLVKFSYEMGEADRKSYGVILNIFEEMEPKYLAEYGNERESPEKVWCVGPVSLCNDNKLDKAQRGNKASIDERECIKWLGGQQPSSVVYASLGSLCNLVTAQFIELGLGLEASNKPFIWVIRKGNITEELQKWLVEYDFEEKTKGRGLVILGWAPQVLILSHPSIGCFLTHCGWNSSIEGISAGVPMVTWPLFSDQVFNEKLIVQILRIGVSVGAETAMNWGEEEEKGVVVKREKVREAIERMMDGDEREERRERCKELAETAKRAIEEGGSSHRNLTLLIEDIGTSLRRL

SEQ ID NO: 29 - DNA, 1416 bp, Siraitia grosvenori
  source 1..1416: mol_type = genomic DNA
atgatgagaaaccaccatttccttctggtatgtttcccttctcaaggctatataaacccttcccttcaactcgccaggcgactgataagcctcggcgttaatgtcaccttcgccaccaccgtcctcgccggccgccgcatgaagaacaaaacccaccaaactgcaacaacaccaggcttgtctttcgctactttctccgatggcttcgatgacgaaaccctcaaacccaacggcgacttgacccactacttctcggagctcaggcgctgcggctctgaatctctaacccatctcattacttctgcagcaaacgaaggtcgtccgattaccttcgtaatctatagcctcctgctctcttgggcggctgatattgccagcacatatgacatcccgtcagcacttttttttgctcagcctgcgacggttttggctttgtacttctattacttccatggttatggtgataccatttgcagcaaactccaagacccatcttcgtacatagaattaccaggtttgccgttgctcactagtcaggacatgccctctttcttctccccttccggcccccatgctttcattctccctccaatgagagagcaggctgaattcctcggccgacaaagccaaccaaaagtactagtgaacaccttcgacgcgttagaggcagacgccttgagagccattgataagttgaagatgttggcgattggacccttgattccatctgctttactgggtggaaacgattcctctgatgcatcattttgtggtgatctttttcaagtctcgtcagaggattatatagaatggttgaactccaagcctgactcgtcggtcgtttacatatcagttggatccatctgcgtgctgtctgatgaacaagaggacgagcttgtgcatgctttattaaacagtggccacacgttcttgtgggtaaagagatcgaaagagaacaacgaaggagtaaaacaagaaacagacgaggagaagttgaagaagctggaagagcaagggaaaatggtgtcgtggtgccgtcaagttgaagtgttgaaacaccctgcgttgggttgttttctgacgcactgtgggtggaactcgactattgaaagcttggtttcagggctgccggtggttgcttttccgcagcagatagatcaagccaccaacgcgaagctcatagaggacgtgtggaagacgggagtgagggtgaaggccaatacagaaggaattgtggagagggaagaaatcaggaggtgcttggatttggtgatggggagcagagatgggcaaaaggaagagatagagagaaatgccaaaaagtggaaagaattggctagacaggccatcggtgaaggtgggtcatcagattcgaatcttaagacttttctatgggagattgatctagaaatttag

SEQ ID NO: 30 - protein, 471 aa, Siraitia grosvenori
MMRNHHFLLVCFPSQGYINPSLQLARRLISLGVNVTFATTVLAGRRMKNKTHQTATTPGLSFATFSDGFDDETLKPNGDLTHYFSELRRCGSESLTHLITSAANEGRPITFVIYSLLLSWAADIASTYDIPSALFFAQPATVLALYFYYFHGYGDTICSKLQDPSSYIELPGLPLLTSQDMPSFFSPSGPHAFILPPMREQAEFLGRQSQPKVLVNTFDALEADALRAIDKLKMLAIGPLIPSALLGGNDSSDASFCGDLFQVSSEDYIEWLNSKPDSSVVYISVGSICVLSDEQEDELVHALLNSGHTFLWVKRSKENNEGVKQETDEEKLKKLEEQGKMVSWCRQVEVLKHPALGCFLTHCGWNSTIESLVSGLPVVAFPQQIDQATNAKLIEDVWKTGVRVKANTEGIVEREEIRRCLDLVMGSRDGQKEEIERNAKKWKELARQAIGEGGSSDSNLKTFLWEIDLEI

SEQ ID NO: 31 - DNA, 1458 bp, Siraitia grosvenori
  source 1..1458: mol_type = genomic DNA
atggcggagcaagctcatgatcttcttcacgtcctcctttttccgtttccggcggagggccacatcaagcccttcctctgtctcgccgagctcctctgcaacgccggcttccatgtcaccttcctcaacaccgactacaaccaccgccgcctccacaacctccatctcctcgccgcccgctttccctcacttcatttcgagtccatttccgacggcctcccgcccgatcagcctcgagatatactggaccccaagttttttatatccatctgtcaagtcactaaaccccttttccgggagctcctcctttcctacaaacgcatttccagtgtccagaccggccgcccgccaataacttgcgttattacagatgtgatttttcgttttccgatcgacgtagctgaagaactggatattcctgtgtttagtttctgtactttcagtgcccgtttcatgtttctttacttctggattcccaagctcattgaagatggccagcttccatacccaaacggcaatatcaaccagaaactctacggtgttgctcctgaggcggaaggccttttaagatgtaaagatttgccgggacattgggctttcgcagacgaactaaaagatgatcaacttaactttgtggaccagacaacggcgtcatctcgatcctccggtctcattctcaacacattcgacgacctcgaagctccatttctggggcgtctctccaccatctttaagaaaatctacgccgttggacccatccactctctgttgaactcccaccactgtgggctttggaaagaagatcacagttgcctggcgtggctcgactcccgggccgcgaaatccgtcgtgttcgtcagcttcgggagcttggtgaagataacaagtaggcagctgatggagttttggcatggcttgctcaacagtggaaagtcgttcctcttcgtgttgagatctgacgtagttgagggcgatgatgaaaaacaagtcgtcaaagaaatttacgagacgaaggcagaggggaaatggttggttgtggggtgggctccgcaagagaaggtgttagcccatgaagctgttggtggatttctgacccattcgggctggaactccattttagagagcattgctgctggggttcctatgatctcctgccccaaaattggagaccagtccagtaactgtacgtggatcagtaaagtatggaaaattgggcttgaaatggaggatcggtacgaccgggtttcggtcgaaacaatggttagatctataatggaacaagaaggtgagaaaatgcagaagacaattgcagaattagcaaaacaagctaagtataaagttagtaaagatggaacatcatatcaaaatttagaatgtttaatccaagatattaaaaaactgaaccaaattgagggttttatcaacaaccccaattttagtgatttattaagggtttag

SEQ ID NO: 32 - protein, 485 aa, Siraitia grosvenori
MAEQAHDLLHVLLFPFPAEGHIKPFLCLAELLCNAGFHVTFLNTDYNHRRLHNLHLLAARFPSLHFESISDGLPPDQPRDILDPKFFISICQVTKPLFRELLLSYKRISSVQTGRPPITCVITDVIFRFPIDVAEELDIPVFSFCTFSARFMFLYFWIPKLIEDGQLPYPNGNINQKLYGVAPEAEGLLRCKDLPGHWAFADELKDDQLNFVDQTTASSRSSGLILNTFDDLEAPFLGRLSTIFKKIYAVGPIHSLLNSHHCGLWKEDHSCLAWLDSRAAKSVVFVSFGSLVKITSRQLMEFWHGLLNSGKSFLFVLRSDVVEGDDEKQVVKEIYETKAEGKWLVVGWAPQEKVLAHEAVGGFLTHSGWNSILESIAAGVPMISCPKIGDQSSNCTWISKVWKIGLEMEDRYDRVSVETMVRSIMEQEGEKMQKTIAELAKQAKYKVSKDGTSYQNLECLIQDIKKLNQIEGFINNPNFSDLLRV

SEQ ID NO: 33 - DNA, 1425 bp, Siraitia grosvenori
  source 1..1425: mol_type = genomic DNA
atggtgcaacctcgggtactgctgtttcctttcccggcactgggccacgtgaagcccttcttatcactggcggagctgctttccgacgccggcatagacgtcgtcttcctcagcaccgagtataaccaccgtcggatctccaacactgaagccctagcctcccgcttcccgacgcttcatttcgaaactataccggatggcctgccgcctaatgagtcgcgcgctcttgccgacggcccactgtatttctccatgcgtgagggaactaaaccgagattccggcaactgattcaatctcttaacgacggtcgttggcccatcacctgtattatcactgacatcatgttatcttctccgattgaagtagcggaagaatttgggattccagtaattgccttctgcccctgcagtgctcgctacttatcgattcacttttttataccgaagctcgttgaggaaggtcaaattccatacgcagatgacgatccgattggagagatccagggggtgcccttgttcgaaggtcttttgcgacggaatcatttgcctggttcttggtctgataaatctgcagatatatctttctcgcatggcttgattaatcagacccttgcagctggtcgagcctcggctcttatactcaacaccttcgacgagctcgaagctccatttctgacccatctctcttccattttcaacaaaatctacaccattggacccctccatgctctgtccaaatcaaggctcggcgactcctcctcctccgcttctgccctctccggattctggaaagaggatagagcctgcatgtcctggctcgactgtcagccgccgagatctgtggttttcgtcagtttcgggagtacgatgaagatgaaagccgatgaattgagagagttctggtatgggttggtgagcagcgggaaaccgttcctctgcgtgttgagatccgacgttgtttccggcggagaagcggcggaattgatcgaacagatggcggaggaggagggagctggagggaagctgggaatggtagtggagtgggcagcgcaagagaaggtcctgagccaccctgccgtcggtgggtttttgacgcactgcgggtggaactcaacggtggaaagcattgccgcgggagttccgatgatgtgctggccgattctcggcgaccaacccagcaacgccacttggatcgacagagtgtggaaaattggggttgaaaggaacaatcgtgaatgggacaggttgacggtggagaagatggtgagagcattgatggaaggccaaaagagagtggagattcagagatcaatggagaagctttcaaagttggcaaatgagaaggttgtcaggggtgggttgtcttttgataacttggaagttctcgttgaagacatcaaaaaattgaaaccatataaattttaa

SEQ ID NO: 34 - protein, 419 aa, Siraitia grosvenori
MVQPRVLLFPFPALGHVKPFLSLAELLSDAGIDVVFLSTEYNHRRISNTEALASRFPTLHFETIPDGLPPNESRALADGPLYFSMREGTKPRFRQLIQSLNDGRWPITCIITDIMLSSPIEVAEEFGIPVIAFCPCSARYLSIHFFIPKLVEEGQIPYADDDPIGEIQGVPLFEGLLRRNHLPGSWSDKSADISFSHGLINQTLAAGRASALILNTFDELEAPFLTHLSSIFNKIYTIGPLHALSKSRLGDSSSSASALSGFWKEDRACMSWLDCQPPRSVVFVSFGSTMKMKADELREFWYGLVSSGKPFLCVLRSDVVSGGEAAELIEQMAEEEGAGGKLGMVVEWAAQEKVLSHPAVGGFLTHCGWNSTVESIAAGVPMMCWPILGDQPSNATWIDRVWKIGVERNNREWDRLTVE

SEQ ID NO: 35 - DNA, 1422 bp, Siraitia grosvenori
  source 1..1422: mol_type = genomic DNA
atggatgcccagcaaggtcacaccaccaccattttgatgcttccatgggtcggctacggccatctcttgcctttcctcgagctggccaaaagcctctccaggaggaaattattccacatctacttctgttcaacgtctgttagcctcgacgccattaaaccaaagcttcctccttctatctcttctgatgattccatccaacttgtggaacttcgtctcccttcttctcctgagttacctcctcatcttcacacaaccaacggccttccctctcacctcatgcccgctctccaccaagccttcgtcatggctgcccaacactttcaggtcattttacaaacacttgccccgcatctcctcatttatgacattctccaaccttgggctcctcaagtggcttcatccctcaacattccagccatcaacttcagtactaccggagcttcaatgctttctcgaacgcttcaccctactcactacccaagttctaaattcccaatctcagagtttgttcttcacaatcactggagagccatgtacaccaccgccgatggggctcttacagaagaaggccacaaaattgaagaaacacttgcgaattgcttgcatacttcttgcggggtagttttggtcaatagtttcagagagcttgagacgaaatatatcgattatctctctgttctcttgaacaagaaagttgttccggtcggtcctttggtttacgaaccgaatcaagaaggggaagatgaaggttattcaagcatcaaaaattggcttgacaaaaaggaaccgtcctcaaccgtcttcgtttcatttggaaccgaatacttcccgtcaaaggaagaaatggaagagatagcgtatgggttagagctgagcgaggttaatttcatctgggtccttagatttcctcaaggagacagcaccagcaccattgaagacgccttgccgaaggggtttctggagagagcgggagagagggcgatggtggtgaagggttgggctcctcaggcgaagatactgaagcattggagcacaggggggcttgtgagtcactgtggatggaactcgatgatggagggcatgatgtttggcgtacccataatagcggttccgatgcatctggaccagccctttaacgccggactcgtggaagaagctggcgtcggcgtggaagccaagcgagattcggacggcaaaattcaaagagaagaagttgcaaagtcgatcaaagaagtggtgattgagaaaaccagggaagacgtgaggaagaaagcaagagaaatggacaccaaacacggacctacctattttagtcggtcgaaagttagcagttttggaaggctatataaaatcaaccgaccaactacactgacggttggtcgattttggtcgaaacagatcaagatgaagcgagagtaa

SEQ ID NO: 36 - protein, 459 aa, Siraitia grosvenori
MDAQQGHTTTILMLPWVGYGHLLPFLELAKSLSRRKLFHIYFCSTSVSLDAIKPKLPPSISSDDSIQLVELRLPSSPELPPHLHTTNGLPSHLMPALHQAFVMAAQHFQVILQTLAPHLLIYDILQPWAPQVASSLNIPAINFSTTGASMLSRTLHPTHYPSSKFPISEFVLHNHWRAMYTTADGALTEEGHKIEETLANCLHTSCGVVLVNSFRELETKYIDYLSVLLNKKVVPVGPLVYEPNQEGEDEGYSSIKNWLDKKEPSSTVFVSFGTEYFPSKEEMEEIAYGLELSEVNFIWVLRFPQGDSTSTIEDALPKGFLERAGERAMVVKGWAPQAKILKHWSTGGLVSHCGWNSMMEGMMFGVPIIAVPMHLDQPFNAGLVEEAGVGVEAKRDSDGKIQREEVAKSIKEVVIEKTREDVRKKAREMGEILRSKGDEKIDELVAEISLLRKKAPCSI

SEQ ID NO: 37 - DNA, 1359 bp, Siraitia grosvenori
  source 1..1359: mol_type = genomic DNA
atggatgctgcccaacaaggtgacaccacaaccattttgatgcttccatggctcggctatggccatctttcagcttttctcgagctggccaaaagcctctcaaggaggaacttccatatctacttctgttcaacctctgttaatcttgacgccattaaaccaaagcttccttcttctttctctgattccattcaatttgtggagctccatctcccttcttctcctgagttccctcctcatcttcacacaaccaacggccttccccctaccctcatgcccgctctccaccaagccttctccatggctgcccagcactttgagtccattttacaaacacttgccccgcaccttctcatttatgactctcttcaaccttgggctcctcgggtagcttcatccctcaaaattccggccatcaacttcaataccacgggagttttcgtcatttctcaagggyttcaccctattcactacccacattctaaattcccattctcagagttcgttcttcacaatcattggaaagccatgtactccactgccgatggagcttctaccgaaagaacccgcaaacgtggagaagcgtttctgtattgcttgcatgcttcttgtagtgtaattctaatcaatagtttcagagagctcgaggggaaatatatggattatctctctgttctcttgaacaagaaagttgttccggttggtcctttggtttacgaaccgaatcaagacggggaagatgaaggttattcaagcatcaaaaattggcttgacaaaaaggaaccgtcctccaccgtcttcgtgtcatttggaagcgaatacttcccgtcaaaggaagaaatggaagagatagcccatgggttagaggcgagcgaggttaatttcatctgggtcgttaggtttcctcaaggagacaacaccagcggcattgaagatgccttgccgaagggttttctggagagggcgggagagagagggatggtggtgaagggttgggctcctcaggcgaagatactgaagcattggagcacagggggattcgtgagccactgtggatggaactcggtgatggagagcatgatgtttggcgttcccataataggggttccgatgcatgtggaccagccctttaacgccggactcgtggaagaagctggcgtcggcgtggaggccaagcgagatccagacggcaaaattcaaagagacgaagttgcaaagttgatcaaagaagtggtggttgagaaaaccagagaagatgtgcggaagaaagcaagagaaatgagtgagattttgaggagcaagggagaggagaagtttgatgagatggtcgctgaaatttctctcttgcttaaaatatga

SEQ ID NO: 38 - protein, 452 aa, Siraitia grosvenori
MDAAQQGDTTTILMLPWLGYGHLSAFLELAKSLSRRNFHIYFCSTSVNLDAIKPKLPSSFSDSIQFVELHLPSSPEFPPHLHTTNGLPPTLMPALHQAFSMAAQHFESILQTLAPHLLIYDSLQPWAPRVASSLKIPAINFNTTGVFVISQGLHPIHYPHSKFPFSEFVLHNHWKAMYSTADGASTERTRKRGEAFLYCLHASCSVILINSFRELEGKYMDYLSVLLNKKVVPVGPLVYEPNQDGEDEGYSSIKNWLDKKEPSSTVFVSFGSEYFPSKEEMEEIAHGLEASEVNFIWVVRFPQGDNTSGIEDALPKGFLERAGERGMVVKGWAPQAKILKHWSTGGFVSHCGWNSVMESMMFGVPIIGVPMHVDQPFNAGLVEEAGVGVEAKRDPDGKIQRDEVAKLIKEVVVEKTREDVRKKAREMSEILRSKGEEKFDEMVAEISLLLKI

SEQ ID NO: 39 - protein, 317 aa, Siraitia grosvenori
MDAIEHRTVSVNGINMHVAEKGEGPVVLLLHGFPELWYSWRHQILALSSLGYRAVAPDLRGYGDTDAPGSISSYTCFHIVGDLVALVESLGMDRVFVVAHDWGAMIAWCLCLFRPEMVKAFVCLSVPFRQRNPKMKPVQSMRAFFGDDYYICRFQNPGEIEEEMAQVGAREVLRGILTSRRPGPPILPKGQAFRARPGASTALPSWLSEKDLSFFASKYDQKGFTGPLNYYRAMDLNWELTASWTGVQVKVPVKYIVGDVDMVFTTPGVKEYVNGGGFKKDVPFLQEVVIMEGVGHFINQEKPEEISSHIHDFISKF

SEQ ID NO: 40 - protein, 316 aa, Siraitia grosvenori
MDEIEHITINTNGIKMHIASVGTGPVVLLLHGFPELWYSWRHQLLYLSSVGYRAIAPDLRGYGDTDSPASPTSYTALHIVGDLVGALDELGIEKVFLVGHDWGAIIAWYFCLFRPDRIKALVNLSVQFIPRNPAIPFIEGFRTAFGDDFYICRFQVPGEAEEDFASIDTAQLFKTSLCNRSSAPPCLPKEIGFRAIPPPENLPSWLTEEDINFYAAKFKQTGFTGALNYYRAFDLTWELTAPWTGAQIQVPVKFIVGDSDLTYHFPGAKEYIHNGGFKRDVPLLEEVVVVKDACHFINQERPQEINAHIHDFINKF

SEQ ID NO: 41 - protein, 325 aa, Siraitia grosvenori
MEKESEIHSIRHTTVSVNGINMHVAEKGEGPLVLFIHGFPELWYSWRHQILDLASLGYRAVAPDLRGYGDSDAPPSASSYTSFHIVGDLIALLDAIVGVEEKVFVVAHDWGAIIAWYLCLYRPDRIKALVNLSVAFIRRNPKGKPVEWIRALYGDDHYMCRCQEPGEIEGEFAEIGTERVLTQFLTYHSPKPLMLPKGKAFGHPLDTPIPLPPWLSHQDIEYYASKFDKKGFTGPVNYYRNLDRNWELNAPFTRAQVKVPVKFIVGDLDLTYHSFGTKEYIHSGEMKKDVPFLQEVVVMEGVGHFIQSEKPHEISDHIYQFIKKF

SEQ ID NO: 42 - protein, 316 aa, Siraitia grosvenori
MEKIEHTIITTNGINMHVASIGTGPAVLFLHGFPELWYSWRHQLLSFSSLGYRAIAPDLRGYGDSDAPPSPSSYTVFHIVGDLVGLLDQLGIDQVFLVGHDWGASIAWYFSLLRPDRIKALVNLSVQYFPRNPARNTVEALRALFGDDYYVCRFQEPGEMEEDFASIDTAVIFKIFLSSRDPRPPCIPKAVGFRAFPVPDSLPSWLSEEDISYYASKFSKKGFTGGLNYYRALALNWELTAPWTGTQIKVPTKFIVGDLDLTYHIPGSKEYIHKGGFERDVPSLEEVVVIEGAAHFVNQERPEEISKHIYDFIKKF

SEQ ID NO: 43 - protein, 317 aa, Siraitia grosvenori
MDAIEHRTVSVNGINMHVAEKGEGPVVLLLHGFPELWYSWRHQILALSSLGYRAVAPDLRGYGDTDAPGSISSYTCFHIVGDLVALVESLGVDRVFVVAHDWGAMIAWCLCLFRPEMVKAFVCLSVPFRQRNPKMKPVQSMRAFFGDDYYICRFQNPGEIEEEMAQVGAREVLRGILTSRRPGPPILPKGQAFRARPGASTALPSWLSEKDLSFFASKYDQKGFTGPLNYYRAMDLNWELTASWTGVQVKVPVKYIVGDVDMVFTTPGVKEYVNGGGFKKDVPFLQEVVIMEGVGHFINQEKPEEISSHIHDFISRF

SEQ ID NO: 44 - protein, 311 aa, Siraitia grosvenori
MDQIQHKFIDIRGLKLHIAEIGTGSPAVVFLHGFPEIWYSWRHQMVAAAAVGYRAISPDLRGYGFSDPHPQPQNASFDDFVEDTLAILDFLHIPKAFLVGKDFGSWPVYLFSLVHPTRVAGIVSLGVPFLPPNPKRYRDLPEGFYIFRWKESGRAEADFGRFDVKTVLRRIYTLFSRSEIPIAEKDQEIMDMVDESTPPPPWLTDEDLAAYATAYEHSGFESALQVPYRRRHQELGMSNPRVDVPVLLIIGGKDYFLKFPGIEDYIKSEKMREIVPDLEVADLADGTHFMQEQFPAQVNHLLISFLGKRNT

SEQ ID NO: 45 - protein, 759 aa, Siraitia grosvenori; identical to SEQ ID NO: 12

SEQ ID NO: 46 - protein, 524 aa, Cucumis melo
MVDQCALGWILASVLGASALYLLFGKKNCGVLNERRRESLKNIATTNGECKSSNSDGDIIIVGAGVAGSALAYTLAKDGRQVHVIERDLSEPDRIVGELLQPGGYLKLTELGLEDCVDDIDAQRVYGYALFKDGKDTRLSYPLEKFHSDVSGRSFHNGRFIQRMREKAASLPNVRLEQGTVTSLLEENGTIKGVQYKNKSGQEMTAYAPLTIVCDGCFSNLRRSLCNPKVDVPSCFVGLILENCDLPYANHGHVILADPSPILFYPISSTEIRCLVDVPGQKVPSISNGEMANYLKNVVAPQIPPQLYNSFIAAIDKGNIRTMPNRSMPADPYPTPGALLMGDAFNMRHPLTGGGMTVALSDIVVLRDLLKPLRDLNDAPTLCKYLEAFYTLRKPVASTINTLAGALYKVFCASPDQARKEMRQACFDYLSLGGIFSNGPVSLLSGLNPRPLSLVLHFFAVAIYGVGRLLIPFPSPKRVWIGARLISGASAIIFPIIKAEGVRQMFFPKTVAAYYRAPPVVRER

SEQ ID NO: 47 - protein, 528 aa, Cucumis sativus
MVDHCTFGWIFSAFLAFVIAFSFFLSPRKNRRGRGTNSTPRRDCLSSSATTNGECRSVDGDADVIIVGAGVAGSALAHTLGKDGRRVHVIERDLTEPDRIVGELLQPGGYLKLIELGLQDCVEEIDAQKVYGYALFKDGKSTRLSYPLENFQSDVSGRSFHNGRFIQRMREKAAFLPNVRLEQGTVTSLLEEKGTITGVQYKSKNGEQKTAYAPLTIVCDGCFSNLRRSLCNPMVDVPSCFVGLVLENCQLPYANLGHVVLGDPSPILFYPISSTEIRCLVDVPGQKVPSISNGEMEKYLKTVVAPQVPPQIHDAFIAAIEKGNIRTMPNRSMPAAPQPTPGALLMGDAFNMRHPLTGGGMTVALSDIVVLRNLLKPLKDLNDAPTLCKYLESFYTLRKPVASTINTLAGALYKVFCASSDQARKEMRQACFDYLSLGGIFSNGPVSLLSGLNPRPLSLVLHFFAVAIYGVGRLLLPFPSPKGIWIGARLVYSASGIIFPIIKAEGVRQMFFPATVPAYYRTPPVFNS

SEQ ID NO: 48 - protein, 318 aa, Cucumis sativus
METINHITVQTNGINLHVATAGPVTGPPVLLLHGFPELWYSWRHQIIFLSSVGYRVIAPDLRGYGDSDAPPSSDTYTALHIVGDVVGLLNELGIDKVLLVGHDWGALIAWYFCLFRPDRIKASVILSVQFFPRNPKVSFVEGFKAVLGDQFYMVRFQEPGKAEKEFASVDIREFFKNVMSNRDPSAPYLPGEEKFEGVPPPSLAPWLTPQDIDYYAQKFSHSGFTGGLNYYRAFDRTWELTAPWTAAEIKVPVKFIVGDLDLTYHFPGGQDYINGDAFRKDVPGLEEVIVMKDTSHFINQERPDEINCHIHDFFNKFC

SEQ ID NO: 49 - protein, 316 aa, Cucumis melo
MDAIQHTTIKTNGIKMHIASVGNGPVVLLLHGFPELWYSWRHQLLYLSSVGYRAIAPDLRGYGDTDSPESHTSYTALHIVGDLVGALDELGIEKVFLVGHDWGAIIAWYFCLFRPERIKALVNLSVQFFPRNPAISFIQRFRAAYGDDFYMCRFQVPGEAEADFACIDTAQLFKTTLSNRSTKAPCLPKEYGFRAIPPPENLPSWLTEEDINYYAAKFKETGFTGALNYYRAFDLTWELTAPWTGVQIQVPVKFIVGDSDLTYHFKGAKEYIHEGGFKRDVPLLEEVVIVENAGHFVHEEKPHEINTHIHDFIKKF

SEQ ID NO: 50 - protein, 315 aa, Cucumis melo
MDKIQHSTISTNGINIHFASIGSGPVVLFLHGFPELWYSWRHQLLFLASKGFRAIAPDLRGFGDSDAPPSPSSYTPHHIVGDLIGLLDHLGIDQVFLVGHDWGAMMAWYFCLFRPDRVKALVNLSVHYTPRNPAGSPLAVTRRYLGDDFYICKFQEPGVAEADFGSVDTATMMKKFLTMRDPRPAIIPNGFKTLLETPEILPSWLTEEDIEYFASKFSKTGFTGGFNYYRALDITWELTGPWSRAQIKVPTKFIVGDLDLVYNFPGAKEYIHGGGFKKDVPLLEDVVVIEGAAHFINQEKPDEISSLIYDFITKF

SEQ ID NO: 51 - protein, 322 aa, Cucumis sativus
MEKIEHTTIPTNGINMHVASIGSGPAVLFLHGFPQLWYSWRHQLLFLASKGFRALAPDLRGFGDTDAPPSPSSYTFHHIIGDLIGLLDHFGLDKVFLVGHDWGAVIAWYFCLFRPDRVKALVNLSVHYLKRHPSINFVDGFRASAGENFYICQFQEAGVAEADFGSVDTATMMKKFMGMRDPVAPPIYNTKEKGFSSLETPNPLPCWLTEEDVDFFASKFSKTGFTGGFNYYRALNLSWELTAAWNGSKIEVPVKFIVGDLDLVYHFPGAKEYINGGEFKKDVPFLEEVVVIKDAAHFINQEKPHQINSLIYHFINKFVSSI

SEQ ID NO: 52 - protein, 473 aa, Siraitia grosvenori
MWTVVLGLATLFVAYYIHWINKWRDSKFNGVLPPGTMGLPLIGETIQLSRPSDSLDVHPFIQKKVERYGPIFKTCLAGRPVVVSADAEFNNYIMLQEGRAVEMWYLDTLSKFFGLDTEWLKALGLIHKYIRSITLNHFGAEALRERFLPFIEASSMEALHSWSTQPSVEVKNASALMVFRTSVNKMFGEDAKKLSGNIPGKFTKLLGGFLSLPLNFPGTTYHKCLKDMKEIQKKLREVVDDRLANVGPDVEDFLGQALKDKESEKFISEEFIIQLLFSISFASFESISTTLTLILKLLDEHPEVVKELEAEHEAIRKARADPDGPITWEEYKSMTFTLQVINETLRLGSVTPALLRKTVKDLQVKGYIIPEGWTIMLVTASRHRDPKVYKDPHIFNPWRWKDLDSITIQKNFMPFGGGLRHCAGAEYSKVYLCTFLHILCTKYRWTKLGGGTIARAHILSFEDGLHVKFTPKE

SEQ ID NO: 53 - protein, 480 aa, Cucumis melo
MASTHILLFPFMAQGHMIPMIDLAKLLAHHGFIITIVTTPHNADRYHSVLARATHSGLQIHVALLPFPSTQVGLPEGCENLDLLPLHLSSSMSAFCRATSLLYEPSEKLLQQLCPRPSCIISDMCLPWTLRLAQNHQIPRLVFYSLSCFFLLCMRSLKTNHSLVTSISDSEFLTLSDLPDPVEIRKSQLSRVKNEEMGKLSYEMVEADRLSHGVILNVFEEMEAEYVAEYRKNRDLPQKVWCVGPLSLCNDNKLDKAERGEKSSIHEDECIKWLNGQQPSSVVYVSMGSLCNLSTPQLVELGLGLEASKKPFIWVIRKGNLTEELQRWIMEYDFERKTEGWGLVIRGWAPQVAILSHSAIGGFLTHCGWNSSIEGIAAGVPMMTWPLFADQVFNAKLIVEVLKVGVSVGEETALHWGEEAEKEVMVKREEVREAIERVMDGENREEMKQRSKKLAEMAKRAVEEGGSSHRNLKRLIEEIV

SEQ ID NO: 54 - protein, 497 aa, Cucumis melo
MASTLSNQLELQPHFVLVPLMAQGHMIPMIDIATLLARRGVFVTFVTTPYNATRLESFFARAKQSSLSISLLEIPFPCLQVGLPLGCENLDTLPSRSLLRNFYKALSLLQQPLEQFLSRHHLNPTCIISDKYLYWTAQTAHKFKCPRVVFHGTGCFSLLSSHNLQLYSPHTSIDSNSQPFLVPGLPHKIEITKSQLPGSLIKSPDFDDFRDKITKAEQEAYGVVVNSFSELENGYYQNYERAISKKLWCIGPVSLCNENSIEKYNRGNKASIEQSNCLNWLDSMIPKSVLYICLGSLCRMLPSQLIQLGQCLESSTRPFIWVIKNRDENCSELEKWLSEEEFERKTKGRGLIIRGWAPQLLILSHWSTGGFLTHCGWNSTVEGIGNGVPMITWPQFAEQFLNEKLVVEILKIGVRVGVEGAVRWGEEERVGVMAKKEEIEKAIEMVMDGGEEGEERRRRVGDLSKMAPKAMENGGSSYVNLSLFIEDVMAQSAHLKA

SEQ ID NO: 55 - protein, 477 aa, Cucumis sativus
MDPKNTQLRIFFFPFMAQGHTIPAIDMAKLFASRGADVAIITTPLNAPLIAKSINKFDRPGRKIELLIIDFPSVAVGLPDGCESLDLARSPEMFQSFFRATTLLEPQIDQILDHHRPHCLVADTFFPWTTDLAAKYGIPRVVFHGTCFFALCAAASLIANRPYKKVSSDLEPFVIPGLPDEIKLTRSQVPGFLKEEVETDFIKLYWASKEVESRCYGFLINSFYELEPAYADYYRNVLGRRAWHIGPLSLYSNVEEDNVQRGSSSSISEDQCLKWLDSKNPDSVLYVSFGSLASLTNSQLLEIAKGLEGTGQNFIWVVKKAKGDQEEWLPEGFEKRVEGKGLIIRGWAPQVLILDHRSIGGFVTHCGWNSALEGVTAGVPMVTWPNSAEQFYNEKLITDVLQIGVGVGALYWGRAGKDEIKSEAIEKAVNRVMVGEEAEEMRSRAKALGIQARKAIVEGGSSSSDLNAFFKDLRSQI

SEQ ID NO: 56 - protein, 467 aa, Cucumis melo
MEMTAANGGGERIKQSHVIVFPFPRHGHMSPMLQFSKRLISKGLLLTFLITSSASQSLTINIPPSPSFHFKIISDLPESDDVATLDAYLRSFRAAVTKSLSNFIDEVLTSSSNEEVPPTLIVYDSVMPWVQSVAAERGLDSAPFFTESAAVNHLLHLVYGGSLSIPPPDNVVVSLPSEIVLQPEDLPSFPDDPEVVLDFMTSQFSHLENVKWIFINTFDRLESKVVNWMAKTLPIKTVGPTIPSAYLDGRLEKDKAYGLNVSKSNNGKCPIKWLDSKETASVIYISFGSLVILSEEQVKELTNLLRDTDFSFLWVLRESEMVKLPKNFVQDTSDRGLIVNWCCQLQVLSHKAVSCFVTHCGWNSTLEALSLGVPMVAIPQWIDQTTNAKFVADVWRVGVRVKKNEKSVAIKEELEASIRKIVVQGNGTNEFKQNAIKWKNLAKEAVDERGSSDKNIEEFVQALVASN

SEQ ID NO: 57 - protein, 464 aa, Cucumis sativus
MRNHHFLIVCFPSQGYINPSLQLANKLTSLNIEVTFATTVTASRRMKITQQISSPSTLSFATFSDGFDDENHKTSDFNHFFSELKRCGSQSLTDLITSFRDRHRRPFTFVIYSLLLNWAADVATSFNIPSALFSAQPATVLALYYYYFHGFEDEITNKLQNDGPSSLSIELPGLPLLFKSHEMPSFFSPSGQHAFIIPWMREQMEFLGQQKQPIKVLVNTFHALENEALRAIHELEMIAIGPLISQFRGDLFQVSNEDYYMEWLNSKSNCSVVYLSFGSICVLSKEQEEEILYGLFESGYPFLWVMRSKSDEDEEKWKELVEGKGKIVSWCRQIEVLKHPSLGCFMSHCGWNSTLESLSFGLPMVAFPQQVDQPTNAKLVEDVWKMGVRVKGNLEGIVEREEIRRCLDLVMNRKYINGEREETEKNVEKWKKLAWEAMDEGGSSILNLANFVDEIDVGDELADS

SEQ ID NO: 58 - protein, 471 aa, Cucumis sativus
MGLSPTDHVLLFPFPAKGHIKPFFCLAHLLCNAGLRVTFLSTEHHHQKLHNLTHLAAQIPSLHFQSISDGLSLDHPRNLLDGQLFKSMPQVTKPLFRQLLLSYKDGTSPITCVITDLILRFPMDVAQELDIPVFCFSTFSARFLFLYFSIPKLLEDGQIPYPEGNSNQVLHGIPGAEGLLRCKDLPGYWSVEAVANYNPMNFVNQTIATSKSHGLILNTFDELEVPFITNLSKIYKKVYTIGPIHSLLKKSVQTQYEFWKEDHSCLAWLDSQPPRSVMFVSFGSIVKLKSSQLKEFWNGLVDSGKAFLLVLRSDALVEETGEEDEKQKELVIKEIMETKEEGRWVIVNWAPQEKVLEHKAIGGFLTHSGWNSTLESVAVGVPMVSWPQIGDQPSNATWLSKVWKIGVEMEDSYDRSTVESKVRSIMEHEDKKMENAIVELAKRVDDRVSKEGTSYQNLQRLIEDIEGFKLN

SEQ ID NO: 59 - protein, 452 aa, Cucumis sativus
MDVQKSRDTPTTILMLPWIGYGHLSAYLELAKVLSRRNNFLIYFCSTPVNLDSIKPRLIPSSSIQFVELHLPSSPEFPPHLHTTNALPPRLTPTLHKAFAAAASPFEAILQTLCPHLLIYDSLQQWAPQIASSLNIPAINFNTTAASIISHALHNINYPDTKFPLSDWVLHNYWKGKYTTANEATLERIRRVRESFLYCLSASRDITLISSCREIEGEYMDYLSVLLKKKVIAVGPLVYEPREDDEDEDYSRIKNWLDKKEALSTVLVSFGSEFFPSKEEMEEIGCGLEESGANFIWVIRSPKGEENKRVEEALPEGFVEKAGERAMIVKEWAPQGKILKHRSIGGFVSHCGWNSVMESIMLGVPVIAVPMHVDQPYNAGLVEEAGLGVEAKRDPDGMIQREEVAKLIREVVVDKSREDLRTKVIEMGEILRSKGDEKIDEMVAQISLLLKI